(19) United States
(12) Patent Application Publication
(10) Pub. No.: US 2023/0338529 A1
(43) Pub. Date: Oct. 26, 2023

(54) COMPOSITIONS FOR ALTERING A MICROGLIAL CELL, AND METHODS OF USE THEREFORE

(71) Applicant: The Children's Medical Center Corporation, Boston, MA (US)

(72) Inventors: Zhigang HE, Boston, MA (US); Yi LI, Boston, MA (US); Xuelian HE, Boston, MA (US)

(73) Assignee: The Children's Medical Center Corporation, Boston, MA (US)

(21) Appl. No.: 18/019,627

(22) PCT Filed: Aug. 5, 2021
(86) PCT No.: PCT/US2021/044680

§ 371 Date: Feb. 3, 2023

Related U.S. Application Data
(60) Provisional application No. 63/062,088, filed on Aug. 6, 2020.

(51) Int. Cl. A61K 31/336; A61K 39/00; A61P 17/02; A61K 38/55; A61K 31/404; A61P 25/00

(52) U.S. Cl. CPC A61K 39/4614; A61K 31/336; A61K 31/404; A61K 38/55; A61K 39/4633; A61K 39/4637; A61K 39/464; A61K 39/4644; A61K 39/464402; A61K 39/464429; A61P 17/02; A61P 25/00

(57) ABSTRACT

Provided herein are compositions and methods for reducing neuroinflammation and treating neurodegenerative diseases using proteinase inhibitors. The invention also provides methods for reducing post-injury scar formation in the central nervous system.

### CROSS-REFERENCE TO RELATED APPLICATION

This application claims the benefit of the following U.S. Provisional Application No.: 63/062,088, filed Aug. 6, 2020, the entire contents of which is incorporated herein by reference.

### STATEMENT OF RIGHTS TO INVENTIONS MADE UNDER FEDERALLY

### SPONSORED RESEARCH

This invention was made with Government support under Grant Nos. NS096294 and NS110850 awarded by the National Institutes of Health. The Government has certain rights in the invention.

### BACKGROUND OF THE INVENTION

Neuroinflammation accompanies neuronal injury and neurodegenerative disease in the mammalian central nervous system (CNS). At early stages, neuroinflammation is protective, but at later stages it accelerates degeneration and hinders regeneration. The cells that orchestrate both helpful and harmful neuroinflammatory responses are microglia, so they likely hold the key to the switch between states.

Inflammatory processes in general, and diseases and disorders related to neuroinflammation, such as from spinal cord injury, are numerous, and the mechanisms and actions are still not well understood. Currently, there is an unmet need for an effective way of treating neuroinflammatory related diseases and disorders.

### SUMMARY OF THE INVENTION

As described below, the present invention features compositions and methods for reducing neuroinflammation and treating neurodegenerative diseases using proteinase inhibitors and/or phospholipase A2 inhibitor and/or a microglial cell or microglial-like cell contacted with a proteinase inhibitor and/or phospholipase A2 inhibitor. The invention also provides methods for reducing post-injury scar formation in the central nervous system.

In one aspect, the invention provides a method for reducing post-injury scar formation in the central nervous system of a subject. In some embodiments, the method includes contacting the site of injury with a proteinase inhibitor and/or phospholipase A2 inhibitor and/or a microglial cell or microglial-like cell contacted with a proteinase inhibitor and/or phospholipase A2 inhibitor, thereby reducing post-injury scar formation.

In one aspect, the invention provides a method for reducing neuroinflammation in a subject. In some embodiments, the method includes administering to the subject an effective amount of a proteinase inhibitor and/or phospholipase A2 inhibitor and/or a microglial cell or microglial-like cell contacted with a proteinase inhibitor and/or phospholipase A2 inhibitor, thereby reducing neuroinflammation in the subject.

In one aspect, the invention provides a method for treating neurodegeneration in a subject. In some embodiments, the method includes administering to the subject an effective amount of a proteinase inhibitor and/or phospholipase A2 inhibitor and/or an effective amount of a composition including a microglial cell or microglial-like cell contacted with a proteinase inhibitor and/or phospholipase A2 inhibitor, thereby treating the neurodegeneration.

In some embodiments, the proteinase inhibitor is a cysteine peptidase inhibitor. In some embodiments, the proteinase inhibitor is a serine protease inhibitor. In some embodiments, the cysteine peptidase inhibitor is E64. In some embodiments, the serine protease inhibitor is serpina3n. In some embodiments, the phospholipase A2 inhibitor is Varespladib.

In some embodiments, the microglial cell or microglial-like cell expresses a SPP1 and/or a CD68 polypeptide or a polynucleotide encoding said polypeptide. In some embodiments, the microglial cell or microglial-like cell fails to express or expresses reduced levels of a P2Y12 polypeptide or a polynucleotide encoding said polypeptide. In some embodiments, the microglial cell or microglial-like cell has an ameboid morphology. In some embodiments, the microglial cell or microglial-like cell expresses a polypeptide selected from the group consisting of Igf1, Ms4a7, Fabp5 Mif Ms4a7, Thbs1, Clec7a, Ms4a7, Ms4a6c, Lgals1, fibronectin 1 (Fn1), thrombospondin 1 (Thbs1), a phospholipase A2 inhibitor, Cstb, Stfa1 and Serpinb6a, 6Anxa1, or a polynucleotide encoding said polypeptide. In some embodiments, the microglial cell or microglial-like cell is derived from an induced pluripotent stem cell or embryonic stem cell. In some embodiments, the microglial cell or microglial-like cell is autologous or heterologous. In some embodiments, the heterologous microglial cell or microglial-like cell is derived from a mammal of the same or a different species than the subject. In some embodiments, the mammal is a human, bovine, porcine, canine, or feline.

In some embodiments, the proteinase inhibitor and/or phospholipase A2 inhibitor is administered locally or systemically. In some embodiments, the microglial cell or microglial-like cell is contacted with the proteinase inhibitor and/or phospholipase A2 inhibitor in vitro or in vivo. In some embodiments, the site of injury, neuroinflammation, or neurodegeneration is the brain, optic nerve, or spinal cord. In some embodiments, the injury is a traumatic injury or a post-surgical injury. In some embodiments, the method promotes axon regeneration or regrowth. In some embodiments, the proteinase inhibitor and/or phospholipase A2 inhibitor and the microglial cell or microglial-like cell treated with a proteinase inhibitor and/or phospholipase A2 inhibitor are administered concurrently or sequentially. In some embodiments, the proteinase inhibitor and/or phospholipase A2 inhibitor and/or the microglial cell or microglial-like cell treated with a proteinase inhibitor and/or phospholipase A2 inhibitor are administered prior to or subsequent to the injury, neuroinflammation, or neurodegeneration. In some embodiments, the proteinase inhibitor and/or phospholipase A2 inhibitor and/or the microglial cell or microglial-like cell treated with a proteinase inhibitor and/or phospholipase A2 inhibitor are administered within hours or days of the injury, neuroinflammation, or neurodegeneration. In some embodiments, the administration is within 1, 6, 12, or 24 hours of the injury, neuroinflammation, or neurodegeneration. In some embodiments, the administration is within 1, 3, 5, or 7 days of the injury, neuroinflammation, or neurodegeneration.

In some embodiments, the microglial cell or microglial-like cell is an activated microglial cell or microglial-like cell. In some embodiments, the microglial cell or microglial-like cell expresses one or more markers associated with an MG2 or MG3 microglial cell. In some embodiments, the step of administrating reduces the number of CD68+ cells, fibroblasts, reactive astrocytes, collagen I, fibronectin, CSPG and/or laminin present at the site of injury, neuroinflammation, or neurodegeneration relative to an untreated site of injury, neuroinflammation, or neurodegeneration. In some embodiments, the number of CD68+ cells, fibroblasts, or reactive astrocytes is reduced by at least about 10, 25, or 50% relative to an untreated site of injury, neuroinflammation, or neurodegeneration after administration. In some embodiments, the microglial cell or microglial-like cell is characterized as having the following polypeptide expression profile: CD68-, SPP1-, P2Y12+, TMEM119+. In some embodiments, the microglial cell or microglial-like cell is characterized as having the following polypeptide expression profile: CD68-, SPP1-, P2Y12+, and/or TMEM119+. In some embodiments, the microglial cell or microglial-like cell expresses fibronectin and associated binding proteins; and/or one or more extracellular or intracellular peptidase inhibitors and/or phospholipase A2 inhibitors.

In some embodiments, the neurodegeneration is associated with a disease selected from the group consisting of Alzheimer’s disease, Parkinson’s disease, glaucoma, metachromatic leuokodystrophy, adrenoleukodystophy, lysosomal storage disorders, multiple sclerosis and amyotrophic lateral sclerosis. In some embodiments, the neuroinflammation is associated with a neuroinflammatory disease. In some embodiments, the neuroinflammation is associated with neuronal injury. In some embodiments, the neuronal injury is selected from the group consisting of traumatic brain injury, spinal cord injury, spinal cord crush, and optic nerve injury. In some embodiments, the subject is a human subject.

In another aspect, the invention provides a pharmaceutical composition including an amount of a peptide inhibitor and/or phospholipase A2 inhibitor in an amount effective to reduce post-injury scar formation in the central nervous system of a subject, reduce inflammation, or treat neurodegeneration.

In yet another aspect, the invention provides a pharmaceutical composition including a microglial cell or microglial like cell treated with one or more proteinase inhibitors and/or one or more phospholipase A2 inhibitors. In some embodiments, the microglial cell or microglial like cell is characterized as having the following polypeptide expression profile: CD68-, SPP1-, P2Y12+ TMEM119+. In some embodiments, the microglial cell or microglial like cell is characterized as having the following polypeptide expression profile: CD68-, SPP1-, P2Y12+, and/or TMEM119+. In some embodiments, any of the pharmaceutical compositions as provided herein further includes a pharmaceutically acceptable carrier, diluent, excipient, or vehicle.

In one aspect, the invention provides an isolated microglial-like cell treated with a proteinase inhibitor and/or phospholipase A2 inhibitor and characterized as having one of the following polypeptide expression profiles: (i) CD68-, SPP1-, P2Y12+, TMEM119+; or (ii) CD68-, SPP1-, P2Y12+, and/or TMEM119+. In yet another aspect, the invention provides a kit including any of the pharmaceutical compositions as provided herein. In yet another aspect, the invention provides a kit including any of the treated microglial or microglial-like cells as provided herein. In some embodiments, the kit further includes instructions for using any of the pharmaceutical compositions or treated microglial or microglial-like cells as provided herein.

The description and examples herein illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope.

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M.J. MacPherson, B.D. Hames and G.R. Taylor eds. (1995)), Harlow and Lane, eds. (1988) Antibodies, A Laboratory Manual, and Culture of Animal Cells: A Manual of Basic Technique and Specialized Applications, 6th Edition (R.I. Freshney, ed. (2010)).

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment. The section headings used herein are for organizational purposes only and are not to be construed as limiting the subject matter described.

The features of the present disclosure are set forth with particularity in the appended claims. A better understanding of the features and advantages of the present will be obtained by reference to the following detailed description that sets forth illustrative embodiments, in which the principles of the disclosure are utilized, and in view of the accompanying drawings as described herein below.

### Definitions

Unless defined otherwise, all technical and scientific terms used herein have the meaning commonly understood by a person of ordinary skill in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

A “microglial-like cell” is a cell derived from a stem cell that expresses a microglial phenotype. A microglial phenotype includes prolonged or transient expression of one or more microglial associated markers, including but not limited to Tmem119, P2Y12, and CD45. See also Speicher et al., Molec. Neurodegen. 14: 46, 2019. Microglia and microglial like cells express different phenotypes depending on whether or not they are activated/reactive.

As used herein “homeostatic microglia” or “ramified microglia” refers to a microglial cell that is found throughout the brain and spinal cord and can be transformed into its activated or reactive form at any time in response to injury, such as neuroinflammation. “Homeostatic microglia” may be used interchangeably with “ramified microglia.” Homeostatic microglia are typically characterized as small round cells with numerous branching processes, and containing minimal cytoplasm. Markers associated with homeostatic microglia include, but are not limited to, P2Y12+, Tmem119+, Siglech+, SPP1-, and CD68-.

“Activated microglia” may be used interchangeably with “reactive microglia.” Activated microglia are typically characterized as being rod-like, devoid of branching processes, and containing numerous lysosomes and phagosomes. Markers associated with active microglia include, but are not limited to, CD68+, SPP1+, Igf1+, Tmem119- and P2Y12-.

By “administer” or “administration” is meant giving, supplying, or dispensing a composition, agent, therapeutic and the like to a subject, or applying or bringing the composition and the like into contact with the subject. Administering or administration may be accomplished by any of a number of routes, such as, for example, without limitation, topical, oral, subcutaneous, intramuscular, intraperitoneal, intravenous (IV), injection, intrathecal, intramuscular, dermal, intradermal, intracranial, inhalation, rectal, intravaginal, or intraocular.

By “agent” is meant any small molecule chemical compound, cell, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “ameliorate” is meant decrease, reduce, delay diminish, suppress, attenuate, arrest, or stabilize the development or progression of a disease or pathological condition. Tuberous sclerosis is an exemplary disease or pathological condition.

By “analog” is meant a molecule that is not identical, but has analogous functional or structural features. For example, a polypeptide analog retains the biological activity of a corresponding naturally-occurring polypeptide, while having certain biochemical modifications that enhance the analog’s function relative to a naturally occurring polypeptide. Such biochemical modifications could increase the analog’s protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

As used herein, the terms “determining,” “assessing,” “assaying,” “measuring,” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “disease” is meant any condition or disorder that damages or interferes with the normal function of a cell, tissue, or organ. Examples of diseases include neuroinflammatory diseases, neurodegenerative diseases, and neuronal injury. Examples of diseases include but are not limited to Alzheimer’s disease, Parkinson’s disease, Huntington’s disease, multiple sclerosis (MS), amyotrophic lateral sclerosis (ALS), glaucoma, metachromatic leuokodystrophy, adrenoleukodystophy, lysosomal storage disorders, traumatic brain injury, spinal cord injury, spinal cord crush, and/or optic nerve injury, or a combination thereof.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

In some embodiments, an effective amount is the amount of one or more peptidase inhibitors (e.g., E64 and Serpina3n), treated microglia cells (e.g., adult and/or reactive microglia cells treated with one or more peptidase inhibitors), and/or neonatal microglia cells (e.g., neonatal microglia cells derived from stem cells or iPSCs) required to treat a disease or disorder (e.g., neuroinflammatory disease, neurodegenerative disease, and/or neuronal injury) or symptom thereof, in a subject. In some embodiments, an effective amount is the amount of one or more peptidase inhibitors (e.g., E64 and Serpina3n) required to inhibit reactive microglia gene expression and/or increase neonatal gene expression in a reactive microglia cell in a subject with a disease or disorder (e.g., neuroinflammatory disease, neurodegenerative disease, and/or neuronal injury) or symptom thereof. In some embodiments, an effective amount is the amount of one or more peptidase inhibitors (e.g., E64 and Serpina3n) required to decreases the expression of CD68 and/or SPP1 and increases the expression of P2Y12 and/or TMEM119 in a reactive microglia cell in a subject with a disease or disorder (e.g., neuroinflammatory disease, neurodegenerative disease, and/or neuronal injury) or symptom thereof. In some embodiments, an effective amount is the amount of one or more peptidase inhibitors (e.g., E64 and Serpina3n) required to decrease reactive microglia gene expression and/or increase neonatal gene expression in a reactive microglia cell in vitro. In some embodiments, an effective amount is the amount of one or more peptidase inhibitors (e.g., E64 and Serpina3n) required to decreases the expression of CD68 and/or SPP1 and increases the expression of P2Y12 and/or TMEM119 in a reactive microglia cell in vitro. In some embodiments, an effective amount of one or more peptidase inhibitors (e.g., E64 and Serpina3n) is the amount required to re-establish microglia homeostasis. In some embodiments, an effective amount of one or more peptidase inhibitors (e.g., E64 and Serpina3n) is the amount required to re-establish microglia homeostasis in vitro. In some embodiments, an effective amount of one or more peptidase inhibitors (e.g., E64 and Serpina3n) is the amount required to re-establish microglia homeostasis relative to a reference, e.g., reactive microglia or homeostatic microglia.

In some embodiments, an effective amount of a pharmaceutical composition is the amount required to promoting scar-free healing after neuronal injury. In some embodiments, an effective amount of a pharmaceutical composition is the amount required to promote scar-free healing after neuronal injury in a subject relative to a reference, e.g., untreated subject. In some embodiments, an effective amount of a pharmaceutical composition is the amount required to promote axon regeneration or regrowth. In some embodiments, an effective amount of a pharmaceutical composition is the amount required to promote axon regeneration or regrowth in a subject relative to a reference, e.g., untreated subject. In some embodiments, an effective amount of a pharmaceutical composition is the amount required to reduce neuroinflammation. In some embodiments, an effective amount of a pharmaceutical composition is the amount required to reduce neuroinflammation in a subject relative to a reference, e.g., untreated subject.

In some embodiments, an effective amount of a pharmaceutical composition is the amount required to increase fibronectin production in a microglia cell of a subject. In some embodiments, an effective amount of a pharmaceutical composition is the amount required to increase fibronectin production in a cell of a subject relative to a reference, e.g., untreated subject. In some embodiments, fibronectin production in a microglia cell is increased at least about 5%, at least about 10%, at least about 20%, at least about 30%, at least about 40%, at least about 50%, at least about 60%, at least about 70%, at least about 80%, at least about 90%, or at least about 100% relative to a reference, e.g., untreated subject.

The effective amount of a composition as used to practice the methods of therapeutic treatment of a disease, condition, or pathology, varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “induced pluripotent stem cell (iPSC)” is meant a cell derived from skin or blood cells that has been reprogrammed back into an embryonic-like pluripotent state to enable the development of an unlimited source of any type of cell needed for therapeutic purposes. In some embodiments, iPSCs are differentiated into microglial cells. Methods for differentiating microglial cells from iPSCs include those described in International Application Nos. PCT/US2018/019763 and PCT/US2016/039336, Abud et al., iPSC-Derived Human Microglia-like Cells to Study Neurological Diseases. Neuron 94, 278-293 (April 2017), and Muffat et al., Efficient derivation of microglia-like cells from human pluripotent stem cells. Nature Medicine, Vol. 22, No. 11 (November 2016), the entire contents of which are incorporated herein by reference.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by high-performance liquid chromatography (HPLC) analysis.

By “marker” is meant any clinical indicator, protein, metabolite, or polynucleotide having an alteration associated with a disease, disorder, or condition.

By “microglia” is meant an immune cell of myeloid lineage resident in the central nervous system.

By “neonatal” is meant a newborn subject less than four weeks of age. In some embodiments, the neonatal subject is a mammalian subject. In some embodiments, the neonatal subject is a human subject.

By “neonatal microglia cell” is meant a microglia cell that expresses neonatal markers. In some embodiments, the neonatal microglia cell is characterized as having one or more of the following gene expression profiles: CD68-, SPP1-, P2Y12+, and/or TMEM119+. In some embodiments, the neonatal microglia cell is characterized as secreting fibronectin and associated binding proteins. In some embodiments, the neonatal microglia cell is characterized as expressing a number of extracellular and intracellular peptidase inhibitors. In some embodiments, the neonatal microglia cell is obtained from a neonatal subject or donor. In some embodiments, the neonatal microglia cell is differentiated in vitro. In some embodiments, the neonatal microglia cell is differentiated from a stem cell. In some embodiments, the stem cell is a human stem cell. In some embodiments, the neonatal microglia cell is differentiated from an induced pluripotent stem cell (iPSC). In some embodiments, the iPSC is a human iPSC. Methods for differentiating neonatal microglial cells from iPSCs include those described International Application Nos. PCT/US2018/019763 and PCT/US2016/039336, Abud et al., iPSC-Derived Human Microglia-like Cells to Study Neurological Diseases. Neuron 94, 278-293 (April 2017), and Muffat et al., Efficient derivation of microglia-like cells from human pluripotent stem cells. Nature Medicine, Vol. 22, No. 11 (November 2016), the entire contents of which are incorporated herein by reference.

As used herein “neurodegenerative disease” refers to a disease or disorder characterized by the progressive loss of structure and/or function of neurons, including death of neurons. Exemplary neurodegenerative diseases include, without limitation, Alzheimer’s disease, Parkinson’s disease, glaucoma, metachromatic leuokodystrophy, adrenoleukodystophy, other lysosomal storage disorders, multiple sclerosis, and amyotrophic lateral sclerosis.

By “neuroinflammation” is meant an inflammatory response within the central nervous system, such as the brain or spinal cord.

As used herein “neuroinflammatory disease” refers to a disease or disorder where immune responses damage components of the nervous system. Neuroinflammatory diseases include many neurodegenerative disorders. It is also associated with aging and traumatic brain injury.

By “neuronal injury” is meant an injury to neurons. Some embodiments of neuronal injuries include, but are not limited to, traumatic brain injury, spinal cord injury, spinal cord crush, and optic nerve injury.

As used herein, “obtaining” as in “obtaining an agent” includes synthesizing, purchasing, or otherwise acquiring the agent.

By “increases” or “reduces” is meant a positive or negative alteration, respectively, of at least 10%, 25%, 50%, 75%, or 100%.

As used herein, “obtaining” as in “obtaining an agent” includes synthesizing, purchasing, or otherwise acquiring the agent.

“Patient” or “subject” as used herein refers to a subject, e.g., a mammalian subject diagnosed with or suspected of having or developing a disease or a disorder. In some embodiments, the term “patient” refers to a mammalian subject with a higher than average likelihood of developing a disease or a disorder. Exemplary patients can be humans, non-human primates, cats, dogs, pigs, cattle, cats, horses, goats, sheep, rodents (e.g., mice, rabbits, rats, or guinea pigs) and other mammalians that can benefit from the therapies disclosed herein. Exemplary human patients can be male and/or female. “Patient in need thereof” or “subject in need thereof” is referred to herein as a patient diagnosed with or suspected of having a disease or disorder.

By “peptidase inhibitor,” “proteinase inhibitor,” or “protease inhibitor” is meant an agent, compound, or substance that inhibits the activity of at least one peptidase. As used herein, “proteinase,” “peptidase” and “protease” may be used interchangeably. In some embodiments, the peptidase inhibitor is an inhibitor of one or more cysteine peptidases. In some embodiments, the peptidase inhibitor is an inhibitor of one or more serine proteases.

In some embodiments, the peptidase inhibitor is E64 (IUPAC Name: (1S,2S)-2-(((S)-1-((4-Guanidinobutyl)amino)-4-methyl-1-oxopentan-2-yl)carbamoyl)cyclopropanecarboxylic acid) or an analog thereof, which has the chemical formula C₁₅H₂₇N₅O₅. E64 is a membrane-permeable irreversible inhibitor of a wide range of cysteine peptidases. In some embodiments, E64 has the following chemical structure:

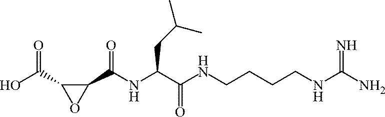 [SMILES: CC(C)C[C@H](NC(=O)[C@H]1O[C@@H]1C(=O)O)C(=O)NCCCCNC(=N)N]

In some embodiments, the peptidase inhibitor is Serpina3n. Serpina3n is a serine protease inhibitor. In some embodiments, Serpina3n is a polypeptide or fragment thereof having an amino acid sequence that is at least 85%, at least 90%, at least 95%, at least 98%, at least 99%, or 100% identical to a Serpina3n acid sequence associated with NCBI Reference Sequence: NP_033278.2. In some embodiments, Serpina3n is at least 85%, at least 90%, at least 95%, at least 98%, at least 99%, or 100% identical to the mTOR in Mus musculus. An exemplary Serpina3n full-length amino acid sequence is provided below:

1 MAFIAALGLL MAGICPAVLC FPDGTLGMDA AVQEDHDNGT QLDSLTLASI NTDFAFSLYK
61 ELVLKNPDKN IVFSPLSISA ALAVMSLGAK GNTLEEILEG LKFNLTETSE ADIHQGFGHL
121 LQRLNQPKDQ VQISTGSALF IEKRQQILTE FQEKARALYQ AEAFTADFQQ PRQAKKLIND
181 YVRKQTQGMI KELVSDLDKR TLMVLVNYIY FKAKWKVPFD PLDTFKSEFY AGKRRPVIVP
241 MMSMEDLTTP YFRDEELFCT VVELKYTGNA SAMFILPDQG KMQQVEASLQ PETLRKWKNS
301 LKPRMIDELH LPKFSISTDY SLEDVLSKLG IREVFSTQAD LSAITGTKDL RVSQVVHKAV
361 LDVAETGTEA AAATGVKFVP MSAKLYPLTV YFNRPFLIMI FDTETEIAPF IAKIANPK

In some embodiments, the peptidase inhibitor decreases expression of CD68. In some embodiments, the peptidase inhibitor decreases expression of SPP1. In some embodiments, the peptidase inhibitor decreases expression of CD68 and SPP1. In some embodiments the peptidase inhibitor increases expression of P2Y12. In some embodiments the peptidase inhibitor increases expression of TMEM119. In some embodiments the peptidase inhibitor increases expression of P2Y12 and TMEM119. In some embodiments, the peptidase inhibitor decreases expression of CD68 and increases expression of P2Y12. In some embodiments, the peptidase inhibitor decreases expression of CD68 and increases expression of TMEM119. In some embodiments, the peptidase inhibitor decreases expression of CD68 and increases expression of P2Y12 and TMEM119. In some embodiments, the peptidase inhibitor decreases expression of SPP1 and increases expression of P2Y12. In some embodiments, the peptidase inhibitor decreases expression of SPP1 and increases expression of TMEM119. In some embodiments, the peptidase inhibitor decreases expression of SPP1 and increases expression of P2Y12 and TMEM119. In some embodiments, the peptidase inhibitor decreases expression of CD68 and SPP1 and increases expression of P2Y12. In some embodiments, the peptidase inhibitor decreases expression of CD68 and SPP1 and increases expression of TMEM119. In some embodiments, the peptidase inhibitor decreases expression of CD68 and SPP1 and increases expression of P2Y12 and TMEM119.

In some embodiments, one or more peptidase inhibitors are administered to a microglia cell (e.g., reactive microglia cell). In some embodiments, the one or more peptidase inhibitors are administered to a microglia cell in vitro. In some embodiments, one or more peptidase inhibitors are administered to a microglia cell in a subject. In some embodiments, one or more peptidase inhibitors are administered to a reactive microglia cell in a subject. In some embodiments, one or more peptidase inhibitors are administered to a reactive microglial cell in vitro. In some embodiments, the one or more peptidase inhibitors are administered to a reactive microglia cell in a subject at the site of neuronal injury. In some embodiments, a microglia cell (e.g., reactive microglia cell) that has been treated with one or more peptidase inhibitors is administered to a subject. In some embodiments, the treated microglia cell (e.g., reactive microglia cell) is administered to a subject via injection and/or transplantation. In some embodiments, the treated microglia cell is derived from a subject. In some embodiments, the treated microglia cell is derived from a donor. In some embodiments, the subject is an adult subject or donor. In some embodiments, the treated reactive microglia cell has one or more of the following gene expression profiles: CD68+, SPP1+, P2Y12-, and/or TMEM119-. In some embodiments, the treated reactive microglia cell has persistent expression of Fn1 and no significant induction of proteinase inhibitors.

In some embodiments, one or more peptidase inhibitors increases fibronectin production in a microglia cell. In some embodiments, one or more peptidase inhibitors increases fibronectin production in a microglia cell of a subject relative to a reference, e.g., untreated cell. In some embodiments, one or more peptidase inhibitors increases fibronectin production in a microglia cell by at least about 5%, at least about 10%, at least about 20%, at least about 30%, at least about 40%, at least about 50%, at least about 60%, at least about 70%, at least about 80%, at least about 90%, or at least about 100% relative to a reference, e.g., untreated cell.

In some embodiments, one or more peptidase inhibitors are administered for the treatment of a neurodegenerative or a neuroinflammatory disease or disorder. In some embodiments, one or more peptidase inhibitors are administered for the treatment of neuronal injury. In some embodiments, one or more peptidase inhibitors are administered for promoting scar-free healing after neuronal injury. In some embodiments, one or more peptidase inhibitors are administered for promoting axon regeneration or regrowth. In some embodiments, one or more peptidase inhibitors are administered for decreasing neuroinflammation. In some embodiments, one or more peptidase inhibitors are administered for re-establishing microglia homeostasis.

By “phospholipase A2 inhibitor” is meant an agent, compound, or substance that inhibits the activity of a serine hydrolase enzyme. In some embodiments, a phospholipase A2 inhibitor is used in any of the pharmaceutical compositions or methods provided herein. In some embodiments, a microglial cell or microglial-like cell is treated with a phospholipase A2 inhibitor. In some embodiments, a microglial cell or microglial-like cell is treated with a phospholipase A2 inhibitor and a peptidase inhibitor.

In some embodiments, the phospholipase A2 inhibitor is Varespladib (LY315920) (IUPAC Name: 2-(1-benzyl-2-ethyl-3-oxamoylindol-4-yl)oxyacetic acid) or an analog thereof, which has the chemical formula C₂₁H₂₀N₂O₅. Varespladib is an inhibitor of the IIa, V, and X isoforms of secretory phospholipase A2 (sPLA2) and acts as an anti-inflammatory agent by disrupting the first step of the arachidonic acid pathway of inflammation. In some embodiments, Varespladib has the following chemical structure:

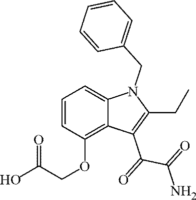 [SMILES: CCc1c(C(=O)C(N)=O)c2c(OCC(=O)O)cccc2n1Cc1ccccc1]

The term “pharmaceutically acceptable vehicle” refers to conventional carriers (vehicles) and excipients that are physiologically and pharmaceutically acceptable for use, particularly in mammalian, e.g., human, subjects. Such pharmaceutically acceptable vehicles are known to the skilled practitioner in the pertinent art and can be readily found in Remington’s Pharmaceutical Sciences, by E. W. Martin, Mack Publishing Co., Easton, Pa., 15th Edition (1975) and its updated editions, which describes compositions and formulations suitable for pharmaceutical delivery of one or more therapeutic or immunogenic compositions, such as one or more vaccines, and additional pharmaceutical agents. In general, the nature of a pharmaceutically acceptable carrier depends on the particular mode of administration being employed. For instance, parenteral formulations usually comprise injectable fluids/liquids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers may include, for example, pharmaceutical grades of mannitol, lactose, starch, or magnesium stearate, which typically stabilize and/or increase the half-life of a composition or drug. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the terms “prevent,” “preventing,” “prevention,” “prophylactic treatment,” and the like refer to reducing the probability of developing a disorder or condition in a subject, who does not have, but is at risk of or susceptible to developing a disorder or condition.

The terms “protein,” “peptide,” “polypeptide,” and their grammatical equivalents are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three (3) amino acids long. A protein, peptide, or polypeptide can refer to an individual protein or a collection of proteins. One or more of the amino acids in a protein, peptide, or polypeptide can be modified, such as glycoproteins, for example, by the addition of a chemical entity such as a carbohydrate group, a hydroxyl group, a phosphate group, a farnesyl group, an isofarnesyl group, a fatty acid group, a linker for conjugation, functionalization, or other modifications, etc. A protein, peptide, or polypeptide can also be a single molecule or can be a multi-molecular complex. A protein, peptide, or polypeptide can be just a fragment of a naturally occurring protein or peptide. A protein, peptide, or polypeptide can be naturally occurring, recombinant, or synthetic, or any combination thereof.

In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA or DNA. Any of the proteins provided herein can be produced by any method known in the art. For example, the proteins provided herein can be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

Conservative amino acid substitutions are those substitutions that, when made, least interfere with the properties of the original protein, that is, the structure and especially the function of the protein is conserved and is not significantly changed by such substitutions. Examples of conservative amino acid substitutions are known in the art, e.g., as set forth in, for example, U.S. Publication No. 2015/0030628. Conservative substitutions generally maintain (a) the structure of the polypeptide backbone in the area of the substitution, for example, as a sheet or helical conformation; (b) the charge or hydrophobicity of the molecule at the target site; and/or (c) the bulk of the side chain

The substitutions that are generally expected to produce the greatest changes in protein properties are non-conservative, for instance, changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

By “reduces” is meant a negative alteration of at least 5%, 10%, 25%, 30%, 40%, 50%, 75%, 80%, 85%, 90%, 95%, 98%, or 100%.

By “reference” is meant a standard or control condition.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those of ordinary skill in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 µg/ml denatured salmon sperm DNA (ssDNA). In a most preferred embodiment, hybridization will occur at 42° C. in 250 mM NaCl, 25 mM trisodium citrate, 1% SDS, 50% formamide, and 200 µg/ml ssDNA. Useful variations on these conditions will be readily apparent to those of ordinary skill in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those of ordinary skill in the art. Hybridization techniques are well known to those of ordinary skill in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

“Sequence identity” refers to the similarity between amino acid or nucleic acid sequences that is expressed in terms of the similarity between the sequences. Sequence identity is frequently measured in terms of percentage identity (or similarity or homology); the higher the percentage, the more similar the sequences are. Homologs or variants of a given gene or protein will possess a relatively high degree of sequence identity when aligned using standard methods. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. In addition, other programs and alignment algorithms are described in, for example, Smith and Waterman, 1981, Adv. Appl. Math. 2:482; Needleman and Wunsch, 1970, J. Mol. Biol. 48:443; Pearson and Lipman, 1988, Proc. Natl. Acad. Sci. U.S.A. 85:2444; Higgins and Sharp, 1988, Gene 73:237-244; Higgins and Sharp, 1989, CABIOS 5:151-153; Corpet et al., 1988, Nucleic Acids Research 16:10881-10890; Pearson and Lipman, 1988, Proc. Natl. Acad. Sci. U.S.A. 85:2444; and Altschul et al., 1994, Nature Genet. 6:119-129. The NCBI Basic Local Alignment Search Tool (BLAST™) (Altschul et al. 1990, J. Mol. Biol. 215:403-410) is readily available from several sources, including the National Center for Biotechnology Information (NCBI, Bethesda, Md.) and on the Internet, for use in connection with the sequence analysis programs blastp, blastn, blastx, tblastn and tblastx.

By “small hairpin RNA” or “shRNA” is meant an RNA sequence having a stem-loop structure. A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The term “hairpin” is also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches. The multiple stem-loop structures can be linked to one another through a linker, such as, for example, a nucleic acid linker, a miRNA flanking sequence, other molecule, or some combination thereof. While there may be some variation in range, a conventional stem-loop shRNA can comprise a stem ranging from 19 to 29 bp, and a loop ranging from 4 to 30 bp. “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. In some embodiments, the precursor miRNA molecule can include more than one stem-loop structure. MicroRNAs are endogenously encoded RNA molecules that are about 22-nucleotides long and generally expressed in a highly tissue- or developmental-stage-specific fashion and that post-transcriptionally regulate target genes. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Thus, one can design and express artificial miRNAs based on the features of existing miRNA genes.

By “subject” is meant an animal, e.g., a mammal, including, but not limited to, a human, a non-human primate, or a non-human mammal, such as a bovine, equine, canine, ovine, or feline mammal, or a sheep, goat, llama, camel, or a rodent (rat, mouse), gerbil, or hamster. In particular aspects as described herein, the subject is a human subject, such as a patient.

Ranges provided herein are understood to be shorthand for all of the values within the range, inclusive of the first and last stated values. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater, consecutively, such as to 100 or greater.

As used herein, the terms “treat,” “treating,” “treatment,” and the like refer to reducing, diminishing, decreasing, delaying, abrogating, ameliorating, or eliminating, a disease, condition, disorder, or pathology, and/or symptoms associated therewith. While not intending to be limiting, “treating” typically relates to a therapeutic intervention that occurs after a disease, condition, disorder, or pathology, and/or symptoms associated therewith, have begun to develop to reduce the severity of the disease, etc., and the associated signs and symptoms. It will be appreciated that, although not precluded, treating a disorder or condition does not require that the disease, condition, disorder, pathology, or the symptoms associated therewith, be completely eliminated.

Unless specifically stated or obvious from context, as used herein, the term “or” is understood to be inclusive. Unless specifically stated or obvious from context, as used herein, the terms “a,” “an,” and “the” are understood to be singular or plural. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1F depict scar-free wound healing after neonatal spinal cord crush injury. FIG. 1A are images depicting sagittal spinal cord sections at 2 weeks after P2 crush (top) or adult crush (bottom) stained with anti-5HT antibody. Stars indicate the lesion site. Scale bar: 500 µm. FIG. 1B is a graph depicting the quantification of serotonergic axon density (normalized to proximal of lesion) in spinal cord distal to the lesion site at 2 weeks post crush. Two-way ANOVA followed by post hoc Bonferroni correction. *p < 0.05; ***p < 0.001. n=8, 5, 5 and 8 for P2 crush, P7 crush, P20 crush and adult crush respectively. FIG. 1C are images depicting spinal sagittal sections at 10 weeks after P2 crush (top) or adult crush (bottom) stained with corticospinal axon tracing by AAV-ChR2-mCherry. Stars indicate the lesion site. Scale bar: 1 mm. FIG. 1D is a graph depicting the quantification of corticospinal axon density (normalized to proximal of lesion) in spinal cord distal to the lesion site at 10 weeks post crush. Two-way ANOVA followed by post hoc Bonferroni correction. ***p < 0.001. n=5 per group. FIG. 1E are images depicting sagittal spinal cord sections at 2 weeks after P2 crush (top) or adult crush (bottom) stained with antibodies against Collagen I, Fibronectin, CD68, GFAP, P2Y12 and CD31, respectively. Scale bar: 250 µm. FIG. 1F are graphs depicting the quantification of Collagen I, Fibronectin, CD68, GFAP, P2Y12 and CD31 immunoreactive intensity (normalized to the intact region) in the lesion site at 2 weeks after P2 or adult crush. Student’s t test (two-tailed, unpaired). ***p < 0.001. n=8 per group.

FIGS. 2A-2E depict age-dependent decline in serotonergic axon regrowth and wound healing. FIG. 2A are images depicting the spinal cord sagittal sections showing 5-HT-labeled axons from sham, P7 or P20 mice at 2 weeks after crush. Scale bar: 500 µm. FIG. 2B are images depicting the spinal cord sections from sham, P7 or P20 mice at 2 weeks after crush stained with antibodies against Collagen I, CD68, P2Y12 or CD31. Scale bar: 250 µm. FIG. 2C are images depicting spinal sagittal sections at 4 weeks after P2 crush (bottom) or sham (top) stained with corticospinal axon tracing by AAV-ChR2-mCherry. Stars indicate the lesion site. Scale bar: 500 µm. FIG. 2D are images depicting sagittal spinal cord sections at 2 weeks after P2 crush (top) or adult crush (bottom) stained with antibodies against Laminin, CSPG and GFAP, respectively. Scale bar: 250 µm. FIG. 2E are images depicting sagittal spinal cord sections at 2 weeks after P20 crush stained with antibodies against Laminin, CSPG and 5-HT, respectively. Scale bar: 250 µm.

FIGS. 3A-3G depict that microglia are required for bridge formation and rapid healing after neonatal spinal cord crush. FIG. 3A are images depicting spinal lesions after P2 crush stained with antibodies against fibronectin, 5-HT and DAPI. Fibronectin is transiently highly expressed in the cells around the lesion at 3 dpi. Wound heals with serotonergic axons crossing at 7 dpi. Scale bar: 50 µm. FIG. 3B are images depicting spinal cord lesion at different time points after injury stained with antibodies against P2Y12, SPP1 and DAPI in Cx3cr1-GFP mice. Initially activated microglia lose the expression of P2Y12 and start to express SPP1 at 2 dpi. Microglia co-stained with P2Y12 and SPP1 accumulate in the lesion site, peaking around 3 dpi. Microglia transform into homeostatic stage with low-expression of SPP1 and highly ramified morphology at 7 dpi. Scale bar: 50 µm. FIG. 3C are images depicting spinal cord sagittal sections at 3 dpi from different groups of mice stained with antibodies against fibronectin, CD68 and DAPI. Microglia depletion with PLX3397 or Csf1r knockout leads to the defects of bridge formation at 3 dpi. Scale bar: 50 µm. FIG. 3D are graphs depicting the quantification of CD68 (left) and Fibronectin (right) immunoreactive intensity (normalized to the intact region) in the lesion site at 3 days after P2 crush. One-way ANOVA followed by post hoc Bonferroni correction. **p < 0.01. ***p < 0.001. n=5 per group. FIG. 3E are images depicting spinal cord sagittal sections at 7 dpi in different groups of P2 crushed mice stained with antibodies against 5-HT, GFAP and DAPI. Microglia depletion with PLX3397 or Csf1r knockout impairs wound healing and axon regeneration after neonatal crush. Scale bar: 250 µm. FIG. 3F is a graph depicting the quantification of GFAP immunoreactive intensity (normalized to the intact region) in the lesion site at 7 days after P2 crush. One-way ANOVA followed by post hoc Bonferroni correction. ***p <0.001. n=5 per group. FIG. 3G is a graph depicting the quantification of serotonergic axons density (normalized to proximal of lesion) in spinal cord distal to the lesion site at 2 weeks after P2 crush. Two-way ANOVA followed by post hoc Bonferroni correction. ***p < 0.001. n=5 per group.

FIGS. 4A-4C depict distinct microglia/macrophage responses after neonatal or adult spinal cord crush. FIG. 4A are images depicting spinal cord sections stained with antibodies against CD68 and P2Y12 mice at 3 dpi, 7 dpi or 14 dpi. Higher magnification images showing P2Y12+ cells were co-labeled with CD68 at 3 dpi. Cells with highly ramified morphology at 7 dpi, and 14 dpi, around lesion sites. Scale bar: 100 µm. FIG. 4B are high magnification images from FIG. 3A depicting that CD68+ cells and Fibronectin matrix form bridges between gap at 3 dpi. Scale bar: 50 µm. FIG. 4C are images depicting immunolabeling for CD68 and P2Y12 in adult mice at 3 dpi, 7 dpi or 14 dpi showing CD68-positive cells lacking P2Y12 expression. Scale bar: 200 µm.

FIGS. 5A-5I depict scRNA-seq analysis of microglia isolated from lesion site after P2 injury. FIG. 5A is a tSNE plot depicting 5 clusters of microglia (MG0-MG4) isolated at different time points (0, 3 and 5 dpi) after P2 spinal cord crush. FIG. 5B is a heatmap depicting the top 15 markers for each individual cluster. FIG. 5C is a bar plot of different clusters of microglia at different time points after P2 injury. FIG. 5D depicts UMAPs of different clusters of microglia at different time points after P2 injury. FIG. 5E depicts violin plots showing high-level expression of Ms4a7 and Thbs1 in MG3 and Fn1 in both MG1 and MG3. FIG. 5F are images depicting RNA in situ hybridization showing Ms4a7 and Thbs1 enrichment in the epicenter of the lesion site and expression of Fn1 in and around the lesion site. Scale bar: 200 µm. FIG. 5G are high magnification images from FIG. 5F depicting co-expression of Ms4a7 and P2ry12 in microglia in the lesion site. Scale bar: 20 µm. FIG. 5H is a schematic drawing depicting the distribution of MG1 and MG3 microglia with MG3 abundance in the lesion and at the bridge and MG1 around the lesion. FIG. 5I is a graph depicting selected GO terms and associated genes enriched in cluster 3 microglia (MG3).

FIGS. 6A-6B depict histological assessments of bridges formed after neonatal spinal cord crush. FIG. 6A are high magnification images depicting spinal sections of spinal cord bridges area stained with antibodies against Fibronectin, GFAP, P2Y12, Collagen I and DAPI at 3 dpi in P2 injury. Scale bar: 50 µm. FIG. 6B are images depicting spinal sections of Cx3cr1-GFP mice immunolabeled with Caspase-3 showing cells around the lesion sites at 3 dpi in P2 injury. Scale bar: 200 µm.

FIGS. 7A-7E depict conditional deletion of fibronectin from microglia impaired wound healing and axon regrowth after P2 spinal cord crush. FIG. 7A are images depicting spinal cord sections stained with antibodies against CD68 and fibronectin taken from 3 dpi of control (Fn1f/f) or different Fn1 conditional knockout (Cx3cr1-Cre; Fn1f/f, Tie2-Cre; Fn1f/f, Alb-Cre; Fn1f/f) mice at 3 dpi. Scale bar 250 µm. FIG. 7B is a graph depicting the quantification of Fibronectin intensity in the lesion site (3 dpi in P2 injury) in different groups of mice. One-way ANOVA followed by post hoc Bonferroni correction. **p < 0.01. n.s., not significant. n=3 per group. FIG. 7C are images depicting spinal sections stained with antibodies against 5-HT or GFAP, or P2Y12 taken in control (Fn1f/f) or different Fn1 conditional knockout (Cx3cr1-Cre; Fn1f/f, Tie2-Cre; Fn1f/f, Alb-Cre; Fn1f/f) mice at 14 dpi. Scale bar 250 µm. FIG. 7D is a graph depicting the quantification of serotonergic axons density (normalized to proximal of lesion) in the distal of lesion site at 2 weeks after P2 crush. Two-way ANOVA followed by post hoc Bonferroni correction. ***p < 0.001. n=5 per group. FIG. 7E are high magnification images from FIG. 7C depicting 5-HT axon terminals and GFAP+ astrocytes in the lesion site. Scale bar: 50 µm.

FIGS. 8A-8B depict that infiltrated CCR2+ monocytes/macrophages were eliminated after neonatal, but not adult spinal cord injury. Images of sagittal sections of injured spinal cord of Ccr2-RFP mice at 3 or 14 dpi are depicted for P2 crush (FIG. 8A) or adult crush (FIG. 8B). Sections were immunostained for CD68 and RFP (for Ccr2-RFP). Scale bar: 250 µm.

FIGS. 9A-9F depict that transplanted neonatal or proteinase inhibitor-treated adult microglia improved wound healing and axon regeneration. FIGS. 9A and 9B depict images of spinal cord sections at 2 weeks after adult spinal cord injury without transplantation (control), with adult microglia and vehicle (Vehicle) transplantation, with E64/SerpinA-treated adult microglia (Combination), and with postnatal day 1 microglia (P1 microglia) transplantation. Sections stained with antibodies against P2Y12, GFP (microglia were isolated from Cx3cr1-GFP mice) and CD68 (FIG. 9A) or GFP, Collagen I and Ly6G (FIG. 9B). Scale bar: 250 µm. FIGS. 9C and 9D are graphs depicting the quantification of the results from FIGS. 9A, 9B and 15C. While all transplantation groups exhibited reduced Ly6G, many microglia in P1 microglia and combination groups became P2Y12+ and CD68- in the lesions accompanied with reduced collagen and CSPG deposition in the lesion area. One-way ANOVA followed by post hoc Bonferroni correction. *p < 0.05, **p < 0.01, ***p < 0.001. n.s., not significant. n=6 per group. FIG. 9E are images depicting sagittal spinal cord sections from different groups of mice at 4 weeks after adult crush stained with anti-5-HT. Stars indicate the lesion site. Scale bar: 250 µm. FIG. 9F is a graph depicting the quantification of serotonergic axon density (normalized to proximal of lesion) in spinal cord distal to the lesion site at 4 weeks after crush and microglia transplantation in different groups of mice. Two-way ANOVA followed by post hoc Bonferroni correction. **p < 0.01; ***p < 0.001. n=7 per group.

FIGS. 10A-10D depict microglia depletion impaired wound healing and axon regrowth after neonatal spinal cord injury. FIG. 10A are images depicting P2Y12-stained spinal cord showing PLX3397-mediated depletion of microglia cells (left), and a graph depicting the quantification of microglia depletion in the spinal cord treated with PLX3397 or vehicle at 0, 7 or 14 dpi (right). Student’s t test (two-tailed, unpaired). ***p < 0.001. Scale bar: 250 µm. n=3, 5 and 5 for 0 dpi, 7 dpi and 14 dpi respectively. FIG. 10B are images and a graph depicting P2Y12-stained spinal cord sections from control (Csf1rf/f) and Csf1r KO (Cx3cr1-Cre; Csf1rf/f) mice showing ~70% reduction of microglia throughout the spinal cord in the mutant mice. Student’s t test (two-tailed, unpaired). ***p < 0.001. Scale bar: 250 µm. n=5 per group. FIG. 10C are images depicting sagittal spinal sections taken at 14 days after P2 crush and immunostained with antibodies against 5-HT, GFAP, laminin, CSPG or CD31. Scale bar: 200 µm. FIG. 10D are high magnification images from FIG. 10C showing 5-HT axon and GFAP+ astrocytes in the lesion site. Scale bar: 50 µm.

FIGS. 11A-11E depict isolation and scRNA-seq results of immune cells after neonatal spinal cord injury. FIG. 11A are FACS plots depicting selection of CD11b+, CD45+ cells from neonatal spinal cord dissociated cells. FIG. 11B is a tSNE plot depicting 14 clusters and population annotations. FIGS. 11C and 11D depict relative proportions of microglia among total cells (FIG. 11C) and dividing microglia among microglia cells (FIG. 11D). FIG. 11E is a table depicting the percentage of each cluster and their signature genes (left) and a heatmap depicting top 30 DE (differential expression) genes for each of the 14 clusters (right). FIG. 12 are plots depicting differentially expressed genes in different clusters.

FIGS. 13A-13B are dot plots depicting gene expression correlation between proliferative-region associated microglia (PAM) (FIG. 13A) or disease associated microglia (DAM) (FIG. 13B) (normalized to homeostatic microglia) with MG3 (normalized to MG0). Different sets of up- and down-regulated genes are depicted.

FIGS. 14A-14C depict network analysis and further characterization of MG3 DE genes. FIG. 14A is a diagram depicting correlated gene modules that underlie cluster identities of MG3 microglia. FIGS. 14B and 14C depict the expression of selected genes in microglia isolated at different time points after adult injury using bulk RNAseq. Expression of genes associated with endopeptidase inhibitor activity (FIG. 14B), and extracellular matrix (FIG. 14C), in adult microglia at 0, 3 and 5 dpi. One-way ANOVA followed by post hoc Bonferroni correction. *p < 0.05; **p< 0.01. n.s., not significant. n=3 per group.

FIGS. 15A-15C depict microglia isolation and transplantation. FIG. 15A are images (left) and a graph (right) depicting isolated microglia (P2Y12+) from neonatal or adult Cx3cr1-GFP mice. Scale bar: 50 µm. FIG. 15B are images depicting spinal cord sections showing the activation of transplanted microglia in the adult lesion at 2 days after grafting. Scale bar: 250 µm. FIG. 15C are images depicting spinal cord sections showing the GFAP and CSPG in the adult lesion at 14 days after grafting. Scale bar: 250 µm.

### DETAILED DESCRIPTION OF THE INVENTION

Provided herein are methods for reducing neuroinflammation. The methods provided herein may also be used for the treatment of neurodegenerative diseases and disorders. The methods provided herein use one or more peptidase inhibitors (e.g., E64 and Serpina3n), microglia-like cells (e.g., microglia cells treated with one or more peptidase inhibitors), and/or neonatal microglia cells (e.g., neonatal microglia cells derived from stem cells or iPSCs), or pharmaceutical compositions thereof. The invention also provides methods for reducing post-injury scar formation in the central nervous system.

As reported in detail below, the invention is based, at least in part, on the discovery that proteinase inhibitors are useful for reducing neuroinflammation and in the treatment of neurodegenerative diseases, and other diseases associated with chronically reactive microglia.

### Characterizing Diseases with Reactive Microglia in a Subject

The present disclosure features methods that are useful for the treatment of neuroinflammatory diseases or disorders, neurodegenerative diseases or disorders, and/or neuronal injury. In particular, the disclosure features methods that are useful for neuroinflammatory diseases or disorders, neurodegenerative diseases or disorders, and/or neuronal injury that are characterized by the presence of reactive microglia. The present disclosure provides methods of treating disease and/or disorders or symptoms thereof which comprise administering a therapeutically effective amount of an agent or pharmaceutical composition comprising an agent herein to a subject (e.g., a mammal, such as a human). Thus, one embodiment is a method of treating a subject suffering from or susceptible to a neuroinflammatory disease or disorder, neurodegenerative disease or disorder, and/or neuronal injury, or symptom thereof.

In characterizing neuroinflammatory diseases or disorders, neurodegenerative diseases or disorders, and/or neuronal injury, the gene expression profile and/or morphology of microglia from a biological sample (e.g., cerebrospinal fluid (CSF), blood, plasma, serum) of a subject may be analyzed (e.g., a mammal, such as a human) to determine the presence of reactive or activated microglia cells. The morphology of reactive microglia is typically characterized as being rod-like, devoid of branching processes, and containing numerous lysosomes and phagosomes. The markers profile associated with reactive microglia include, but is not limited to: CD68+, SPP1+, Igf1+, Tmem119- and P2Y12-.

In some embodiments, the expression of one or more reactive microglia markers (e.g., CD68) are measured in microglia cells from a biological sample from a patient having a neuroinflammatory disease, neurodegenerative disease, and/or neuronal injury relative to the level of expression in a reference sample (i.e., a patient that does not have the disease characterized by reactive microglia). In some embodiments, the expression of one or more reactive microglia markers (e.g., CD68) are measured in microglia cells from a biological sample from a patient having a neuroinflammatory disease, neurodegenerative disease, and/or neuronal injury relative to the level of expression of a neonatal or homeostatic microglia cell. Polypeptide or polynucleotide fold change values may be determined using any method known in the art, including but not limited to quantitative PCR, RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, ELISA, microarray analysis, colorimetric assays, mass spectrometry (e.g., laser desorption/ionization mass spectrometry), fluorescence (e.g. sandwich immunoassay), surface plasmon resonance, ellipsometry, and atomic force microscopy.

The therapeutic methods of the disclosure in general comprise administration of an effective amount of the compounds herein, such as a pharmaceutical composition herein, to a subject (e.g., animal, human) in need thereof, including a mammal, particularly a human, to treat the disease or disorder (e.g., neuroinflammatory disease, neurodegenerative disease, and/or neuronal injury) or symptom thereof, under conditions such that the disease or disorder is treated to produce such effect. Such treatment will be suitably administered to subjects, particularly humans, suffering from, having, susceptible to, or at risk for a disease, disorder, or symptom thereof. Determination of those subjects in need of such treatment can be made by any objective or subjective determination by a diagnostic test or opinion of a subject or health care provider (e.g., genetic test, enzyme or protein marker, family history, and the like). The compounds herein may be also used in the treatment of any other diseases or disorders in which reactive microglia may be implicated.

In some embodiments, the present disclosure features methods of treating a neuroinflammatory disease or disorder, and symptoms thereof. Neuroinflammatory diseases or disorders occur when immune responses damage components of the nervous system, such as the brain and/or spinal cord. Neuroinflammatory diseases include many neurodegenerative disorders. Neuroinflammatory diseases are also associated with aging and traumatic brain injury. In some embodiments, the neuroinflammatory disease is selected from Alzheimer’s disease, Parkinson’s disease, and/or multiple sclerosis, or a combination thereof.

In some embodiments, the present disclosure features methods of treating a neurodegenerative disease or disorder, and symptoms thereof. Neurodegenerative diseases are characterized by the progressive loss of structure and/or function of neurons, including death of neurons. In some embodiments, the neurodegenerative disease is selected from Alzheimer’s disease, Parkinson’s disease, glaucoma, metachromatic leuokodystrophy, adrenoleukodystophy, lysosomal storage disorders, and/or amyotrophic lateral sclerosis, or a combination thereof.

In some embodiments, the present disclosure features methods of treating a neuronal injury, and symptoms thereof. Neuronal injury results in an injury to neurons. For example, spinal cord injury disrupts axonal connections between the brain and the spinal cord. A spinal cord injury triggers the formation of a scar, consisting of multiple cell types such as reactive astrocytes, fibroblasts, and microglia/macrophages, resulting in no spontaneous axon regeneration. Some embodiments of neuronal injuries include, but are not limited to, traumatic brain injury, spinal cord injury, spinal cord crush, and optic nerve injury, or a combination thereof.

In some embodiments, the present disclosure features methods of decreasing neuroinflammation. Neuroinflammation is characterized as an inflammatory response within the brain or spinal cord. Neuroinflammation may be the cause of or symptom of a neuroinflammatory disease, neurodegenerative disease, or neuronal injury.

### Peptidase Inhibitors

The present disclosure features one or more peptidase inhibitors (e.g., E64 and Serpina3n). The one or more one or more peptidase inhibitors may be used in any of the methods or combination therapy methods of the present invention. A peptidase inhibitor as described herein is an agent, compound, or substance that inhibits the activity of at least one peptidase.

In some embodiments, the one or more one or more peptidase inhibitors is a serine peptidase inhibitor. In some embodiments, the serine peptidase inhibitor is Serpina3n, or an analog thereof. In some embodiments, Serpina3n is a polypeptide or fragment thereof having an amino acid sequence that is at least 85%, at least 90%, at least 95%, at least 98%, at least 99%, or 100% identical to a Serpina3n acid sequence associated with NCBI Reference Sequence: NP_033278.2. In some embodiments, Serpina3n is at least 85%, at least 90%, at least 95%, at least 98%, at least 99%, or 100% identical to the mTOR in Mus musculus.

In some embodiments, the one or more peptidase inhibitors is a cysteine peptidase inhibitor. In some embodiments, the cysteine peptidase inhibitor is E64, or an analog thereof, which has the chemical formula C₁₅H₂₇N₅O₅. E64 is a membrane-permeable irreversible inhibitor of a wide range of cysteine peptidases. In some embodiments, E64 has the following chemical structure:

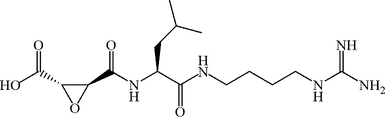 [SMILES: CC(C)C[C@H](NC(=O)[C@H]1O[C@@H]1C(=O)O)C(=O)NCCCCNC(=N)N]

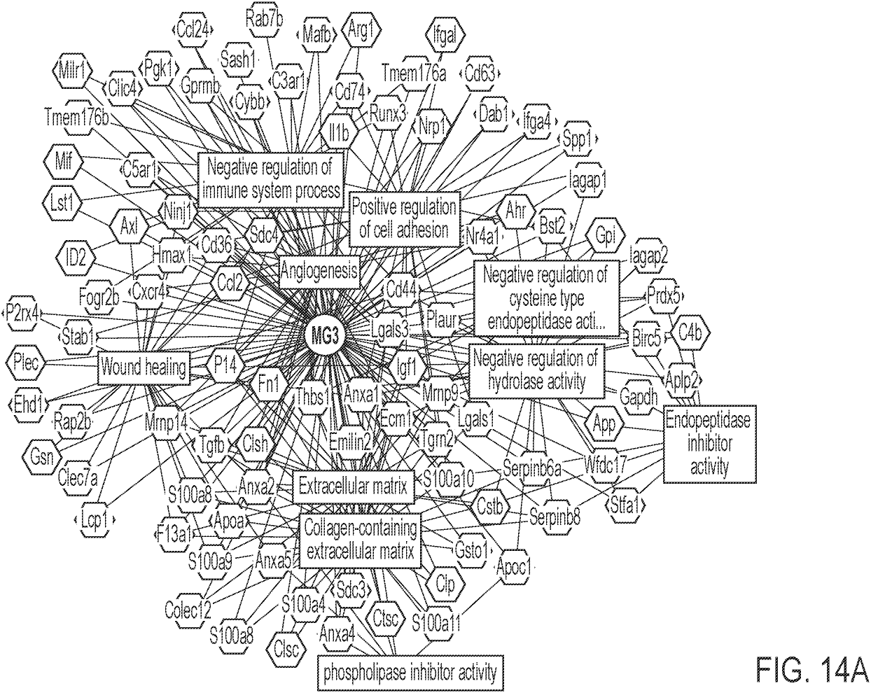

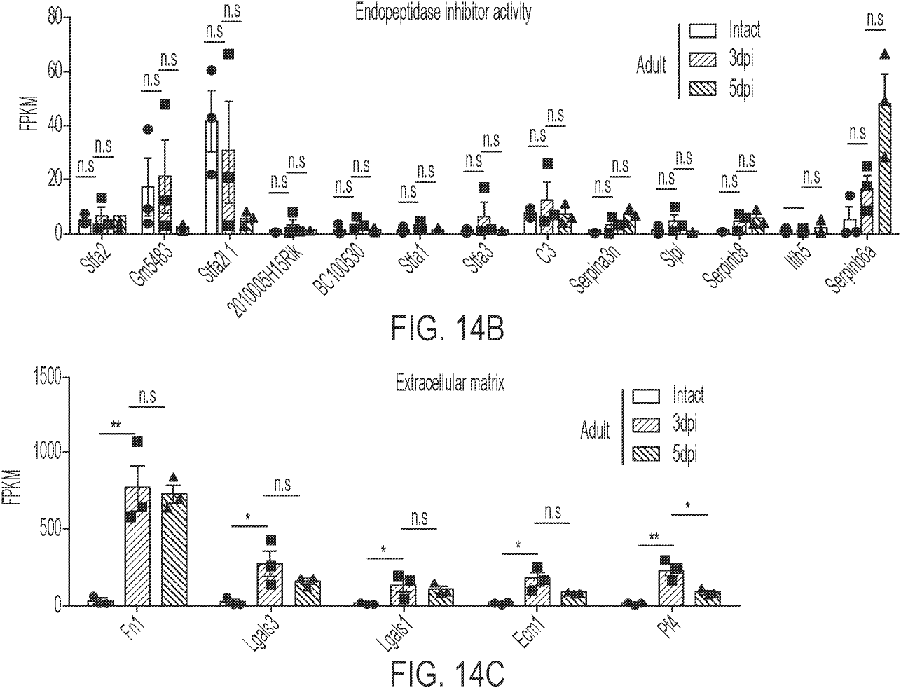

In some embodiments, the peptidase inhibitors include both a serine peptidase inhibitor and a cysteine peptidase inhibitor. In some embodiments, the peptidase inhibitors include both E64 and Serpina3n, or analogs thereof. In some embodiments, the one or more peptidase inhibitor is an inhibitor identified in FIGS. 14A-14C. In some embodiments, the peptidase inhibitor decreases expression of CD68. In some embodiments, the peptidase inhibitor decreases expression of SPP1. In some embodiments, the peptidase inhibitor decreases expression of CD68 and SPP1. In some embodiments the peptidase inhibitor increases expression of P2Y12. In some embodiments the peptidase inhibitor increases expression of TMEM119. In some embodiments the peptidase inhibitor increases expression of P2Y12 and TMEM119. In some embodiments, the peptidase inhibitor decreases expression of CD68 and increases expression of P2Y12. In some embodiments, the peptidase inhibitor decreases expression of CD68 and increases expression of TMEM119. In some embodiments, the peptidase inhibitor decreases expression of CD68 and increases expression of P2Y12 and TMEM119. In some embodiments, the peptidase inhibitor decreases expression of SPP1 and increases expression of P2Y12. In some embodiments, the peptidase inhibitor decreases expression of SPP1 and increases expression of TMEM119. In some embodiments, the peptidase inhibitor decreases expression of SPP1 and increases expression of P2Y12 and TMEM119. In some embodiments, the peptidase inhibitor decreases expression of CD68 and SPP1 and increases expression of P2Y12. In some embodiments, the peptidase inhibitor decreases expression of CD68 and SPP1 and increases expression of TMEM119. In some embodiments, the peptidase inhibitor decreases expression of CD68 and SPP1 and increases expression of P2Y12 and TMEM119.

In some embodiments, one or more peptidase inhibitors increases expression of Fnl and/or increases fibronectin production by the microglia cell. In some embodiments, one or more peptidase inhibitors increases fibronectin production in a microglia cell of a subject relative to a reference, e.g., untreated cell. In some embodiments, one or more peptidase inhibitors increases fibronectin production in a microglia cell by at least about 5%, at least about 10%, at least about 20%, at least about 30%, at least about 40%, at least about 50%, at least about 60%, at least about 70%, at least about 80%, at least about 90%, or at least about 100% relative to a reference, e.g., untreated cell.

In some embodiments, one or more one or more peptidase inhibitors are used in methods for treating a subject with a neuroinflammatory disease characterized by the presence of reactive microglia by administering an effective amount of the one or more peptidase inhibitors to a reactive microglia cell in the subject. In some embodiments, one or more peptidase inhibitors are used in methods for treating a subject with a neurodegenerative disease characterized by the presence of reactive microglia by administering an effective amount of the one or more peptidase inhibitors to a reactive microglia cell in the subject. In some embodiments, one or more peptidase inhibitors are used in methods for treating neuronal injury by administering an effective amount of the one or more peptidase inhibitors to a reactive microglia cell at the site of neuronal injury. In some embodiments, one or more peptidase inhibitors are used in methods for promoting scar-free healing after neuronal injury by administering an effective amount of the one or more peptidase inhibitors to a reactive microglia cell at the site of neuronal injury. In some embodiments, one or more peptidase inhibitors are used in methods for promoting axon regeneration or regrowth by administering an effective amount of the one or more peptidase inhibitors to a reactive microglia cell at the site in need of axon regeneration or regrowth. In some embodiments, one or more peptidase inhibitors are used in methods for decreasing neuroinflammation by administering an effective amount of the one or more peptidase inhibitors to a reactive microglia cell at the site of neuroinflammation. In some embodiments, one or more peptidase inhibitors are used in methods for re-establishing microglia homeostasis by treating a reactive microglia cell with the one or more peptidase inhibitors. In some embodiments, the one or more peptidase inhibitors as used herein is in vitro methods.

In some embodiments, the one or more peptidase inhibitors (e.g., E64 and Serpina3n) are used in combination with a treated microglia cell (e.g., reactive microglia cells treated with one or more peptidase inhibitors). In some embodiments, the one or more peptidase inhibitors (e.g., E64 and Serpina3n) are used in combination with a neonatal microglia cell (e.g., neonatal microglia cell derived from a stem cell or iPSC). In some embodiments, the one or more peptidase inhibitors (e.g., E64 and Serpina3n) are used in combination with a treated microglia cell (e.g., reactive microglia cells treated with one or more peptidase inhibitors) and a neonatal microglia cell (e.g., neonatal microglia cell derived from a stem cell or iPSC). In some embodiments, the treated microglia cell (e.g., reactive microglia cells treated with one or more peptidase inhibitors) is used in combination with a neonatal microglia cell (e.g., neonatal microglia cell derived from a stem cell or iPSC).

In some embodiments, one or more peptidase inhibitors are administered to a microglia cell (e.g., reactive microglia cell). In some embodiments, the one or more peptidase inhibitors are administered to a microglia cell in vitro. In some embodiments, one or more peptidase inhibitors are administered to a microglia cell in a subject. In some embodiments, one or more peptidase inhibitors are administered to a reactive microglia cell in a subject. In some embodiments, one or more peptidase inhibitors are administered to a reactive microglial cell in vitro. In some embodiments, the one or more peptidase inhibitors are administered to a reactive microglia cell in a subject at the site of neuronal injury. In some embodiments, a microglia cell (e.g., reactive microglia cell) that has been treated with one or more peptidase inhibitors is administered to a subject. In some embodiments, the treated microglia cell (e.g., reactive microglia cell) is administered to a subject via injection and/or transplantation. In some embodiments, the treated microglia cell is derived from a subject. In some embodiments, the treated microglia cell is derived from a donor. In some embodiments, the subject is an adult subject or donor. In some embodiments, the reactive microglia cell has one or more of the following gene expression profiles: CD68+, SPP1+, P2Y12-, and/or TMEM119-. In some embodiments, reactive microglia have persistent expression of Fnl and no significant induction of proteinase inhibitors.

The one or more peptidase inhibitors (e.g., E64 and Serpina3n) of the disclosure may be incorporated into a pharmaceutical composition as provided herein for administration to a subject. In some embodiments, one or more peptidase inhibitors (e.g., E64 and Serpina3n) are formulated for administration to a subject in need. In some embodiments, one or more peptidase inhibitors (e.g., E64 and Serpina3n) are incorporated into a pharmaceutical composition for administration to a subject. In some embodiments, one or more peptidase inhibitors (e.g., E64 and Serpina3n) are administered to a subject in need thereof to treat a disease or disorder (e.g., neuroinflammatory disease, neurodegenerative disease, and/or neuronal injury) and symptoms thereof. Pharmaceutical compositions as provided herein for administration to a subject may be formulated to one or more peptidase inhibitors (e.g., E64 and Serpina3n).

The therapeutic methods of the disclosure in general comprise administration of an effective amount of the one or more peptidase inhibitors (e.g., E64 and Serpina3n) described herein, such as a pharmaceutical composition thereof, to a subject (e.g., animal, human) in need thereof, including a mammal, particularly a human, to treat the disease or disorder or symptom thereof, under conditions such that the disease or disorder is treated to produce such effect. Such treatment will be suitably administered to subjects, particularly humans, suffering from, having, susceptible to, or at risk for a disease, disorder, or symptom thereof. Determination of those subjects in need of such treatment can be made by any objective or subjective determination by a diagnostic test or opinion of a subject or health care provider (e.g., genetic test, enzyme or protein marker, family history, and the like). The one or more peptidase inhibitors (e.g., E64 and Serpina3n) herein may be also used in the treatment of any other disorders in which reactive microglia may be implicated.

### Treated Microglia Cells

The present disclosure features treated microglia cells (e.g., microglia cells treated with one or more peptidase inhibitors). Treated microglia cells as described herein may be used in any of the methods or combination therapy methods of the present invention. In some embodiments, a treated microglia cell as described herein is a microglial cell or a microglial-like cell.

In some embodiments, the microglial cell or microglial-like cell expresses a SPP1 and/or a CD68 polypeptide or a polynucleotide encoding said polypeptide. In some embodiments, the microglial cell or microglial-like cell fails to express or expresses reduced levels of a P2Y12 polypeptide or a polynucleotide encoding said polypeptide. In some embodiments, the microglial cell or microglial-like cell has an ameboid morphology. In some embodiments, the microglial cell or microglial-like cell expresses a polypeptide selected from the group consisting of Igf1, Ms4a7, Fabp5 Mif Ms4a7, Thbs1, Clec7a, Ms4a7, Ms4a6c, Lgals1, fibronectin 1 (Fn1), thrombospondin 1 (Thbs1), a phospholipase A2 inhibitor, Cstb, Stfa1 and Serpinb6a, 6Anxa1, or a polynucleotide encoding said polypeptide. wherein the microglial cell or microglial-like cell is an activated microglial cell or microglial-like cell. In some embodiments, the microglial cell or microglial-like cell expresses one or more markers associated with an MG2 or MG3 microglial cell. In some embodiments, the microglial cell or microglial-like cell is characterized as having the following polypeptide expression profile: CD68-, SPP1-, P2Y12+, TMEM119+. In some embodiments, the microglial cell or microglial-like cell is characterized as having the following polypeptide expression profile: CD68-, SPP1-, P2Y12+, and/or TMEM119+. In some embodiments, the microglial cell or microglial-like cell expresses fibronectin and associated binding proteins; and/or one or more extracellular or intracellular peptidase inhibitors.

In some embodiments, the microglial cell or microglial-like cell is autologous or heterologous. In some embodiments, the heterologous microglial cell or microglial-like cell is derived from a mammal of the same or a different species than the subject. In some embodiments, a microglia cell or a microglial-like cell is differentiated from a stem cell. In some embodiments, a microglia cell or a microglial-like cell is differentiated from an embryonic stem cell. In some embodiments, a microglia cell or a microglial-like cell is differentiated from an induce pluripotent stem cell (iPSC). In some embodiments, the iPSC is a human iPSC. In some embodiments, the microglia cell or a microglial-like cell is differentiated from iPSCs as described in International Application Nos. PCT/US2018/019763 and PCT/US2016/039336, Abud et al., iPSC-Derived Human Microglia-like Cells to Study Neurological Diseases. Neuron 94, 278-293 (April 2017) and Muffat et al., Efficient derivation of microglia-like cells from human pluripotent stem cells. Nature Medicine, Vol. 22, No. 11 (November 2016), the entire contents of which are incorporated herein by reference.

In some embodiments, a treated microglia cell as described herein is a microglia cell that has been administered one or more peptidase inhibitors. In some embodiments, the treated microglia cell has been administered a serine peptidase inhibitor. In some embodiments, the serine peptidase inhibitor is Serpina3n, or an analog thereof. In some embodiments, Serpina3n is a polypeptide or fragment thereof having an amino acid sequence that is at least 85%, at least 90%, at least 95%, at least 98%, at least 99%, or 100% identical to a Serpina3n acid sequence associated with NCBI Reference Sequence: NP_033278.2. In some embodiments, Serpina3n is at least 85%, at least 90%, at least 95%, at least 98%, at least 99%, or 100% identical to the mTOR in Mus musculus.

In some embodiments, the treated microglia cell has been administered a cysteine peptidase inhibitor. In some embodiments, the cysteine peptidase inhibitor is E64, or an analog thereof, which has the chemical formula C₁₅H₂₇N₅O₅. E64 is a membrane-permeable irreversible inhibitor of a wide range of cysteine peptidases. In some embodiments, E64 has the following chemical structure:

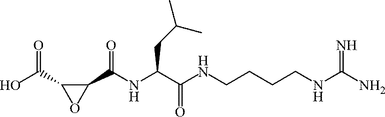 [SMILES: CC(C)C[C@H](NC(=O)[C@H]1O[C@@H]1C(=O)O)C(=O)NCCCCNC(=N)N]

In some embodiments, the treated microglia cell has been administered both a serine peptidase inhibitor and a cysteine peptidase inhibitor. In some embodiments, the treated microglia cell has been administered both E64 and Serpina3n, or analogs thereof. In some embodiments, the treated microglia cell has been administered with an inhibitor identified in FIGS. 14A-14C. In some embodiments, the treated microglia cell has been administered with a peptidase inhibitor that decreases expression of CD68. In some embodiments, the treated microglia cell has been administered with a peptidase inhibitor that decreases expression of SPP1. In some embodiments, the treated microglia cell has been administered with a peptidase inhibitor that decreases expression of CD68 and SPP1. In some embodiments, the treated microglia cell has been administered with a peptidase inhibitor that increases expression of P2Y12. In some embodiments, the treated microglia cell has been administered with a peptidase inhibitor that increases expression of TMEM119. In some embodiments, the treated microglia cell has been administered with a peptidase inhibitor that increases expression of P2Y12 and TMEM119. In some embodiments, the treated microglia cell has been administered with a peptidase inhibitor that decreases expression of CD68 and increases expression of P2Y12. In some embodiments, the treated microglia cell has been administered with a peptidase inhibitor that decreases expression of CD68 and increases expression of TMEM119. In some embodiments, the treated microglia cell has been administered with a peptidase inhibitor that decreases expression of CD68 and increases expression of P2Y12 and TMEM119. In some embodiments, the treated microglia cell has been administered with a peptidase inhibitor that decreases expression of SPP1 and increases expression of P2Y12. In some embodiments, the treated microglia cell has been administered with a peptidase inhibitor that decreases expression of SPP1 and increases expression of TMEM119. In some embodiments, the treated microglia cell has been administered with a peptidase inhibitor that decreases expression of SPP1 and increases expression of P2Y12 and TMEM119. In some embodiments, the treated microglia cell has been administered with a peptidase inhibitor that decreases expression of CD68 and SPP1 and increases expression of P2Y12. In some embodiments, the treated microglia cell has been administered with a peptidase inhibitor that decreases expression of CD68 and SPP1 and increases expression of TMEM119. In some embodiments, the treated microglia cell has been administered with a peptidase inhibitor that decreases expression of CD68 and SPP1 and increases expression of P2Y12 and TMEM119.

In some embodiments, treatment of a microglia cell with one or more peptidase inhibitors increases expression of Fnl and/or increases fibronectin production by the treated microglia cell. In some embodiments, treatment of a microglia cell with one or more peptidase inhibitors increases fibronectin production in a microglia cell of a subject relative to a reference, e.g., untreated cell. In some embodiments, treatment of a microglia cell with one or more peptidase inhibitors increases fibronectin production in a microglia cell by at least about 5%, at least about 10%, at least about 20%, at least about 30%, at least about 40%, at least about 50%, at least about 60%, at least about 70%, at least about 80%, at least about 90%, or at least about 100% relative to a reference, e.g., untreated cell.

In some embodiments, a treated microglia cell as described herein is a microglia cell that has been administered one or more phospholipase A2 inhibitors (e.g., Varespladib). In some embodiments, a treated microglia cell as described herein is a microglia cell that has been administered one or more peptidase inhibitors and one or more phospholipase A2 inhibitors (e.g., Varespladib). In some embodiments, the phospholipase A2 inhibitor is Varespladib (LY315920) or an analog thereof, which has the chemical formula C₂₁H₂₀N₂O₅. Varespladib is an inhibitor of the IIa, V, and X isoforms of secretory phospholipase A2 (sPLA2) and acts as an anti-inflammatory agent by disrupting the first step of the arachidonic acid pathway of inflammation. In some embodiments, Varespladib has the following chemical structure:

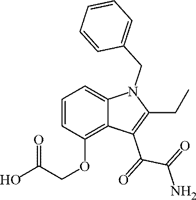 [SMILES: CCc1c(C(=O)C(N)=O)c2c(OCC(=O)O)cccc2n1Cc1ccccc1]

In some embodiments, treated microglia cells as described herein are used in methods for treating a subject with a neuroinflammatory disease characterized by the presence of reactive microglia by administering an effective amount of the treated microglia cell to the subject, wherein the microglia cell has been treated with one or more peptidase inhibitors. In some embodiments, the treated microglia cells as described herein are used in methods for treating a subject with a neurodegenerative disease characterized by the presence of reactive microglia by administering an effective amount of the treated microglia cell to the subject, wherein the microglia cell has been treated with one or more peptidase inhibitors. In some embodiments, the treated microglia cells as described herein are used in methods for treating neuronal injury by administering an effective amount of the treated microglia cell to the site of neuronal injury, wherein the microglia cell has been treated with one or more peptidase inhibitors. In some embodiments, treated microglia cells as described herein are used to treat neuronal injury in a subject. In some embodiments, treated microglia cells as described herein are used to treat neuronal injury in vitro. In some embodiments, the treated microglia cells as described herein are used in methods for promoting scar-free healing after neuronal injury by administering an effective amount of the treated microglia cell to the site of neuronal injury, wherein the microglia cell has been treated with one or more peptidase inhibitors. In some embodiments, treated microglia cells as described herein are used to promote scar-free healing after neuronal injury in a subject. In some embodiments, treated microglia cells as described herein are used to promote scar-free healing after neuronal injury in vitro. In some embodiments, the treated microglia cells as described herein are used in methods for promoting axon regeneration or regrowth by administering an effective amount of the treated microglia cell to the site in need of axon regeneration or regrowth, wherein the microglia cell has been treated with one or more peptidase inhibitors. In some embodiments, treated microglia cells as described herein are used to promote axon regeneration or regrowth in a subject. In some embodiments, treated microglia cells as described herein are used to promote axon regeneration or regrowth in vitro. In some embodiments, the treated microglia cells as described herein are used in methods for decreasing neuroinflammation by administering an effective amount of the treated microglia cell to the site of neuroinflammation, wherein the microglia cell has been treated with one or more peptidase inhibitors. In some embodiments, treated microglia cells as described herein are used to reduce neuroinflammation in a subject. In some embodiments, treated microglia cells as described herein are used to reduce neuroinflammation in vitro.

In some embodiments, the treated microglia cell described herein is administered to a subject via injection. In some embodiments, the treated microglia cell described herein is administered to a subject via transplantation. In some embodiments, injection and/or transplantation of the treated microglia cell described herein is administered to the spinal cord, brain, and/or to the site of neuronal injury.

While treatment methods may involve the administration of a treated microglia cell as provided herein, one skilled in the art will appreciate that more than one type of treated microglia cell may be used. For example, two or more microglia cells treated with one or more peptidase inhibitors may be used in the methods as provided herein. In some embodiments, a treated microglia cell (e.g., reactive microglia cells treated with one or more peptidase inhibitors) as described herein is used in combination with a neonatal microglia cell (e.g., neonatal microglia cell derived from a stem cell or iPSC). In some embodiments, one or more peptidase inhibitors (e.g., E64 and Serpina3n) are used in combination with a treated microglia cell (e.g., reactive microglia cells treated with one or more peptidase inhibitors). In some embodiments, one or more peptidase inhibitors (e.g., E64 and Serpina3n) are used in combination with a treated microglia cell (e.g., reactive microglia cells treated with one or more peptidase inhibitors) and a neonatal microglia cell (e.g., neonatal microglia cell derived from a stem cell or iPSC).

The treated microglia cell (e.g., reactive microglia cells treated with one or more peptidase inhibitors) may be incorporated into a pharmaceutical composition as provided herein for administration to a subject. In some embodiments, the treated microglia cell (e.g., reactive microglia cells treated with one or more peptidase inhibitors) is formulated for administration to a subject in need. In some embodiments, the treated microglia cell (e.g., reactive microglia cells treated with one or more peptidase inhibitors) is incorporated into a pharmaceutical composition for administration to a subject. In some embodiments, the treated microglia cell (e.g., reactive microglia cells treated with one or more peptidase inhibitors) is administered to a subject in need thereof to treat a disease or disorder (e.g., neuroinflammatory disease, neurodegenerative disease, and/or neuronal injury) and symptoms thereof. Pharmaceutical compositions as provided herein for administration to a subject may be formulated for one or more treated microglia cell (e.g., reactive microglia cells treated with one or more peptidase inhibitors).

The therapeutic methods of the disclosure in general comprise administration of an effective amount of the treated microglia cell (e.g., reactive microglia cells treated with one or more peptidase inhibitors) as described herein, such as a pharmaceutical composition thereof, to a subject (e.g., animal, human) in need thereof, including a mammal, particularly a human, to treat the disease or disorder or symptom thereof, under conditions such that the disease or disorder is treated to produce such effect. Such treatment will be suitably administered to subjects, particularly humans, suffering from, having, susceptible to, or at risk for a disease, disorder, or symptom thereof. Determination of those subjects in need of such treatment can be made by any objective or subjective determination by a diagnostic test or opinion of a subject or health care provider (e.g., genetic test, enzyme or protein marker, family history, and the like). The treated microglia cell (e.g., reactive microglia cells treated with one or more peptidase inhibitors) described herein may be also used in the treatment of any other disorders in which reactive microglia may be implicated.

### Neonatal Microglia Cells

The present disclosure features neonatal microglia cell (e.g., neonatal microglia cell derived from a stem cell or iPSC). Neonatal microglia cells as described herein may be used in any of the methods or combination therapy methods of the present invention. A neonatal microglia cell as described herein is a microglia cell that expresses neonatal markers.

In some embodiments, the neonatal microglia cell is characterized as having one or more of the following gene expression profiles: CD68-, SPP1-, P2Y12+, and/or TMEM119+. In some embodiments, the neonatal microglia cell is characterized as secreting fibronectin and associated binding proteins. In some embodiments, the neonatal microglia cell is characterized as expressing a number of extracellular and intracellular peptidase inhibitors. In some embodiments, the neonatal microglia cell is obtained from a neonatal subject or donor. In some embodiments, the neonatal microglia cell is differentiated in vitro. In some embodiments, the neonatal microglia cell is differentiated from a stem cell. In some embodiments, the stem cell is a human stem cell. In some embodiments, the neonatal microglia cell is differentiated from an induced pluripotent stem cell (iPSC). In some embodiments, the iPSC is a human iPSC. In some embodiments, a neonatal cell is differentiated from iPSCs as described in International Application Nos. PCT/US2018/019763 and PCT/US2016/039336, Abud et al., iPSC-Derived Human Microglia-like Cells to Study Neurological Diseases. Neuron 94, 278-293 (April 2017) and Muffat et al., Efficient derivation of microglia-like cells from human pluripotent stem cells. Nature Medicine, Vol. 22, No. 11 (November 2016), the entire contents of which are incorporated herein by reference.

In some embodiments, the neonatal microglia cells as described herein are used in methods for treating a subject with a neuroinflammatory disease characterized by the presence of reactive microglia by administering an effective amount of the neonatal microglia cell to the subject. In some embodiments, the neonatal microglia cells as described herein are used in methods for treating a subject with a neurodegenerative disease characterized by the presence of reactive microglia by administering an effective amount of the neonatal microglia cell to the subject. In some embodiments, the neonatal microglia cells as described herein are used in methods for treating neuronal injury by administering an effective amount of the neonatal microglia cell to the site of neuronal injury. In some embodiments, the neonatal microglia cells as described herein are used in methods for promoting scar-free healing after neuronal injury by administering an effective amount of the neonatal microglia cell to the site of neuronal injury. In some embodiments, the neonatal microglia cells as described herein are used in methods for promoting axon regeneration or regrowth by administering an effective amount of the neonatal microglia cell to the site in need of axon regeneration or regrowth. In some embodiments, the neonatal microglia cells as described herein are used in methods for decreasing neuroinflammation by administering an effective amount of the neonatal microglia cell to the site of neuroinflammation.

In some embodiments, the neonatal microglia cell described herein is administered to a subject via injection. In some embodiments, the neonatal microglia cell described herein is administered to a subject via transplantation. In some embodiments, injection and/or transplantation of the neonatal microglia cell described herein is administered to the spinal cord, brain, and/or to the site of neuronal injury.

In some embodiments, the neonatal microglia cell (e.g., neonatal microglia cell derived from a stem cell or iPSC) as described herein is used in combination with a treated microglia cell (e.g., reactive microglia cells treated with one or more peptidase inhibitors). In some embodiments, the one or more peptidase inhibitors (e.g., E64 and Serpina3n) are used in combination with a neonatal microglia cell (e.g., neonatal microglia cell derived from a stem cell or iPSC). In some embodiments, the one or more peptidase inhibitors (e.g., E64 and Serpina3n) are used in combination with a treated microglia cell (e.g., reactive microglia cells treated with one or more peptidase inhibitors) and a neonatal microglia cell (e.g., neonatal microglia cell derived from a stem cell or iPSC).

The neonatal microglia cell (e.g., neonatal microglia cell derived from a stem cell or iPSC) may be incorporated into a pharmaceutical composition as provided herein for administration to a subject. In some embodiments, the neonatal microglia cell (e.g., neonatal microglia cell derived from a stem cell or iPSC) is formulated for administration to a subject in need. In some embodiments, the neonatal microglia cell (e.g., neonatal microglia cell derived from a stem cell or iPSC) is incorporated into a pharmaceutical composition for administration to a subject. In some embodiments, the neonatal microglia cell (e.g., neonatal microglia cell derived from a stem cell or iPSC) is administered to a subject in need thereof to treat a disease or disorder (e.g., neuroinflammatory disease, neurodegenerative disease, and/or neuronal injury) and symptoms thereof. Pharmaceutical compositions as provided herein for administration to a subject may be formulated for one or more neonatal microglia cells (e.g., neonatal microglia cell derived from a stem cell or iPSC).

The therapeutic methods of the disclosure in general comprise administration of an effective amount of the neonatal microglia cell (e.g., neonatal microglia cell derived from a stem cell or iPSC) as described herein, such as a pharmaceutical composition thereof, to a subject (e.g., animal, human) in need thereof, including a mammal, particularly a human, to treat the disease or disorder or symptom thereof, under conditions such that the disease or disorder is treated to produce such effect. Such treatment will be suitably administered to subjects, particularly humans, suffering from, having, susceptible to, or at risk for a disease, disorder, or symptom thereof. Determination of those subjects in need of such treatment can be made by any objective or subjective determination by a diagnostic test or opinion of a subject or health care provider (e.g., genetic test, enzyme or protein marker, family history, and the like). The neonatal microglia cell (e.g., neonatal microglia cell derived from a stem cell or iPSC) described herein may be also used in the treatment of any other disorders in which reactive microglia may be implicated.

### Pharmaceutical Compositions

Compositions comprising one or more peptidase inhibitors (e.g., E64 and Serpina3n), treated microglia cells (e.g., reactive microglia cells treated with one or more peptidase inhibitors), and/or neonatal microglia cells (e.g., neonatal microglia cells derived from stem cells or iPSCs), as described herein, are provided. In some embodiments, pharmaceutical compositions of the present disclosure include one or more peptidase inhibitors (e.g., E64 and Serpina3n). In some embodiments, pharmaceutical compositions of the present disclosure include one or more phospholipase A2 inhibitors (e.g., Varespladib). In some embodiments, pharmaceutical compositions of the present disclosure include one or more peptidase inhibitors (e.g., E64 and Serpina3n) and one or more phospholipase A2 inhibitors (e.g., Varespladib). In some embodiments, the one or more phospholipase A2 inhibitors is Varespladib. In some embodiments, the one or more peptidase inhibitors is a serine peptidase inhibitor. In some embodiments, the serine peptidase inhibitor is Serpina3n, or an analog thereof. In some embodiments, the one or more peptidase inhibitors is a cysteine peptidase inhibitor. In some embodiments, the cysteine peptidase inhibitor is E64, or an analog thereof. In some embodiments, the peptidase inhibitors include both a serine peptidase inhibitor and a cysteine peptidase inhibitor. In some embodiments, the peptidase inhibitors include both E64 and Serpina3n, or analogs thereof. In some embodiments, the one or more peptidase inhibitor is an inhibitor identified in FIGS. 14A-14C. In some embodiments, the one or more peptidase inhibitors decreases the expression of CD68 and/or SPP1. In some embodiments, the one or more peptidase inhibitors increases the expression of P2Y12 and/or TMEM119. In some embodiments, the peptidase inhibitor decreases the expression of CD68 and/or SPP1 and increases the expression of P2Y12 and/or TMEM119.

In some embodiments, pharmaceutical compositions of the present disclosure include a treated microglia cell. In some embodiments, the treated microglia cell is a microglia cell that has been treated with one or more peptidase inhibitors. In some embodiments, the treated microglia cell is a reactive microglia cell. In some embodiments, the treated microglia cell is treated with one or more peptidase inhibitors in vitro. In some embodiments, the treated microglia cell is derived from a subject. In some embodiments, the treated microglia cell is derived from a donor. In some embodiments, the subject is an adult subject or donor. In some embodiments, the reactive microglia cell has one or more of the following gene expression profiles: CD68+, SPP1+, P2Y12-, and/or TMEM119-. In some embodiments, reactive microglia have persistent expression of Fnl and no significant induction of proteinase inhibitors.

In some embodiments, pharmaceutical compositions of the present disclosure include a neonatal microglia cell. In some embodiments, the neonatal microglia cell is characterized as having one or more of the following gene expression profiles: CD68-, SPP1-, P2Y12+, and/or TMEM119+. In some embodiments, the neonatal microglia cell is characterized as secreting fibronectin and associated binding proteins. In some embodiments, the neonatal microglia cell is characterized as expressing a number of extracellular and intracellular peptidase inhibitors. In some embodiments, the neonatal microglia cell is obtained from a neonatal subject or donor. In some embodiments, the neonatal microglia cell is differentiated in vitro. In some embodiments, the neonatal microglia cell is differentiated from a stem cell. In some embodiments, the stem cell is a human stem cell. In some embodiments, the neonatal microglia cell is differentiated from an induced pluripotent stem cell (iPSC). In some embodiments, the iPSC is a human iPSC.

In some embodiments, a pharmaceutical composition of the invention is administered to a subject identified as having a neuroinflammatory disease or disorder, neurodegenerative disease or disorder, and/or neuronal injury characterized by the presence of reactive microglia. The characterization of the presence of reactive microglia in a subject involves, for example, assessing the morphology and/or identifying the presence of increased levels of CD68 and/or SPP1 and/or decreased levels of P2Y12 and/or TMEM119 in microglia from a biological sample of the brain, spinal cord, and/or site of neuronal injury of the subject. In some embodiments, the neuroinflammatory disease is selected from Alzheimer’s disease, Parkinson’s disease, and/or multiple sclerosis, or a combination thereof. In some embodiments, the neurodegenerative disease is selected from Alzheimer’s disease, Parkinson’s disease, glaucoma, metachromatic leuokodystrophy, adrenoleukodystophy, lysosomal storage disorders, and/or amyotrophic lateral sclerosis, or a combination thereof. In some embodiments, the neuronal injury is selected from traumatic brain injury, spinal cord injury, spinal cord crush, and/or optic nerve injury, or a combination thereof.

Compositions and preparations (e.g., physiologically or pharmaceutically acceptable compositions) containing one or more peptidase inhibitors (e.g., E64 and Serpina3n), treated microglia cells (e.g., reactive microglia cells treated with one or more peptidase inhibitors), and/or neonatal microglia cells (e.g., neonatal microglia cells derived from stem cells or iPSCs) for administration include, without limitation, sterile aqueous or non-aqueous solutions, suspensions, and emulsions. Nonlimiting examples of non-aqueous solvents include propylene glycol, polyethylene glycol, vegetable oils, such as olive oil and canola oil, and injectable organic esters, such as ethyl oleate. Aqueous carriers include water, alcoholic/aqueous solutions, emulsions or suspensions, including saline and buffered media. Parenteral vehicles include, for example, sodium chloride solution, Ringer’s dextrose, dextrose and sodium chloride, lactated Ringer’s, or fixed oils. Intravenous vehicles include, for example, fluid and nutrient replenishers, electrolyte replenishers (such as those based on Ringer’s dextrose), and the like. Preservatives and other additives may also be present in such compositions and preparations, such as, for example, antimicrobials, antioxidants, chelating agents, colorants, stabilizers, inert gases and the like.

Some of the compositions may potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

Provided herein are pharmaceutical compositions which include an effective amount of one or more peptidase inhibitors (e.g., E64 and Serpina3n), treated microglia cells (e.g., reactive microglia cells treated with one or more peptidase inhibitors), and/or neonatal microglia cells (e.g., neonatal microglia cells derived from stem cells or iPSCs), alone, or in combination with a pharmaceutically acceptable carrier or excipient. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid or aqueous solution, suspension, emulsion, dispersion, tablet, pill, capsule, powder, or sustained release formulation. A liquid or aqueous composition can be lyophilized and reconstituted with a solution or buffer prior to use. The composition can be formulated as a suppository, with traditional binders and carriers such as triglycerides. Oral formulations can include standard carriers, such as pharmaceutical grades of mannitol, lactose, starch, magnesium stearate, sodium saccharine, cellulose, and magnesium carbonate. Any of the commonly known pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used in the compositions and administration methods as described are normal saline and sesame oil.

The compositions can also contain other medicinal agents, pharmaceutical agents, adjuvants, carriers, and auxiliary substances such as wetting or emulsifying agents, and pH buffering agents. Standard texts, such as Remington: The Science and Practice of Pharmacy, 17th edition, Mack Publishing Company, incorporated herein by reference, can be consulted to prepare suitable compositions and formulations for administration, without undue experimentation. Suitable dosages can also be based upon the text and documents cited herein. A determination of the appropriate dosages is within the skill of one in the art given the parameters herein.

A therapeutically effective amount of a pharmaceutical composition can be administered in one or more doses. In terms of treatment, an effective amount is an amount that is sufficient to palliate, ameliorate, stabilize, reverse or slow the progression of a neuroinflammatory disease or disorder, neurodegenerative disease or disorder, and/or neuronal injury characterized by the presence of reactive microglia. A therapeutically effective amount can be provided in one or a series of administrations. The effective amount is generally determined by the physician on a case-by-case basis and is within the skill of one in the art.

As a rule, the dosage for in vivo therapeutics or diagnostics will vary. Several factors are typically taken into account when determining an appropriate dosage. These factors include age, sex and weight of the patient, the condition being treated, the severity of the condition and the form of the pharmaceutical composition being administered. In various embodiments, a dosage ranging from about 0.5 to about 100 mg/kg of body weight is useful; or any dosage range in which the low end of the range is any amount between 0.1 mg/kg/day and 90 mg/kg/day and the upper end of the range is any amount between 1 mg/kg/day and 100 mg/kg/day (e.g., 0.5 mg/kg/day and 5 mg/kg/day, 25 mg/kg/day and 75 mg/kg/day).

Administrations can be conducted infrequently, or on a regular weekly basis until a desired, measurable parameter is detected, such as diminution of disease symptoms. Administration can then be diminished, such as to a biweekly or monthly basis, as appropriate.

Compositions of the present invention comprising one or more peptidase inhibitors (e.g., E64 and Serpina3n), treated microglia cells (e.g., reactive microglia cells treated with one or more peptidase inhibitors), and/or neonatal microglia cells (e.g., neonatal microglia cells derived from stem cells or iPSCs) are administered by a mode appropriate for the form of composition. Available routes of administration include subcutaneous, intramuscular, intraperitoneal, intradermal, oral, intranasal, intrapulmonary (i.e., by aerosol), intravenously, intramuscularly, subcutaneously, intracavity, intrathecally or transdermally.

Compositions for oral, intranasal, or topical administration can be supplied in solid, semisolid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, compositions are preferably supplied in unit dosage form suitable for administration of a precise amount. Also contemplated by this invention are slow release or sustained release forms, whereby a relatively consistent level of the active compound are provided over an extended period.

In some embodiments, the pharmaceutical compositions described herein are administered via injection. In some embodiments, the pharmaceutical compositions described herein are administered via transplantation. In some embodiments, injection and/or transplantation of the pharmaceutical compositions described herein are administered to the spinal cord, brain, and/or to the site of neuronal injury.

### Methods of Treatment, Methods of Use, Administration and Delivery

Methods of treating a disease, or symptoms thereof, are provided. The present disclosure features methods that are useful for the treatment of neuroinflammatory diseases or disorders. In some embodiments, the neuroinflammatory disease includes but is not limited to Alzheimer’s disease, Parkinson’s disease, and/or multiple sclerosis, or a combination thereof. The present disclosure also features methods that are useful for the treatment of neurodegenerative diseases or disorders. In some embodiments, the neurodegenerative disease includes but is not limited to Alzheimer’s disease, Parkinson’s disease, glaucoma, metachromatic leuokodystrophy, adrenoleukodystophy, lysosomal storage disorders, and/or amyotrophic lateral sclerosis, or a combination thereof. In some embodiments, the present disclosure features methods that are useful for the treatment of neuronal injury. In some embodiments, the neuronal injury includes but is not limited to traumatic brain injury, spinal cord injury, spinal cord crush, and/or optic nerve injury, or a combination thereof. The methods of treating a subject having a neuroinflammatory disease or disorder, neurodegenerative disease or disorder, and/or neuronal injury are characterized by the presence of reactive microglia. The characterization of the presence of reactive microglia in a subject involves, for example, assessing the morphology and/or identifying the presence of increased levels of CD68 and/or SPP1 and/or decreased levels of P2Y12 and/or TMEM119 in microglia from a biological sample of the brain, spinal cord, and/or site of neuronal injury of the subject. In some embodiments, the reactive microglia cell has one or more of the following gene expression profiles: CD68+, SPP1+, P2Y12-, and/or TMEM119-. The present disclosure features methods that are useful for promoting scar-free healing after neuronal injury. The present disclosure also features methods that are useful for promoting axon regeneration or regrowth. The present disclosure also features methods that are useful for decreasing neuroinflammation. The present disclosure further features methods that are useful for re-establishing microglia homeostasis.

The methods of the present disclosure include administering a therapeutically effective amount of one or more peptidase inhibitors (e.g., E64 and Serpina3n), treated microglia cells (e.g., reactive microglia cells treated with one or more peptidase inhibitors), and/or neonatal microglia cells (e.g., neonatal microglia cells derived from stem cells or iPSCs) as provided herein, or pharmaceutical compositions thereof, to a subject (e.g., a mammal, such as a human). The methods of the present disclosure include administering a therapeutically effective amount of one or more phospholipase A2 inhibitors (e.g., Varespladib), treated microglia cells (e.g., reactive microglia cells treated with one or more phospholipase A2 inhibitors), and/or neonatal microglia cells (e.g., neonatal microglia cells derived from stem cells or iPSCs) as provided herein, or pharmaceutical compositions thereof, to a subject (e.g., a mammal, such as a human). In some embodiments, the one or more phospholipase A2 inhibitors is Varespladib. In some embodiments, the subject is a mammalian subject. In some embodiments, the subject is a human subject. In some embodiments, the subject is an adult subject.

In some embodiments, the methods of the present disclosure include treating a subject with a neuroinflammatory disease characterized by the presence of reactive microglia by administering an effective amount of one or more peptidase inhibitors to a reactive microglia cell in the subject. In some embodiments, the methods of the present disclosure include treating a subject with a neurodegenerative disease characterized by the presence of reactive microglia by administering an effective amount of one or more peptidase inhibitors to a reactive microglia cell in the subject. In some embodiments, the methods of the present disclosure include treating neuronal injury in a subject by administering an effective amount of one or more peptidase inhibitors to a reactive microglia cell in the subject at the site of neuronal injury. In some embodiments, the methods of the present disclosure include promoting scar-free healing after neuronal injury in a subject by administering an effective amount of one or more peptidase inhibitors to a reactive microglia cell in the subject at the site of neuronal injury. In some embodiments, the methods of the present disclosure include promoting axon regeneration or regrowth in a subject by administering an effective amount of one or more peptidase inhibitors to a reactive microglia cell in the subject at the site for axon regeneration or regrowth. In some embodiments, the methods of the present disclosure include decreasing neuroinflammation in a subject by administering an effective amount of one or more peptidase inhibitors to a reactive microglia cell in the subject at the site of neuroinflammation. In some embodiments, the methods of the present disclosure include re-establishing microglia homeostasis by treating a reactive microglia cell with one or more peptidase inhibitors. In some embodiments, the method of re-establishing microglia homeostasis is conducted in vitro.

In some embodiments, the one or more peptidase inhibitors is a serine peptidase inhibitor. In some embodiments, the serine peptidase inhibitor is Serpina3n, or an analog thereof. In some embodiments, the one or more peptidase inhibitors is a cysteine peptidase inhibitor. In some embodiments, the cysteine peptidase inhibitor is E64, or an analog thereof. In some embodiments, the peptidase inhibitors include both a serine peptidase inhibitor and a cysteine peptidase inhibitor. In some embodiments, the peptidase inhibitors include both E64 and Serpina3n, or analogs thereof. In some embodiments, the one or more peptidase inhibitor is an inhibitor identified in FIGS. 14A-14C. In some embodiments, the one or more peptidase inhibitors decreases the expression of CD68 and/or SPP1. In some embodiments, the one or more peptidase inhibitors increases the expression of P2Y12 and/or TMEM119. In some embodiments, the peptidase inhibitor decreases the expression of CD68 and/or SPP1 and increases the expression of P2Y12 and/or TMEM119.

In some embodiments, the methods of the present disclosure include treating a subject with a neuroinflammatory disease characterized by the presence of reactive microglia by administering to the subject an effective amount of a treated microglia cell, wherein the microglia cell has been treated with one or more peptidase inhibitors. In some embodiments, the methods of the present disclosure include treating a subject with a neurodegenerative disease characterized by the presence of reactive microglia by administering to the subject an effective amount of a treated microglia cell, wherein the microglia cell has been treated with one or more peptidase inhibitors. In some embodiments, the methods of the present disclosure include treating neuronal injury in a subject by administering to the subject at the site of neuronal injury an effective amount of a treated microglia cell, wherein the microglia cell has been treated with one or more peptidase inhibitors. In some embodiments, the methods of the present disclosure include promoting scar-free healing after neuronal injury in a subject by administering to the subject at the site of neuronal injury an effective amount of a treated microglia cell, wherein the microglia cell has been treated with one or more peptidase inhibitors. In some embodiments, the methods of the present disclosure include promoting axon regeneration or regrowth in a subject by administering to the subject at the site for axon regeneration or regrowth an effective amount of a treated microglia cell, wherein the microglia cell has been treated with one or more peptidase inhibitors. In some embodiments, the methods of the present disclosure include reducing neuroinflammation in a subject by administering to the subject at the site of neuroinflammation an effective amount of a treated microglia cell, wherein the microglia cell has been treated with one or more peptidase inhibitors.

In some embodiments, the treated microglia cell is an microglia cell that has been treated with one or more peptidase inhibitors. In some embodiments, the treated microglia cell is a reactive microglia cell that has been treated with one or more peptidase inhibitors. In some embodiments, the treated microglia cell is treated with one or more peptidase inhibitors in vitro. In some embodiments, the treated microglia cell is derived from a subject. In some embodiments, the treated microglia cell is derived from a donor. In some embodiments, the reactive microglia cell has one or more of the following gene expression profiles: CD68+, SPP1+, P2Y12-, and/or TMEM119-. In some embodiments, reactive microglia have persistent expression of Fnl and no significant induction of proteinase inhibitors.

In some embodiments, the methods of the present disclosure include treating a subject with a neuroinflammatory disease characterized by the presence of reactive microglia by administering to the subject an effective amount of a neonatal microglia cell. In some embodiments, the methods of the present disclosure include treating a subject with a neurodegenerative disease characterized by the presence of reactive microglia by administering to the subject an effective amount of a neonatal microglia cell. In some embodiments, the methods of the present disclosure include treating neuronal injury in a subject by administering to the subject at the site of neuronal injury an effective amount of a neonatal microglia cell. In some embodiments, the methods of the present disclosure include promoting scar-free healing after neuronal injury in a subject by administering to the subject at the site of neuronal injury an effective amount of a neonatal microglia cell. In some embodiments, the methods of the present disclosure include promoting axon regeneration or regrowth in a subject by administering to the subject at the site for axon regeneration or regrowth an effective amount of a neonatal microglia cell. In some embodiments, the methods of the present disclosure include decreasing neuroinflammation in a subject by administering to the subject at the site of neuroinflammation an effective amount of a neonatal microglia cell.

In some embodiments, the neonatal microglia cell is characterized as having one or more of the following gene expression profiles: CD68-, SPP1-, P2Y12+, and/or TMEM119+. In some embodiments, the neonatal microglia cell is characterized as secreting fibronectin and associated binding proteins. In some embodiments, the neonatal microglia cell is characterized as expressing a number of extracellular and intracellular peptidase inhibitors. In some embodiments, the neonatal microglia cell is obtained from a neonatal subject or donor. In some embodiments, the neonatal microglia cell is differentiated in vitro. In some embodiments, the neonatal microglia cell is differentiated from a stem cell. In some embodiments, the stem cell is a human stem cell. In some embodiments, the neonatal microglia cell is differentiated from an induced pluripotent stem cell (iPSC). In some embodiments, the iPSC is a human iPSC.

The treatment methods provided herein are suitably administered to subjects, particularly humans, suffering from, are susceptible to, or at risk of having a disease, or symptoms thereof. Identifying a subject in need of such treatment can be based on the judgment of the subject or of a health care professional and can be subjective (e.g., opinion) or objective (e.g., measurable by a test or diagnostic method). Briefly, the determination of those subjects who are in need of treatment or who are “at risk” or “susceptible” can be made by any objective or subjective determination by a diagnostic test (e.g., blood sample, biopsy, genetic test, enzyme or protein marker assay), marker analysis, family history, and the like, including an opinion of the subject or a health care provider. In some embodiments, the subject in need of treatment can be identified by, for example, measuring gene expression levels (e.g. CD68, SPP1, P2Y12, TMEM119) or from analyzing the morphology of microglia collected from a patient (e.g., tissue sample). The methods and compositions as described herein may also be used in the treatment of any other diseases or disorders in which reactive microglia may be implicated. A subject undergoing treatment can be a non-human mammal, such as a veterinary subject, or a human subject (also referred to as a “patient”).

The methods of the present disclosure include methods of monitoring the progress of disease (e.g., neuroinflammatory disease, neurodegenerative disease, and/or neuronal injury) characterized by the presence of reactive microglia. The methods of the present disclosure also include methods of monitoring treatment of the disease (e.g., neuroinflammatory disease, neurodegenerative disease, and/or neuronal injury). These methods include a diagnostic measurement (e.g., biopsy, CT scan, screening assay or detection assay) in a subject suffering from or susceptible to disease or symptoms thereof, in which the subject has been administered an amount (e.g., a therapeutic amount) of a pharmaceutical composition as described herein, sufficient to treat the disease or symptoms thereof. The diagnostic measurement in the method can be compared to samples from healthy, normal controls; in a pre-disease sample of the subject; or in other afflicted/diseased patients to establish the treated subject’s disease status. For monitoring, a second diagnostic measurement may be obtained from the subject at a time point later than the determination of the first diagnostic measurement, and the two measurements can be compared to monitor the course of disease or the efficacy of the therapy/treatment. In certain embodiments, a pre-treatment measurement in the subject (e.g., in a sample or biopsy obtained from the subject or CT scan) is determined prior to beginning treatment as described; this measurement can then be compared to a measurement in the subject after the treatment commences and/or during the course of treatment to determine the efficacy of (monitor the efficacy of) the disease treatment. In some embodiments, the diagnostic measurement is the gene expression profile from microglia obtained from a biological sample of the subject.

The one or more peptidase inhibitors (e.g., E64 and Serpina3n), treated microglia cells (e.g., reactive microglia cells treated with one or more peptidase inhibitors), and/or neonatal microglia cells (e.g., neonatal microglia cells derived from stem cells or iPSCs), or pharmaceutical compositions thereof, as provided herein can be administered to a subject by any of the routes normally used for introducing a compound into a subject. Routes and methods of administration include, without limitation, intradermal, intramuscular, intraperitoneal, intrathecal, parenteral, such as intravenous (IV) or subcutaneous (SC), vaginal, rectal, intranasal, inhalation, intraocular, intracranial, or oral. Parenteral administration, such as subcutaneous, intravenous or intramuscular administration, is generally achieved by injection (immunization). Injectables can be prepared in conventional forms and formulations, either as liquid solutions or suspensions, solid forms (e.g., lyophilized forms) suitable for solution or suspension in liquid prior to injection, or as emulsions. Injection solutions and suspensions can be prepared from sterile powders, granules, and tablets. The one or more peptidase inhibitors (e.g., E64 and Serpina3n), treated microglia cells (e.g., reactive microglia cells treated with one or more peptidase inhibitors), and/or neonatal microglia cells (e.g., neonatal microglia cells derived from stem cells or iPSCs) or pharmaceutical compositions thereof, can be administered as described herein in any suitable manner, such as with pharmaceutically acceptable carriers, diluents, or excipients as described supra. Pharmaceutically acceptable carriers are determined in part by the particular immunogen or composition being administered, as well as by the particular method used to administer the composition. Accordingly, pharmaceutical composition comprising one or more peptidase inhibitors (e.g., E64 and Serpina3n), treated microglia cells (e.g., reactive microglia cells treated with one or more peptidase inhibitors), and/or neonatal microglia cells (e.g., neonatal microglia cells derived from stem cells or iPSCs) can be prepared using a wide variety of suitable and physiologically and pharmaceutically acceptable formulations for use in the methods of the present disclosure.

The methods herein include administering to the subject (including a subject identified as in need of such treatment) an effective amount of microglia cells (e.g., treated microglia cells, neonatal microglia cells) described herein, or a pharmaceutical composition comprising such cells as described herein to produce such effect. Such treatment will be suitably administered to a subject, particularly a human, suffering from, having, susceptible to, or at risk for, a disease, or a symptom thereof (e.g., neuroinflammatory disease, neurodegenerative disease, and/or neuronal injury). In some embodiments, the methods herein include administering to the subject (including a subject identified as in need of such treatment) an effective amount of a compound described herein, or a composition described herein to produce such effect.

In some embodiments, the microglia cell (e.g., treated microglia cell, neonatal microglia cell), or pharmaceutical composition thereof, is administered to a subject in a targeted manner. For example, in some embodiments, a composition comprising a microglia cell (e.g., treated microglia cell, neonatal microglia cell) is administered directly to a subject’s spinal cord and/or brain. In some embodiments, the composition is delivered directly to the brain via intracerebroventricular administration. In some embodiments, the composition is delivered in this manner to the lateral ventricles of the subject’s brain.

Alternatively, the composition may be delivered systemically, such as by intravenous administration. Cells administered in such a manner must traverse the blood brain barrier prior to engrafting in the subject’s brain. Other modes of administration (parenteral, mucosal, implant, intraperitoneal, intradermal, transdermal, intramuscular, intracerebroventricular injection, intravenous including infusion and/or bolus injection, and subcutaneous) are generally known in the art. In some embodiments, microglia cells (e.g., treated microglia cells, neonatal microglia cells) are administered in a medium suitable for injection, such as phosphate buffered saline, into a subject. In some embodiments, intracerebroventricular administration may be advantageous to routes that require crossing the blood brain barrier.

In some embodiments, the transplanted cells are meant to replace endogenous cells (i.e., reactive microglial cells); therefore, methods of treating a subject having, susceptible to, or at risk of developing a disease or disorder further comprise administering to a subject an agent for ablating endogenous reactive microglia cells prior to administering a treated microglia cell and/or neonatal microglia cell. In some embodiments, the agent is an alkylating agent. In some embodiments, the alkylating agent is busulfan. In some embodiments, nanoparticle delivery of alkylating agents may be effective in creating a suitable environment for engraftment of transplanted cells, as described in International Application No. PCT/US2017/056774, the contents of which are incorporated herein by reference in their entirety.

Administration of the one or more peptidase inhibitors (e.g., E64 and Serpina3n), treated microglia cells (e.g., reactive microglia cells treated with one or more peptidase inhibitors), and/or neonatal microglia cells (e.g., neonatal microglia cells derived from stem cells or iPSCs), or pharmaceutical compositions thereof, can be accomplished by single or multiple doses. The dose administered to a subject should be sufficient to induce a beneficial therapeutic response in a subject over time, such as to inhibit, block, reduce, ameliorate, protect against, or prevent disease (e.g., neuroinflammatory disease, neurodegenerative disease, and/or neuronal injury). The dose required will vary from subject to subject depending on the species, age, weight and general condition of the subject, by the severity of the disease or disorder being treated, by the particular composition being used and by the mode of administration. An appropriate dose can be determined by a person skilled in the art, such as a clinician or medical practitioner, using only routine experimentation. One of skill in the art is capable of determining therapeutically effective amounts of the compositions provided herein, that provide a therapeutic effect or protection against diseases (e.g., neuroinflammatory disease, neurodegenerative disease, and/or neuronal injury) suitable for administering to a subject in need of treatment or protection.

### Combination Therapies

The one or more peptidase inhibitors (e.g., E64 and Serpina3n), treated microglia cells (e.g., reactive microglia cells treated with one or more peptidase inhibitors), and/or neonatal microglia cells (e.g., neonatal microglia cells derived from stem cells or iPSCs) as described herein, or pharmaceutical compositions thereof, can be administered alone or in combination with each other to treat a subject having a neuroinflammatory disease or disorder, neurodegenerative disease or disorder, and/or neuronal injury characterized by the presence of reactive microglia. The characterization of the presence of reactive microglia in a subject involves, for example, assessing the morphology and/or identifying the presence of increased levels of CD68 and/or SPP1 and/or decreased levels of P2Y12 and/or TMEM119 in microglia from a biological sample of the brain, spinal cord, and/or site of neuronal injury of the subject. In some embodiments, the neuroinflammatory disease includes but is not limited to Alzheimer’s disease, Parkinson’s disease, and/or multiple sclerosis, or a combination thereof. In some embodiments, the neurodegenerative disease includes but is not limited to Alzheimer’s disease, Parkinson’s disease, glaucoma, metachromatic leuokodystrophy, adrenoleukodystophy, lysosomal storage disorders, and/or amyotrophic lateral sclerosis, or a combination thereof. In some embodiments, the neuronal injury includes but is not limited to traumatic brain injury, spinal cord injury, spinal cord crush, and/or optic nerve injury, or a combination thereof.

In some embodiments, the one or more peptidase inhibitors (e.g., E64 and Serpina3n) are used in combination with a treated microglia cell (e.g., reactive microglia cells treated with one or more peptidase inhibitors). In some embodiments, the one or more peptidase inhibitors (e.g., E64 and Serpina3n) are used in combination with a neonatal microglia cell (e.g., neonatal microglia cell derived from a stem cell or iPSC). In some embodiments, the one or more peptidase inhibitors (e.g., E64 and Serpina3n) are used in combination with a treated microglia cell (e.g., reactive microglia cells treated with one or more peptidase inhibitors) and a neonatal microglia cell (e.g., neonatal microglia cell derived from a stem cell or iPSC). In some embodiments, the treated microglia cell (e.g., reactive microglia cells treated with one or more peptidase inhibitors) is used in combination with a neonatal microglia cell (e.g., neonatal microglia cell derived from a stem cell or iPSC).

In some embodiments, one or more peptidase inhibitors (e.g., E64 and Serpina3n) are used in combination with one or more phospholipase A2 inhibitors (e.g., Varespladib). In some embodiments, the one or more peptidase inhibitors (e.g., E64 and Serpina3n) are used in combination with a treated microglia cell (e.g., reactive microglia cells treated with one or more phospholipase A2 inhibitors). In some embodiments, the one or more peptidase inhibitors (e.g., E64 and Serpina3n) are used in combination with a treated microglia cell (e.g., reactive microglia cells treated with one or more phospholipase A2 inhibitors) and a neonatal microglia cell (e.g., neonatal microglia cell derived from a stem cell or iPSC).

In some embodiments, the one or more phospholipase A2 inhibitors (e.g., E64 and Serpina3n) are used in combination with a treated microglia cell (e.g., reactive microglia cells treated with one or more peptidase inhibitors). In some embodiments, the one or more phospholipase A2 inhibitors (e.g., E64 and Serpina3n) are used in combination with a treated microglia cell (e.g., reactive microglia cells treated with one or more phospholipase A2 inhibitors). In some embodiments, the one or more phospholipase A2 inhibitors (e.g., E64 and Serpina3n) are used in combination with a neonatal microglia cell (e.g., neonatal microglia cell derived from a stem cell or iPSC). In some embodiments, the one or more phospholipase A2 inhibitors (e.g., E64 and Serpina3n) are used in combination with a treated microglia cell (e.g., reactive microglia cells treated with one or more phospholipase A2 inhibitors) and a neonatal microglia cell (e.g., neonatal microglia cell derived from a stem cell or iPSC). In some embodiments, the one or more phospholipase A2 inhibitors (e.g., E64 and Serpina3n) are used in combination with a treated microglia cell (e.g., reactive microglia cells treated with one or more peptidase inhibitors) and a neonatal microglia cell (e.g., neonatal microglia cell derived from a stem cell or iPSC). In some embodiments, the one or more phospholipase A2 inhibitors is Varespladib.

In some embodiments, one or more peptidase inhibitors are used in the combination therapy methods of the present invention. In some embodiments, the one or more peptidase inhibitors is a serine peptidase inhibitor. In some embodiments, the serine peptidase inhibitor is Serpina3n, or an analog thereof. In some embodiments, the one or more peptidase inhibitors is a cysteine peptidase inhibitor. In some embodiments, the cysteine peptidase inhibitor is E64, or an analog thereof. In some embodiments, the peptidase inhibitors include both a serine peptidase inhibitor and a cysteine peptidase inhibitor. In some embodiments, the peptidase inhibitors include both E64 and Serpina3n, or analogs thereof. In some embodiments, the one or more peptidase inhibitor is an inhibitor identified in FIGS. 14A-14C. In some embodiments, the one or more peptidase inhibitors decreases the expression of CD68 and/or SPP1. In some embodiments, the one or more peptidase inhibitors increases the expression of P2Y12 and/or TMEM119. In some embodiments, the peptidase inhibitor decreases the expression of CD68 and/or SPP1 and increases the expression of P2Y12 and/or TMEM119.

In some embodiments, a treated microglia cell is used in the combination therapy methods of the present invention. In some embodiments, the treated microglia cell is a microglia cell that has been treated with one or more peptidase inhibitors. In some embodiments, the treated microglia cell is a reactive microglia cell that has been treated with one or more peptidase inhibitors. In some embodiments, the treated microglia cell is treated with one or more peptidase inhibitors in vitro. In some embodiments, the treated microglia cell is derived from a subject. In some embodiments, the treated microglia cell is derived from a donor. In some embodiments, the subject is an adult subject or donor. In some embodiments, the treated reactive microglia cell has one or more of the following gene expression profiles: CD68+, SPP1+, P2Y12-, and/or TMEM119-. In some embodiments, the treated reactive microglia cell has persistent expression of Fnl and no significant induction of proteinase inhibitors.

In some embodiments, a neonatal microglia cell is used in the combination therapy methods of the present invention. In some embodiments, the neonatal microglia cell is characterized as having one or more of the following gene expression profiles: CD68-, SPP1-, P2Y12+, and/or TMEM119+. In some embodiments, the neonatal microglia cell is characterized as secreting fibronectin and associated binding proteins. In some embodiments, the neonatal microglia cell is characterized as expressing a number of extracellular and intracellular peptidase inhibitors. In some embodiments, the neonatal microglia cell is obtained from a neonatal subject or donor. In some embodiments, the neonatal microglia cell is differentiated in vitro. In some embodiments, the neonatal microglia cell is differentiated from a stem cell. In some embodiments, the stem cell is a human stem cell. In some embodiments, the neonatal microglia cell is differentiated from an induced pluripotent stem cell (iPSC). In some embodiments, the iPSC is a human iPSC.

### Kits

The invention provides kits for the treatment of a subject having a neuroinflammatory disease or disorder, neurodegenerative disease or disorder, and/or neuronal injury characterized by the presence of reactive microglia. In some embodiments, the neuroinflammatory disease includes but is not limited to Alzheimer’s disease, Parkinson’s disease, and/or multiple sclerosis, or a combination thereof. In some embodiments, the neurodegenerative disease includes but is not limited to Alzheimer’s disease, Parkinson’s disease, glaucoma, metachromatic leuokodystrophy, adrenoleukodystophy, lysosomal storage disorders, and/or amyotrophic lateral sclerosis, or a combination thereof. In some embodiments, the neuronal injury includes but is not limited to traumatic brain injury, spinal cord injury, spinal cord crush, and/or optic nerve injury, or a combination thereof.

In some embodiments, the kit includes one or more peptidase inhibitors as described herein. In some embodiments, the kit includes one or more phospholipase A2 inhibitors as described herein. In some embodiments, the kit includes a microglia cell as described herein, wherein the microglia cell has been treated with one or more peptidase inhibitors and/or phospholipase A2 inhibitors. In some embodiments, the kit includes a neonatal microglia cell as described herein. In some embodiments, the kit includes a combination of agents selected from two or more of: 1) one or more peptidase inhibitors; 2) one or more phospholipase A2 inhibitors; 3) a microglia cell, wherein the microglia cell has been treated with one or more peptidase inhibitors and/or phospholipase A2 inhibitors; and/or 4) a neonatal microglia cell. In some embodiments, the kit includes one or more peptidase inhibitors and/or phospholipase A2 inhibitors and a microglia cell, wherein the microglia cell has been treated with one or more peptidase inhibitors and/or phospholipase A2 inhibitors. In some embodiments, the kit includes one or more peptidase inhibitors and/or phospholipase A2 inhibitors and a neonatal microglia cell. In some embodiments, the kit includes a microglia cell, wherein the microglia cell has been treated with one or more peptidase inhibitors and/or phospholipase A2 inhibitors and a neonatal microglia cell. In some embodiments, the kit includes a pharmaceutical composition comprising a combination of agents selected from two or more of: 1) one or more peptidase inhibitors; 2) one or more phospholipase A2 inhibitors; 3) a microglia cell, wherein the microglia cell has been treated with one or more peptidase inhibitors; and/or 4) a neonatal microglia cell.

The kits of the present disclosure may also comprise one or more of the compositions or reagents described herein in any number of separate containers, packets, tubes (e.g., <0.2 ml, 0.2 ml, 0.6 ml, 1.5 ml, 5.0 ml, >5.0 ml), vials, microtiter plates (e.g., <96-well, 96-well, 384-well, 1536-well, >1536- well), ArrayTape, and the like, or the compositions or reagents described herein may be combined in various combinations in such containers. In yet other embodiments, the kit comprises a sterile container which contains the one or more compositions or reagents described herein; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids.

The components may, for example, be dried (e.g., dry residue), lyophilized (e.g., dry cake) or in a stable buffer (e.g., chemically stabilized, thermally stabilized). Dry components may, for example, be prepared by lyophilization, vacuum and centrifugal assisted drying and/or ambient drying.

If desired an agent of the invention is provided together with instructions for administering the agent to a subject. The instructions will generally include information about the use of the composition for the treatment of the disease or disorder (e.g., neuroinflammatory disease, neurodegenerative disease, and/or neuronal injury). In other embodiments, the instructions include at least one of the following: description of the therapeutic agent; dosage schedule and administration for treatment of a disease or symptoms thereof (e.g., neuroinflammatory disease, neurodegenerative disease, and/or neuronal injury); precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of an artisan of ordinary skill in the art. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Scar-Free Healing and Axon Regrowth After Spinal Cord Injury in Neonatal Mice is Orchestrated by Microglia

Spinal cord injury disrupts axonal connections between the brain and the spinal cord below the lesion. In fish and amphibians, meningeal cells and glia form a permissive bridge, allowing injured axons to regenerate across the lesion for functional restoration (Mokalled, M. H. et al. Injury-induced ctgfa directs glial bridging and spinal cord regeneration in zebrafish. Science 354, 630-634, doi:10.1126/science.aaf2679 (2016); Tsarouchas, T. M. et al. Dynamic control of proinflammatory cytokines Il-1beta and Tnf-alpha by macrophages in zebrafish spinal cord regeneration. Nature communications 9, 4670, doi:10.1038/s41467-018-07036-w (2018); Zukor, K. A., et al., Meningeal cells and glia establish a permissive environment for axon regeneration after spinal cord injury in newts. Neural development 6, 1, doi:10.1186/1749-8104-6-1 (2011); Bellver-Landete, V. et al. Microglia are an essential component of the neuroprotective scar that forms after spinal cord injury. Nature communications 10, 518, doi:10.1038/s41467-019-08446-0 (2019)). However, this is not the case in adult mammals. Instead, a spinal cord injury triggers the formation of a scar, consisting of multiple cell types such as reactive astrocytes, fibroblasts, and microglia/macrophages, resulting in no spontaneous axon regeneration (O′Shea, T.M. et al., Cell biology of spinal cord injury and repair. The Journal of clinical investigation 127, 3259-3270, doi:10.1172/JCI90608 (2017); Hilton, B. J. & Bradke, F. Can injured adult CNS axons regenerate by recapitulating development? Development 144, 3417-3429, doi:10.1242/dev.148312 (2017); Tran, A. P., et al., The Biology of Regeneration Failure and Success After Spinal Cord Injury. Physiological reviews 98, 881-917, doi:10.1152/physrev.00017.2017 (2018); Courtine, G. & Sofroniew, M. V. Spinal cord repair: advances in biology and technology. Nature medicine 25, 898-908, doi:10.1038/s41591-019-0475-6 (2019)).

Despite studies about these different cell types and their associated molecules, how the spinal cord responds to an injury and organizes this scar-based wound healing is poorly understood (O′Shea, T.M. et al., Cell biology of spinal cord injury and repair. The Journal of clinical investigation 127, 3259-3270, doi:10.1172/JCI90608 (2017); Stenudd, M., Sabelstrom, H. & Frisen, J. Role of endogenous neural stem cells in spinal cord injury and repair. JAMA Neurol 72, 235-237, doi:10.1001/jamaneurol.2014.2927 (2015); Jin, Y. & Zheng, B. Multitasking: Dual Leucine Zipper-Bearing Kinases in Neuronal Development and Stress Management. Annual review of cell and developmental biology 35, 501-521, doi:10.1146/annurev-cellbio-100617-062644 (2019)). Furthermore, the role of reactive glia in axon regeneration failure remains an open question because mature neurons also exhibit diminished intrinsic regenerative capacity in the adult (He, Z. & Jin, Y. Intrinsic Control of Axon Regeneration. Neuron 90, 437-451, doi:10.1016/j.neuron.2016.04.022 (2016); Hutson, T. H. et al. Cbp-dependent histone acetylation mediates axon regeneration induced by environmental enrichment in rodent spinal cord injury models. Science translational medicine 11, doi:10.1126/scitranslmed.aaw2064 (2019)). To address these questions, spinal cord regeneration was examined in neonatal mice, a developmental stage when CNS neurons still have high growth ability.

### Example 2: Axon Passing Across Neonatal Injury

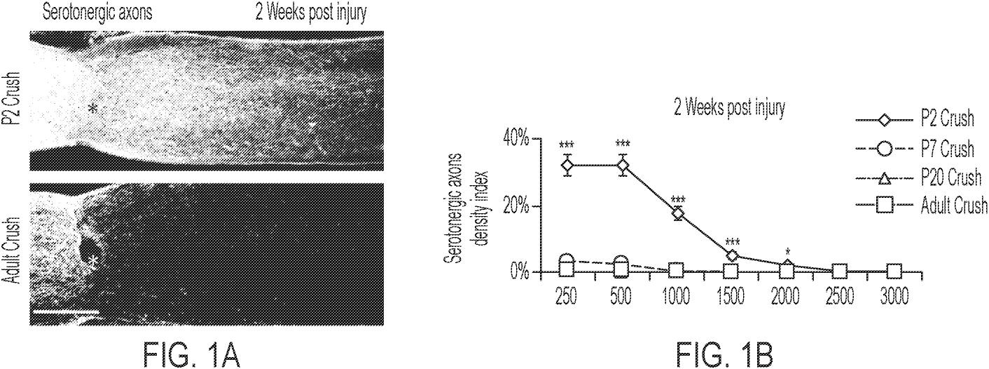

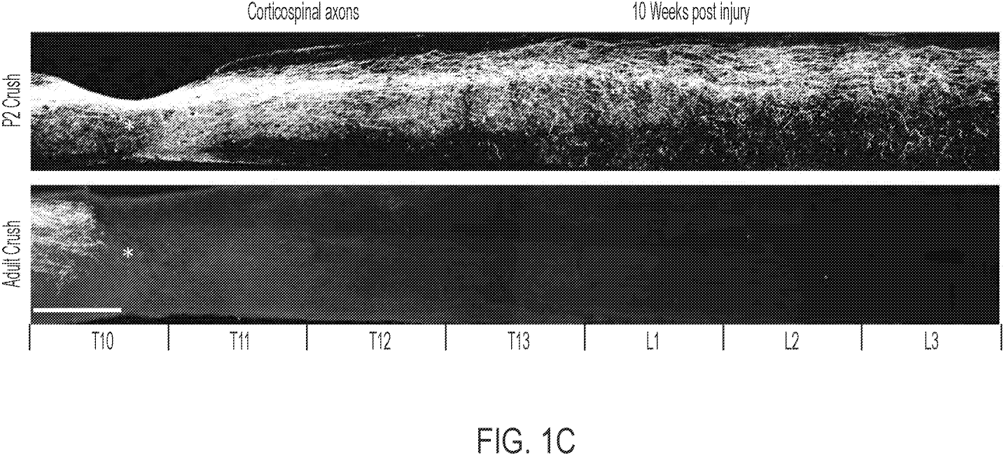

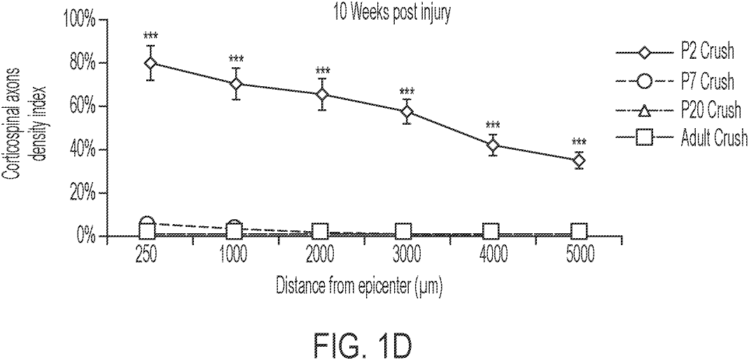

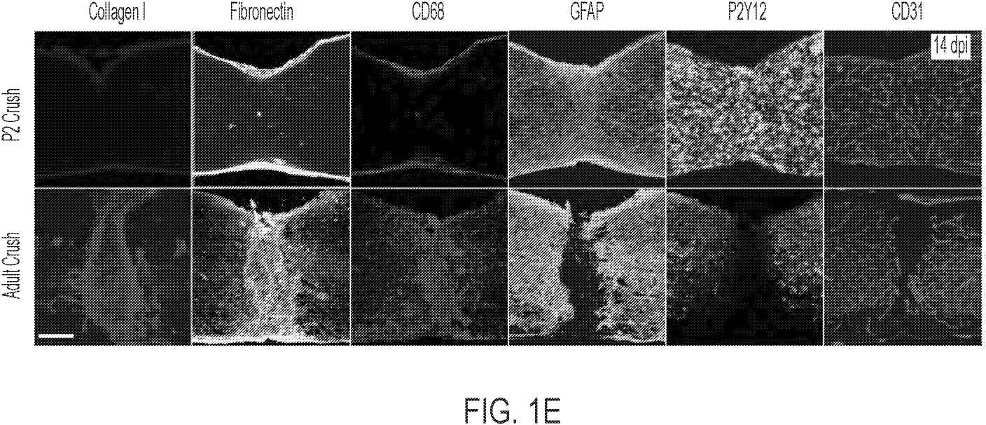

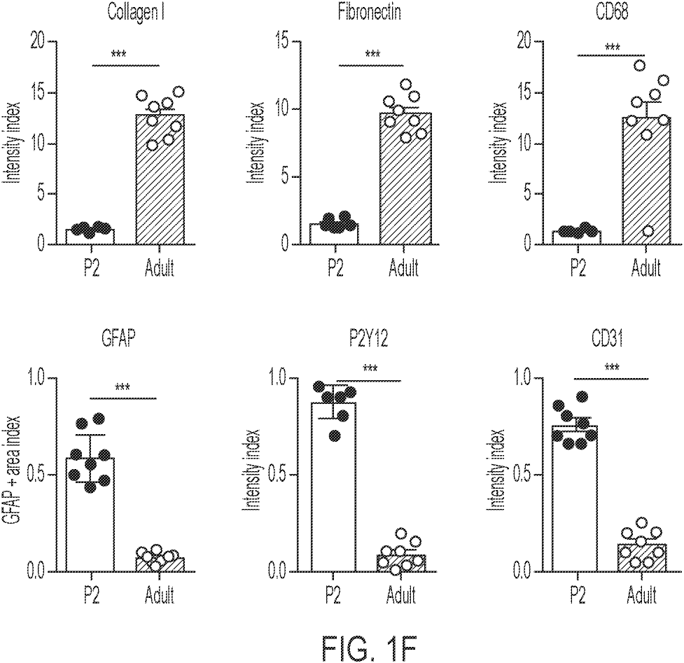

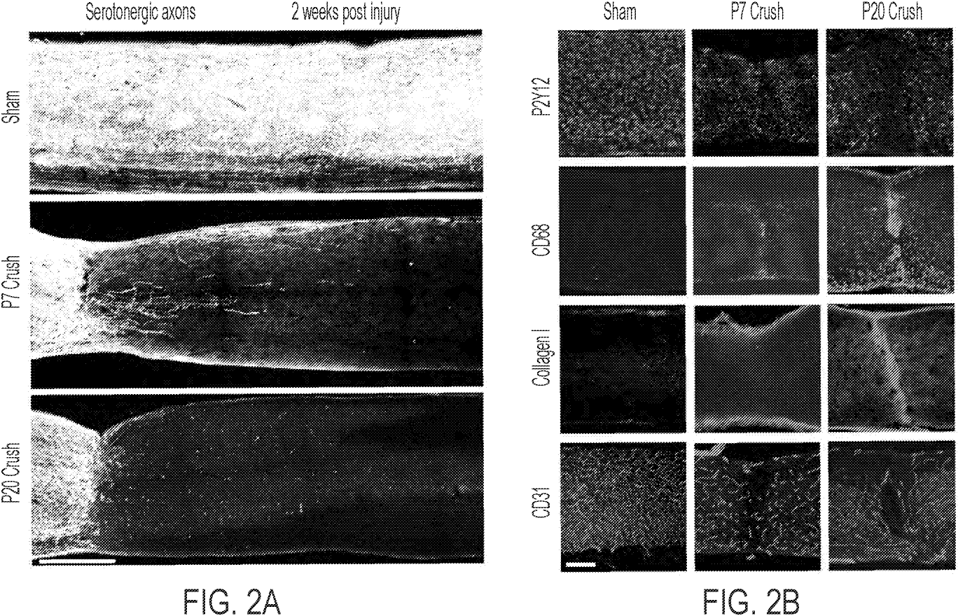

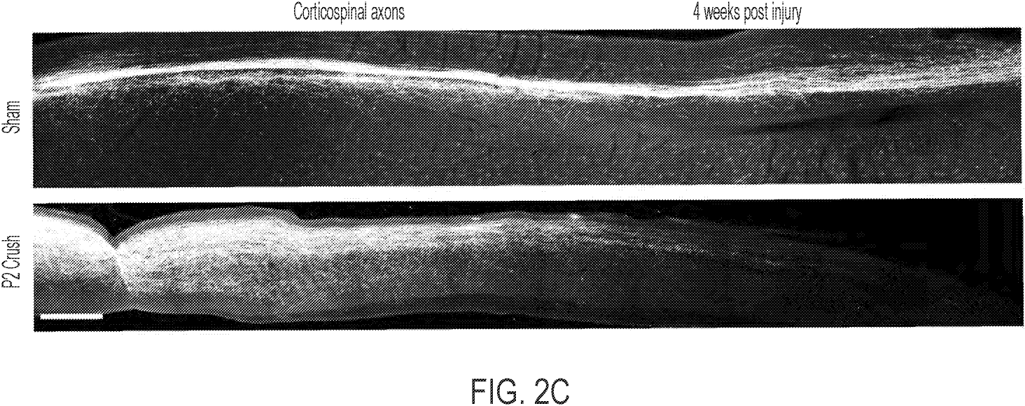

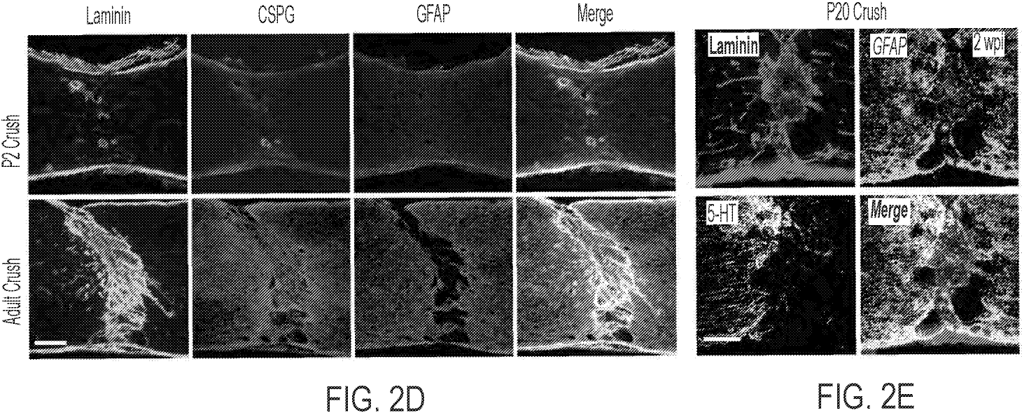

To assess age-dependent differences in axon regrowth, spinal cord crush injuries were performed in wild type mice of different developmental stages, including postnatal day 2 (P2), day 7 (P7), juvenile day 20 (P20) and adult (FIGS. 1A-1F, 2A). At two weeks after injury in P20 or adult mice, few serotonergic axons were observed in the spinal cord distal to the lesion sites (FIGS. 1A, 1B, 2A). In contrast, two weeks after P2 crush injury, numerous serotonergic axons, positively stained with antibodies against 5-HT (Bregman, B. S. Spinal cord transplants permit the growth of serotonergic axons across the site of neonatal spinal cord transection. Brain research 431, 265-279, doi:10.1016/0165-3806(87)90214-8 (1987)), extended beyond the lesion site (FIGS. 1A and 1B). This robust growth was not unique to serotonergic axons, as corticospinal axons also reached into the lumbar spinal cord at 4-10 weeks after injury (FIGS. 1C, 1D, 2C). In contrast to P2, only a few axons passed the lesion after crush injury at P7 (FIGS. 1B, 2A). These results suggest that only after an early neonatal (P2) injury, a permissive environment for axon growth is established.

### Example 3: Scar-Free Healing After Neonatal Injury

The characteristics of lesion sites after P2 versus adult crush injuries were compared. At 2 weeks after adult injury, a typical scar structure formed in the center of lesions with a significant accumulation of CD68+ cells (activated macrophages/microglia (Bellver-Landete, V. et al. Microglia are an essential component of the neuroprotective scar that forms after spinal cord injury. Nature communications 10, 518, doi:10.1038/s41467-019-08446-0 (2019); Schafer, D. P. et al. Microglia sculpt postnatal neural circuits in an activity and complement-dependent manner. Neuron 74, 691-705, doi:10.1016/j.neuron.2012.03.026 (2012))) surrounded by fibroblasts, reactive astrocytes and basal lamina components stained with collagen I, fibronectin, CSPG and laminin (FIGS. 1E, 1F, 2D, and 2E). In contrast, the injury site at the same post-injury time point (2 weeks) after P2 injury was different, with minimal accumulation of CD68+ cells, fibronectin+ and collagen I+, CSPG or laminin+ basal lamina, but with modest accumulation of GFAP+ cells in the lesion center (FIGS. 1E, 1F). P2Y12, an established marker for homeostatic microglia (Butovsky, O. et al. Identification of a unique TGF-beta-dependent molecular and functional signature in microglia. Nature neuroscience 17, 131-143, doi:10.1038/nn.3599 (2014)), was absent in the adult lesions, whereas P2Y12+ cells populated the P2 lesions and were evenly distributed throughout the injury site (FIGS. 1E, 1F).

Furthermore, immunostaining with anti-CD31 antibodies revealed a continuous vasculature across the P2-lesioned site, in a pattern indistinguishable from the intact spinal cord. The characteristics of the P20 lesions were similar to those in adult injuries, while P7 lesions had reduced scar formation (FIG. 2B). Thus, the neonatal spinal cord was able to mount a scar-free wound healing response, thereby permitting descending axons to grow across the lesion.

### Example 4: Re-Establishing Microglia Homeostasis

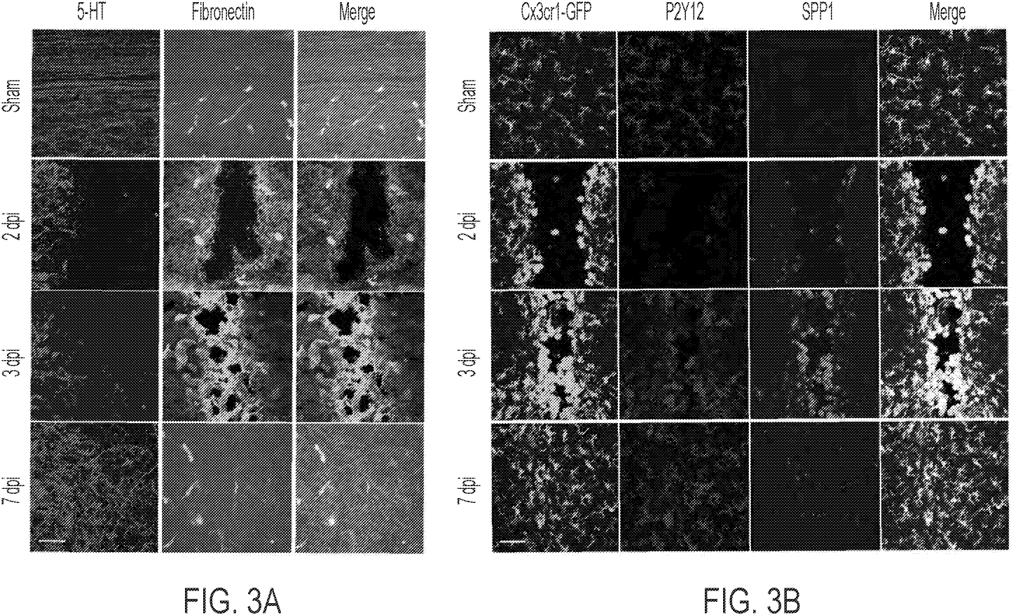

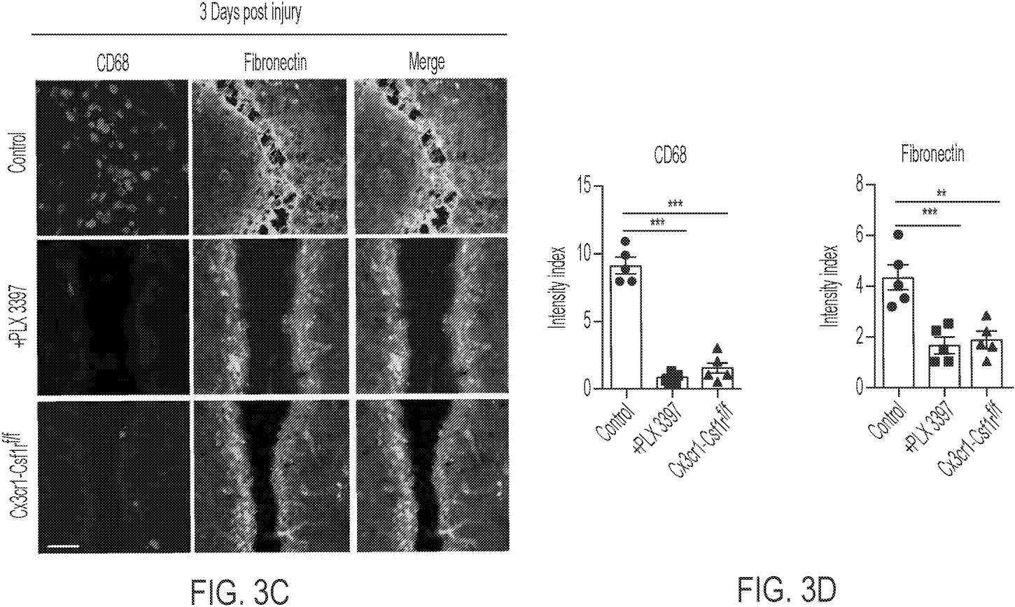

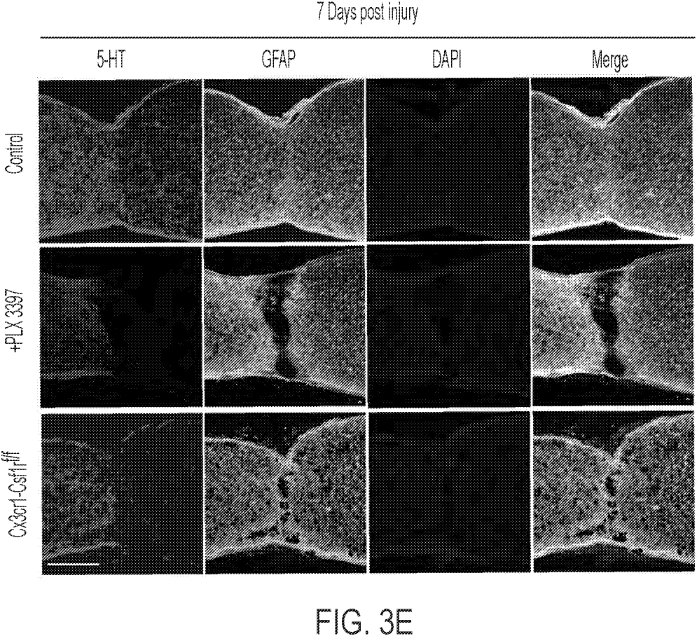

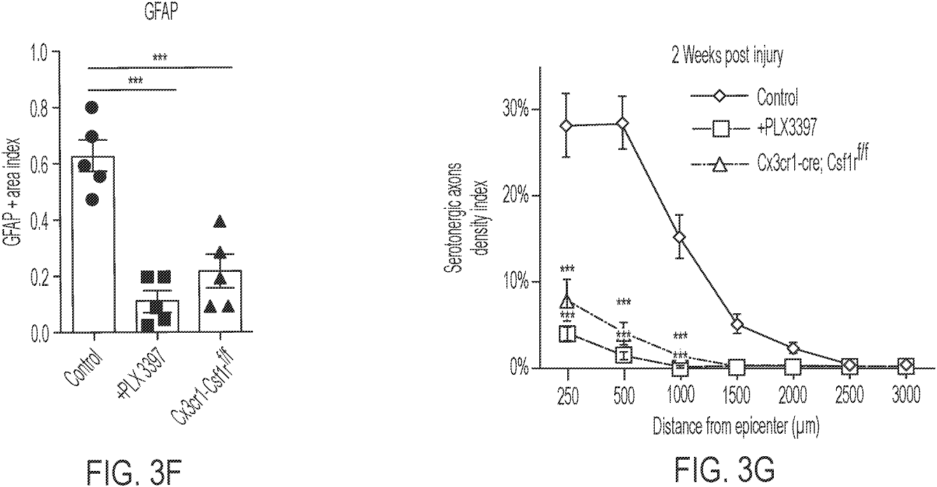

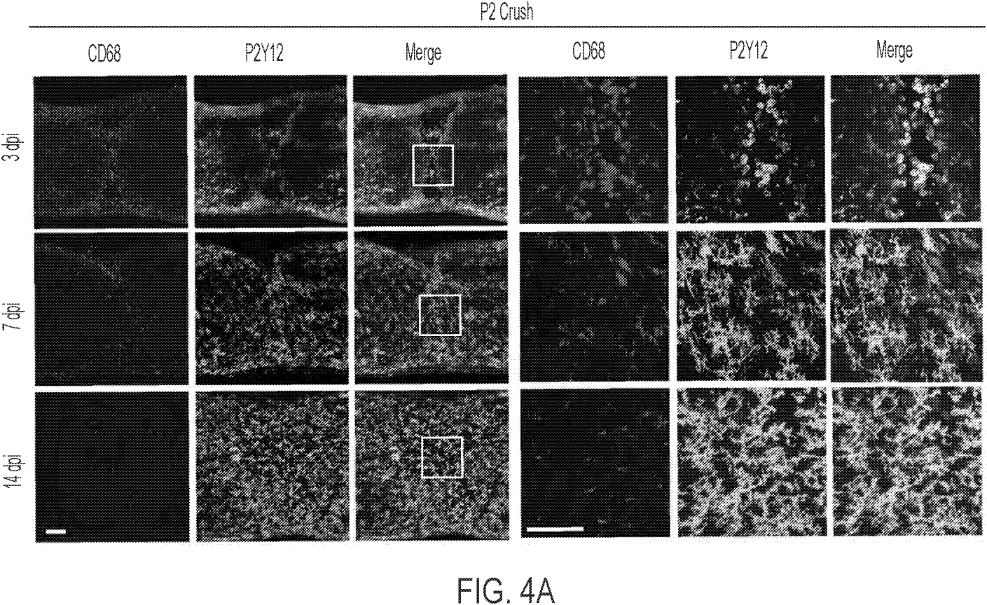

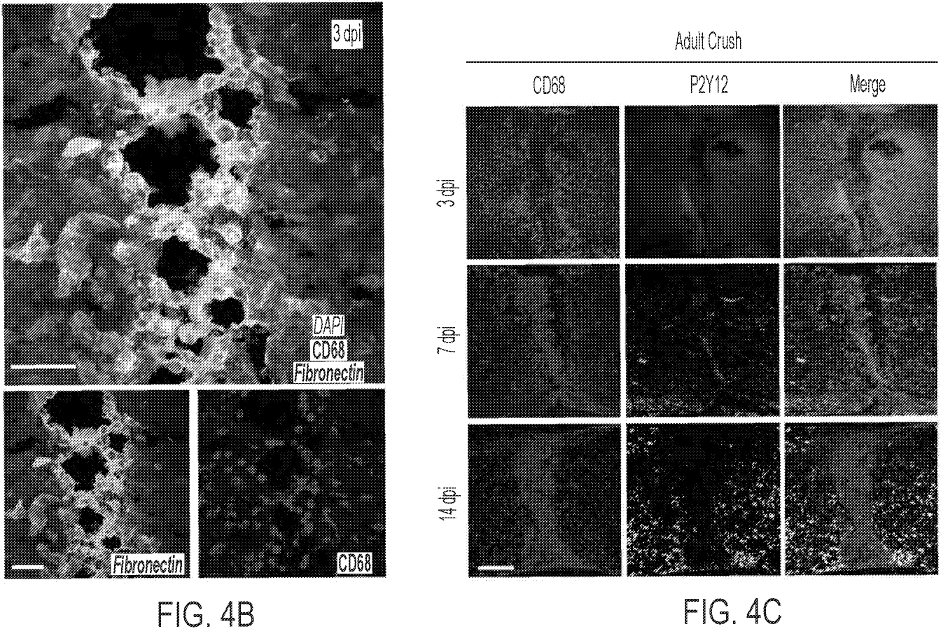

To characterize the dynamics of neonatal wound healing process, the lesion site was examined at early time points after P2 injury. At 1-2 days post injury (dpi), a clear gap was present between the two stumps of cut ends and serotonergic axons stopped rostral to the lesion (FIG. 3A). By 3 dpi, fibronectin+ cells appeared between the gap (FIGS. 3A, 4B), and by 7 dpi, the gap was repaired, fibronectin signal disappeared and serotonergic axons grew into and across the lesion. To determine the source of fibronectin+ cells populating the gap at 3 dpi, Cx3cr1-GFP mice, in which microglia are labeled with GFP (Jung, S. et al. Analysis of fractalkine receptor CX(3)CR1 function by targeted deletion and green fluorescent protein reporter gene insertion. Molecular and cellular biology 20, 4106-4114, doi:10.1128/mcb.20.11.4106-4114.2000 (2000)), were used. Microglia accumulated in the stumps at 2 dpi. At this point, they lost the expression of P2Y12, and instead expressed activated microglia markers such as SPP1+ and CD68+ (FIGS. 3B, 4A-4C).

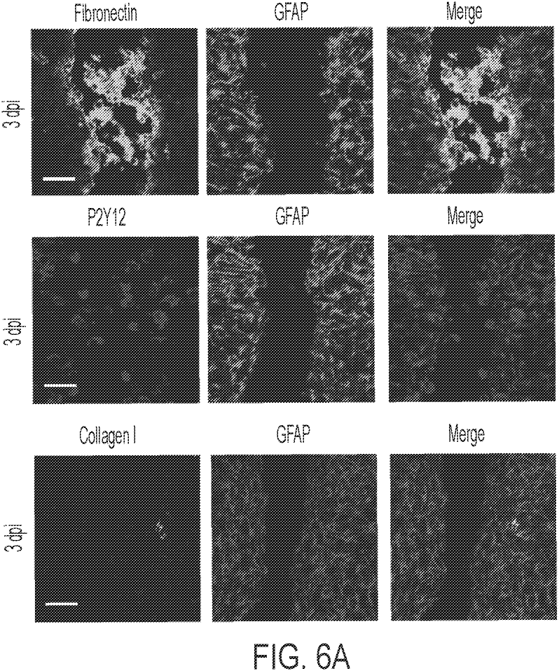

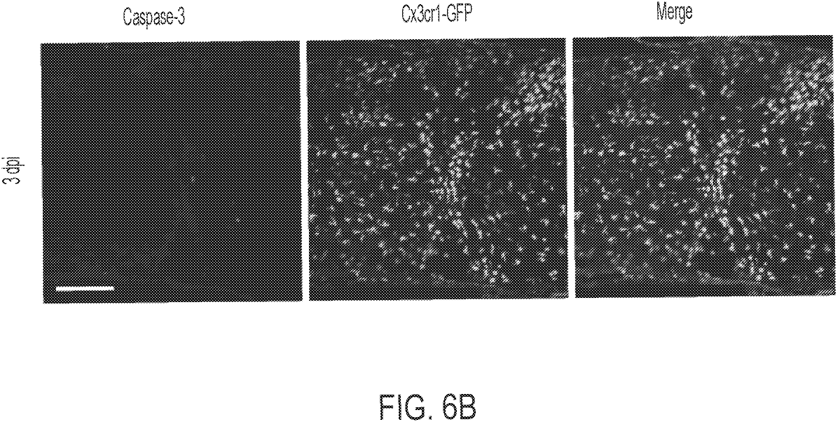

Morphologically, these microglia transformed from highly ramified (homeostatic) to amoeboid (activated) shape (FIGS. 3B, 4A), consistent with injury induced microglia activation (Hammond, T. R. et al. Single-Cell RNA Sequencing of Microglia throughout the Mouse Lifespan and in the Injured Brain Reveals Complex Cell-State Changes. Immunity 50, 253-271 e256, doi:10.1016/j.immuni.2018.11.004 (2019)). Starting from 3 dpi when fibronectin+ bridges were forming between the two stumps, activated microglia were observed inside the lesion in the absence of GFAP+ astrocytes and collagen I+ fibroblasts (FIG. 6A). These cells began to regain P2Y12 expression, but also were still positively stained with SPP1 and CD68 (FIGS. 3B, 4A). Around 7 dpi, most microglia displayed a ramified morphology and were positive for P2Y12 and negative for SPP1 and CD68 (FIGS. 3B, 4A). In combination with the observations of minimal cell death (FIG. 6B) and limited time for cell division, these results indicated that after P2 injury, microglia became transiently activated, and then returned to a homeostatic state within the first 7 days.

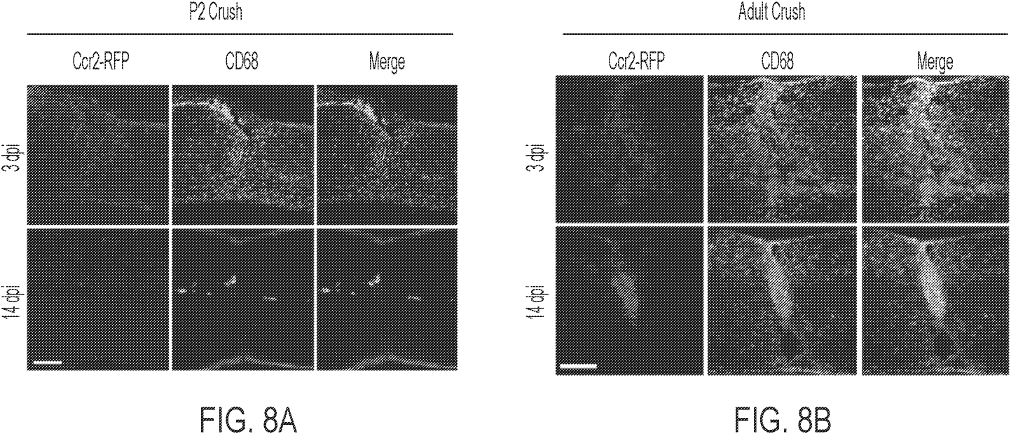

Accompanying this rapid microglial reaction is the disappearance of intra-lesional fibronectin by 7 dpi (FIGS. 1E, 3A). Conversely, fibronectin persisted in adult lesions (FIG. 1E). A hallmark feature of the injured adult spinal cord is the accumulation and persistence of blood monocyte-derived macrophages in the lesion (Popovich, P. G. & Hickey, W. F. Bone marrow chimeric rats reveal the unique distribution of resident and recruited macrophages in the contused rat spinal cord. Journal of neuropathology and experimental neurology 60, 676-685, doi:10.1093/jnen/60.7.676 (2001); Zhu, Y. et al. Hematogenous macrophage depletion reduces the fibrotic scar and increases axonal growth after spinal cord injury. Neurobiology of disease 74, 114-125, doi:10.1016/j.nbd.2014.10.024 (2015)). After P2 injury, CCR2-RFP+ monocyte-derived macrophages (Saederup, N. et al. Selective chemokine receptor usage by central nervous system myeloid cells in CCR2-red fluorescent protein knock-in mice. PloS one 5, e13693, doi:10.1371/journal.pone.0013693 (2010)) accumulated in the lesion within 3d, but were absent by 14 days (FIG. 8A)). In contrast, in adult spinal lesions, monocyte-derived macrophages persisted in the lesions and activated microglia continuously expressed CD68 without re-expressing P2Y12 (FIGS. 4C, 8B). Therefore, there was a transient accumulation of blood-derived macrophages in neonatal, but not adult, spinal cord injury sites.

### Example 5: Efficient Healing Requires Microglia

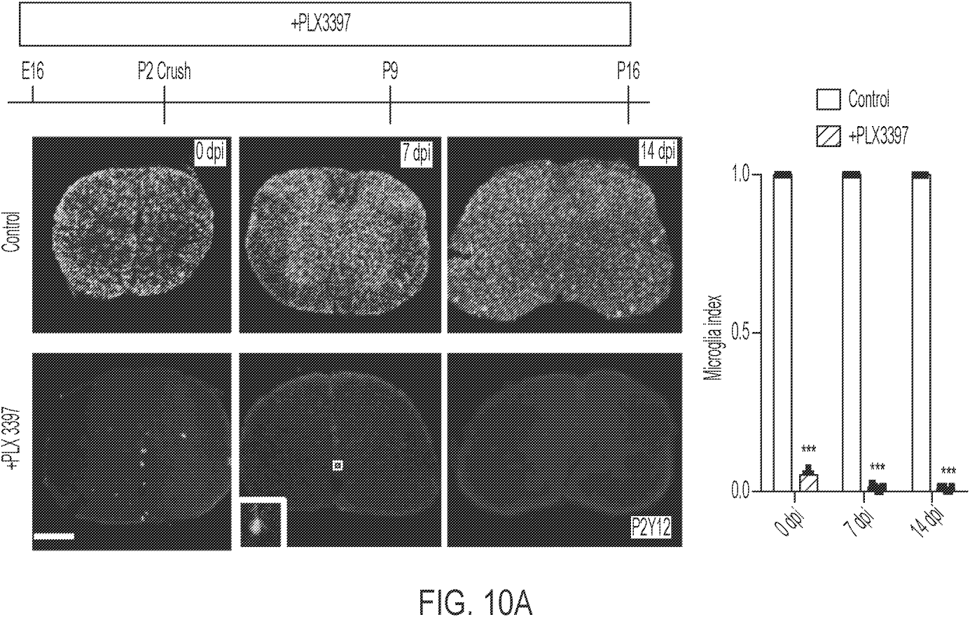

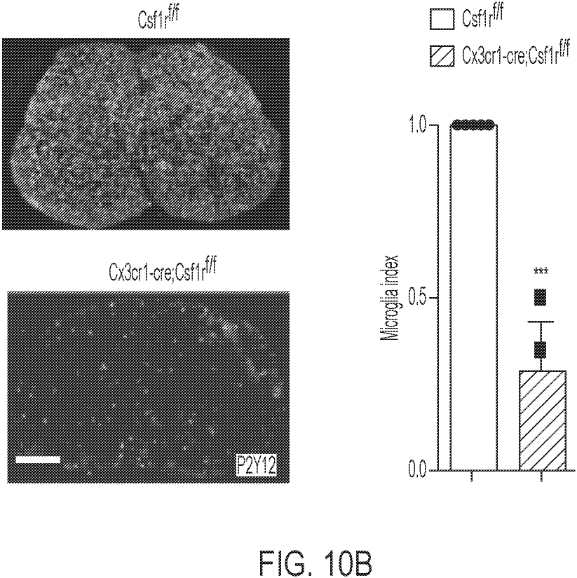

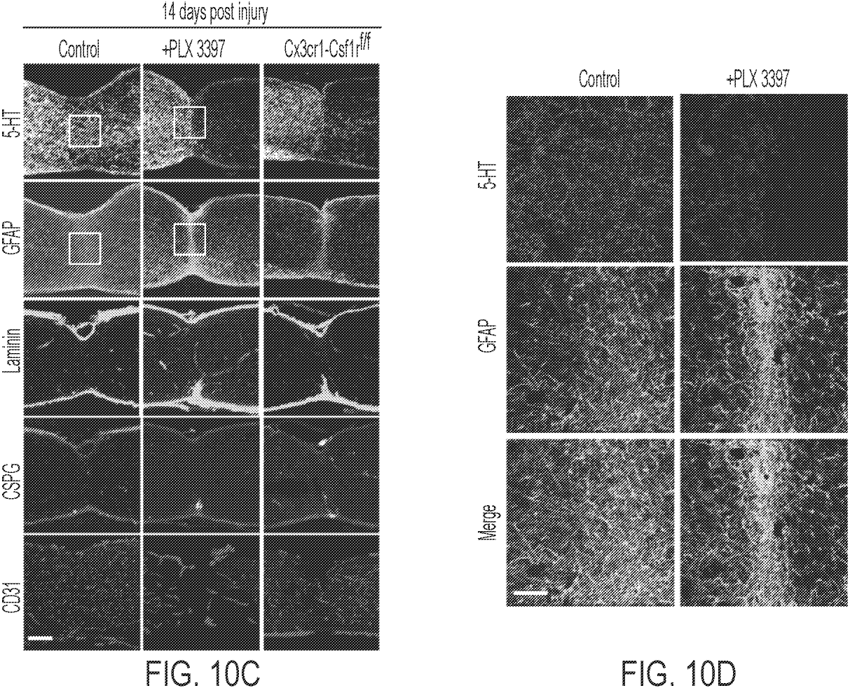

In order to further assess the functional role of microglia in scar-free wound healing in neonatal spinal cord, PLX 3397, a colony-stimulating factor 1 receptor (CSF1R) inhibitor (Elmore, M. R. et al. Colony-stimulating factor 1 receptor signaling is necessary for microglia viability, unmasking a microglia progenitor cell in the adult brain. Neuron 82, 380-397, doi:10.1016/j.neuron.2014.02.040 (2014)), was used to deplete microglia in vivo. 7 days after PLX3397 treatment, most microglia were depleted in neonatal spinal cord (FIG. 10A). Microglia depletion in P2 injury model impaired bridge formation between the two severed stumps at 3 or 7 dpi (FIGS. 3C-3F). At 14 dpi, the gap was closed with strong GFAP+ reactive astrocytes accumulating in the epicenter, and blood vessels were absent from the “astroglial scar” (FIGS. 3G, 10C, 10D). However, limited accumulations of basal lamina components, such as CSPG and laminin, were detected (FIG. 10C); supporting that swirls of basal lamina structures formed after lesions in the mature, but not the neonatal, cortex (Rudge, J. S. & Silver, J. Inhibition of neurite outgrowth on astroglial scars in vitro. The Journal of neuroscience : the official journal of the Society for Neuroscience 10, 3594-3603 (1990)). The majority of axons stalled at the lesion epicenter, with only a few penetrating into GFAP+ cells, suggesting a lack of inhibitory basal lamina components (McKeon, R. J., et al. Reduction of neurite outgrowth in a model of glial scarring following CNS injury is correlated with the expression of inhibitory molecules on reactive astrocytes. The Journal of neuroscience : the official journal of the Society for Neuroscience 11, 3398-3411 (1991)). To further verify the pharmacological depletion results, P2 crush injury was applied in mice with conditional knockout of CSF1R in microglia (Cx3cr1-Cre crossed with Csf1r^{f/f} (Yona, S. et al. Fate mapping reveals origins and dynamics of monocytes and tissue macrophages under homeostasis. Immunity 38, 79-91, doi:10.1016/j.immuni.2012.12.001 (2013); Waisman, A., et al. Homeostasis of Microglia in the Adult Brain: Review of Novel Microglia Depletion Systems. Trends Immunol 36, 625-636, doi:10.1016/j.it.2015.08.005 (2015))), which removed about 70% of microglia (FIG. 10B). Similar findings were observed (FIGS. 3C-3G, 10C). Thus, neonatal microglia are essential for achieving scar-free wound healing responses after P2 injury.

### Example 6: Signature of Repair-Promoting Microglia

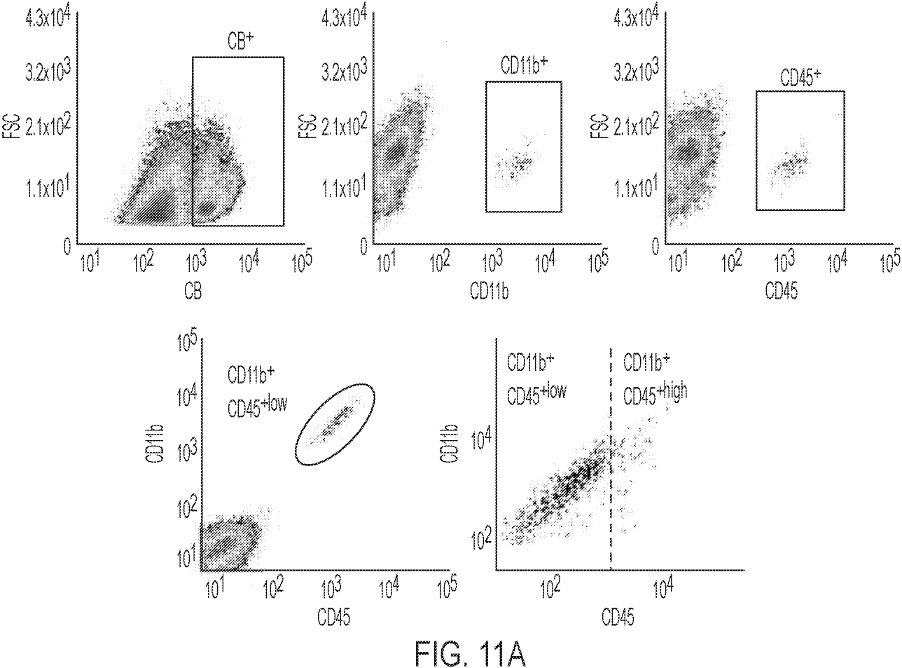

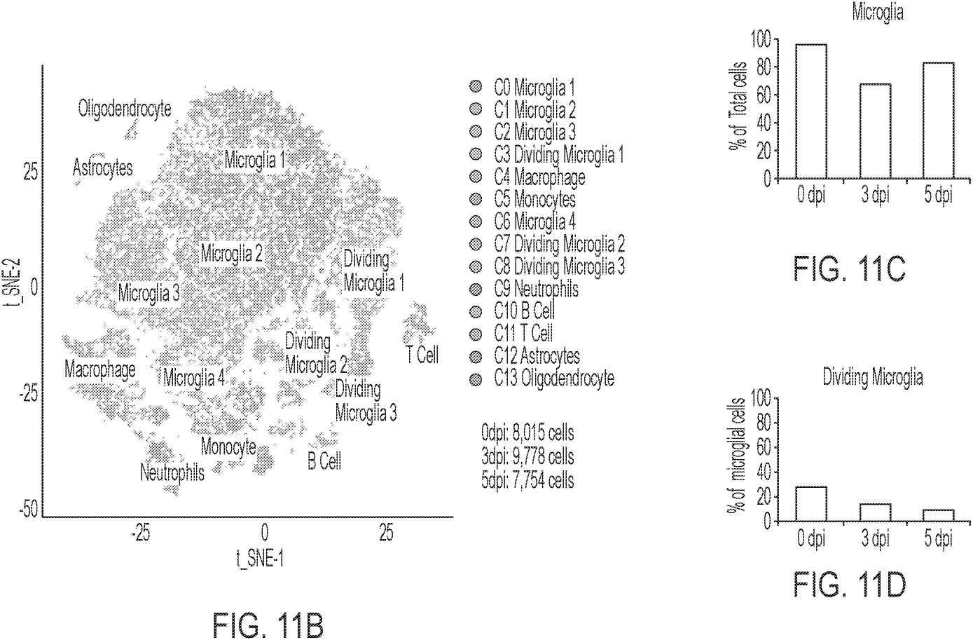

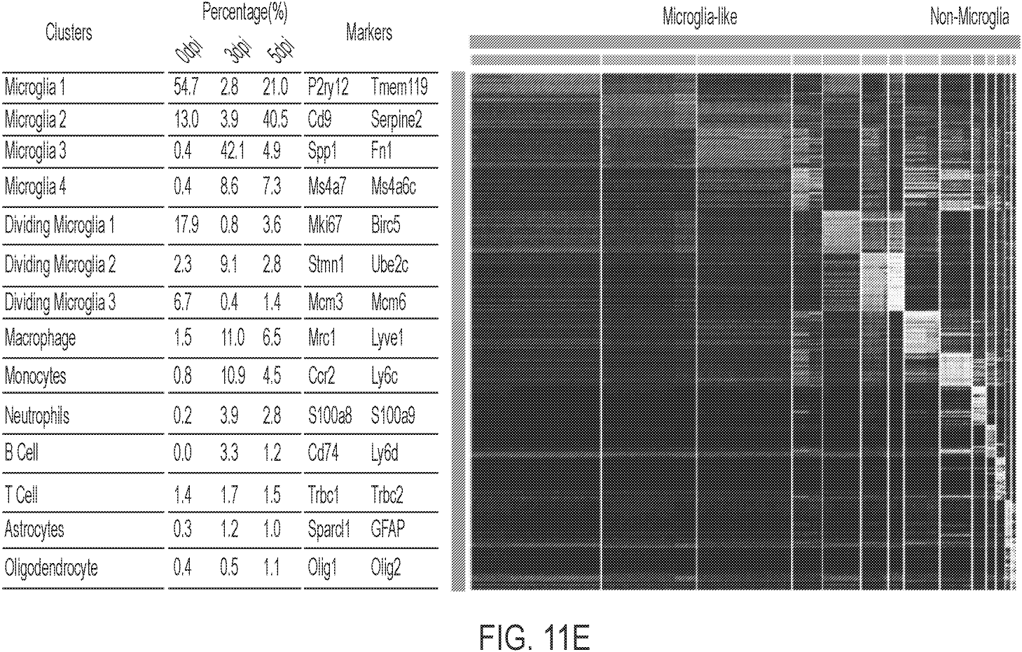

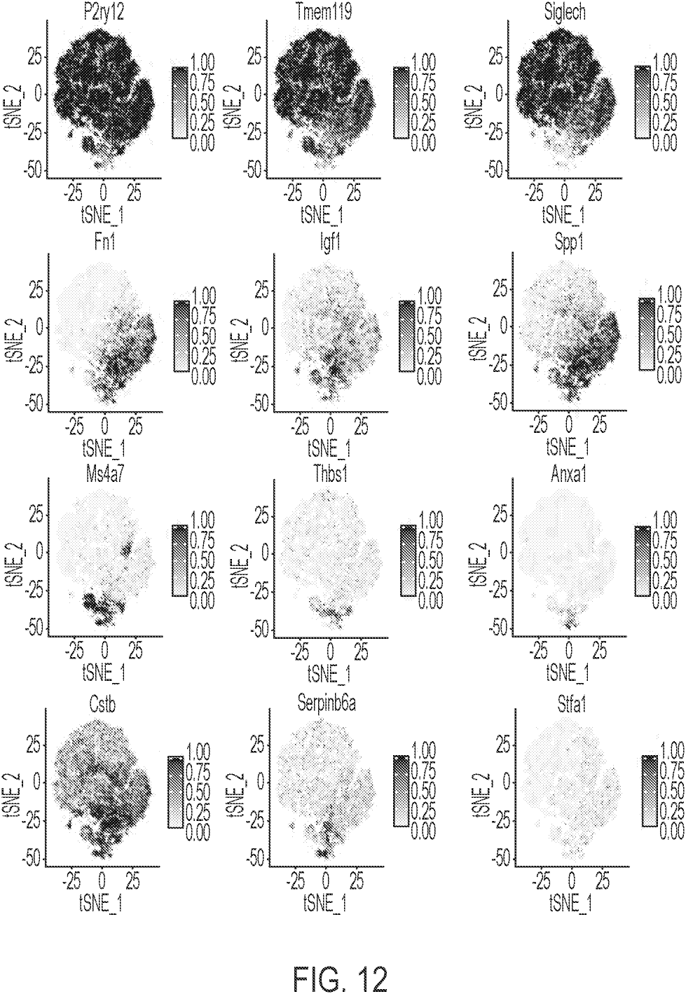

To evaluate the neonatal microglia-mediated responses to injury (during the first week), single cell RNA-seq analysis (scRNA-Seq) was conducted. At different times after P2 injury (0, 3, and 5 dpi), spinal cord tissues (2 mm including crush site) were dissected. Dissociated cells were FACS sorted with CD11b; CD45^{low} gating (FIG. 11A) and the isolated cells were subjected to 10x scRNA-Seq. Unsupervised clustering using t-distributed stochastic neighbor embedding (t-SNE) (Macosko, E. Z. et al. Highly Parallel Genome-wide Expression Profiling of Individual Cells Using Nanoliter Droplets. Cell 161, 1202-1214, doi:10.1016/j.cell.2015.05.002 (2015)) revealed 14 clusters with distinct gene expression signatures (FIG. 11B). Gene ontology analysis classified these clusters into discrete subpopulations, including non-dividing microglia cells (C0, C1, C2 and C6), dividing-microglia cells (C3, C7 and C8), macrophages (C4), monocytes (C5), neutrophils (C9), B cells (C10), T cells (C11), astrocytes (C12), and oligodendrocytes (C13) (FIGS. 11B, 11C, 11E).

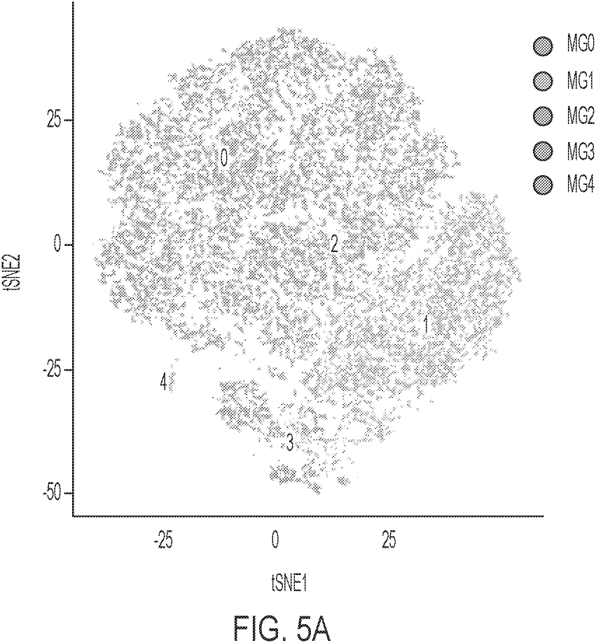

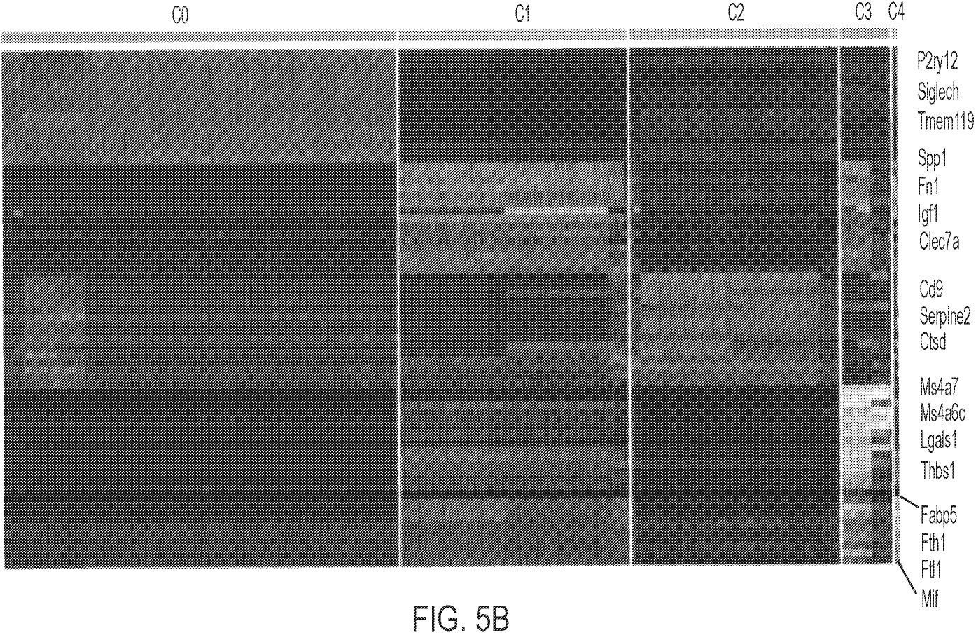

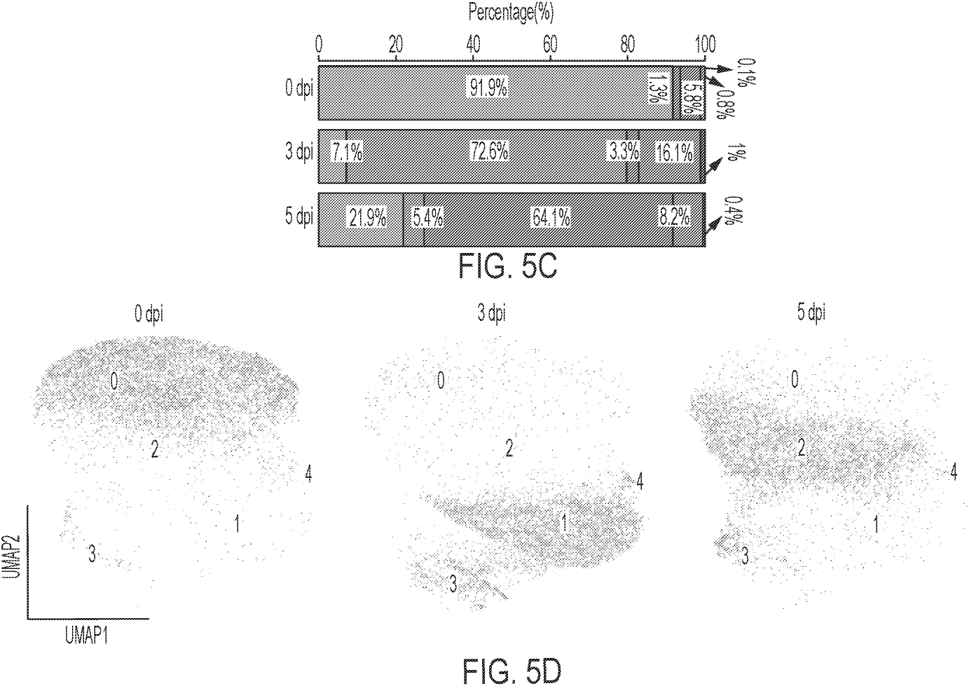

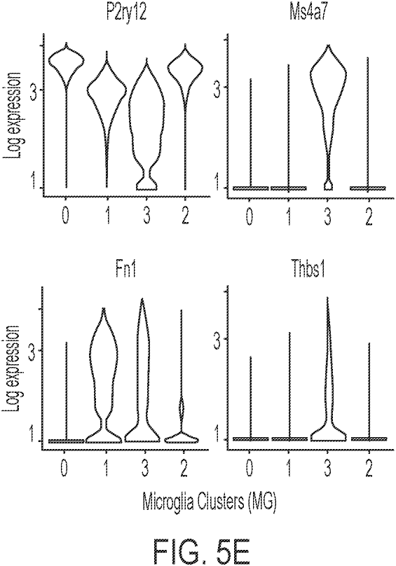

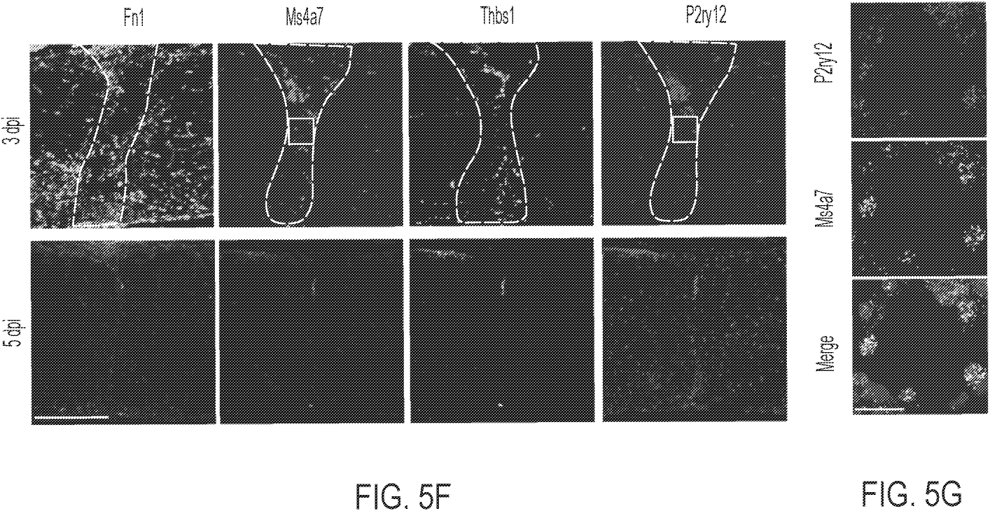

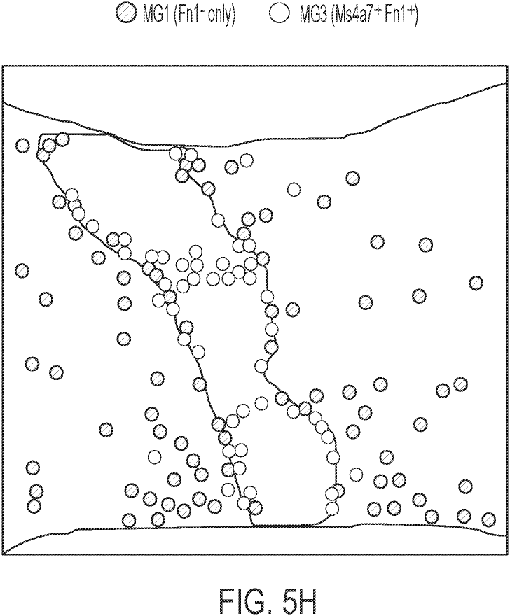

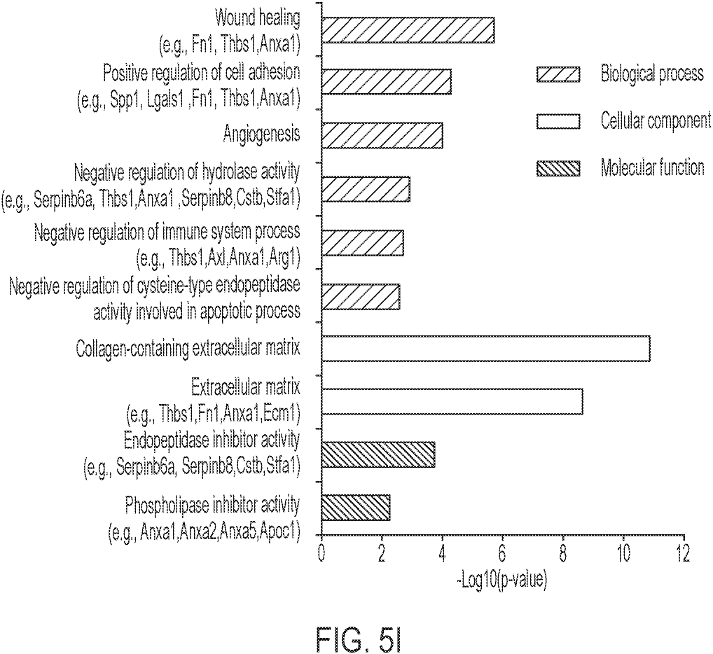

Among the microglia analyzed, 28.2% at 0 dpi were dividing, this number reduced to 15.1% at 3 dpi and 9.5% at 5 dpi (FIG. 11D), suggesting that microglial proliferation gradually declined in the lesion site after injury. As cell cycle genes can overload the major principal components that underlie cell-to-cell variations (Buettner, F. et al. Computational analysis of cell-to-cell heterogeneity in single cell RNA-sequencing data reveals hidden subpopulations of cells. Nature biotechnology 33, 155-160, doi:10.1038/nbt.3102 (2015); Li, Q. et al. Developmental Heterogeneity of Microglia and Brain Myeloid Cells Revealed by Deep Single-Cell RNA Sequencing. Neuron 101, 207-223 e210, doi:10.1016/j.neuron.2018.12.006 (2019)), cell cycle effects were regressed out and all microglia cells were re-clustered (dividing and non-dividing microglia, 20,542 cells in total) into five transcriptionally distinct cell clusters: MG0-MG4 (FIGS. 5A-5E). These cells represented different states of the same cell type, but some cells could represent new cells arising by mitosis or migrating from elsewhere. Most microglia (91.9%) in intact spinal cord were in the cluster MG0, which expressed homeostatic microglia marker genes, such as P2ry12, Tmem119 and Siglech. At 3 dpi, the majority of microglia fell into two new states MG1 (72.6%) and MG3 (16.1%). Both MG1 and MG3 cells expressed typical microglial activation markers (e.g., Spp1, Igf1) (Hammond, T. R. et al. Single-Cell RNA Sequencing of Microglia throughout the Mouse Lifespan and in the Injured Brain Reveals Complex Cell-State Changes. Immunity 50, 253-271 e256, doi:10.1016/j.immuni.2018.11.004 (2019); Li, Q. et al. Developmental Heterogeneity of Microglia and Brain Myeloid Cells Revealed by Deep Single-Cell RNA Sequencing. Neuron 101, 207-223 e210, doi:10.1016/j.neuron.2018.12.006 (2019)), but MG3 expressed additional genes such as Ms4a7 (FIG. 12), which is known to be expressed in embryonic microglia (Hammond, T. R. et al. Single-Cell RNA Sequencing of Microglia throughout the Mouse Lifespan and in the Injured Brain Reveals Complex Cell-State Changes. Immunity 50, 253-271 e256, doi:10.1016/j .immuni.2018.11.004 (2019)) and border macrophages (Mrdjen, D. et al. High-Dimensional Single-Cell Mapping of Central Nervous System Immune Cells Reveals Distinct Myeloid Subsets in Health, Aging, and Disease. Immunity 48, 380-395 e386, doi:10.1016/j.immuni.2018.01.011 (2018); Jordao, M. J. C. et al. Single-cell profiling identifies myeloid cell subsets with distinct fates during neuroinflammation. Science 363, doi:10.1126/science.aat7554 (2019)). A fraction of microglia (MG4), which accounted for less than 1% after injury, exhibited an embryonic metabolic profile with expression of Fabp5 and Mif (Hammond, T. R. et al. Single-Cell RNA Sequencing of Microglia throughout the Mouse Lifespan and in the Injured Brain Reveals Complex Cell-State Changes. Immunity 50, 253-271 e256, doi:10.1016/j.immuni.2018.11.004 (2019)). At 5 dpi, microglia were either in the cluster MG2 (64.1%) or in homeostatic MG0 (21.9%). MG2 cells expressed activation genes at lower levels and were phenotypically between MG1 and MG0 on the UMAP plot (FIG. 5D), supporting that in initially activated microglia, both MG1 and MG3, were rapidly transitioning back to the homeostatic stage at this time point.

To examine the expression patterns of several top-ranked markers in each cluster (FIG. 5E) in the spinal cord around lesion site (FIGS. 5F, 5G), in situ hybridization was performed. Fnl encoding fibronectin, expressed in both MG1 and MG3, but not MG2, was expressed by the cells in and around the lesion at 3 dpi, but not 5 dpi. In contrast, Ms4a7+ and Thbs1+, two unique genes expressed in MG3 cells, were concentrated in the microglia immediately in the lesion and in the bridge only at 3 dpi (FIGS. 5F, 5G). Thus, MG1 and MG3 cells exhibited a complementary distribution, with MG3 cells immediately in and around the lesion and MG1 cells in the spinal cord around the lesion (FIG. 5H).

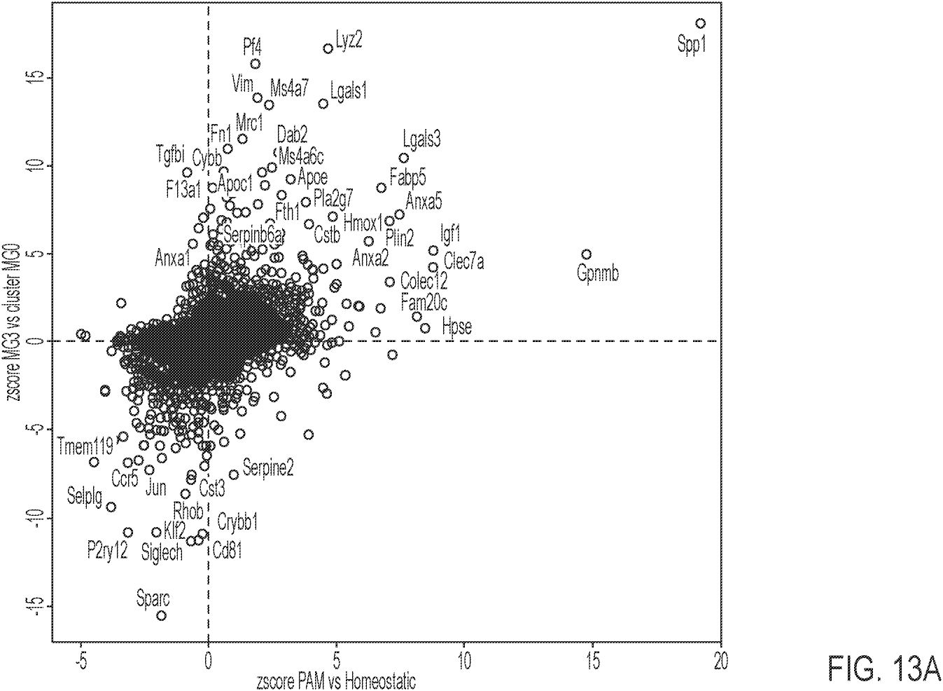

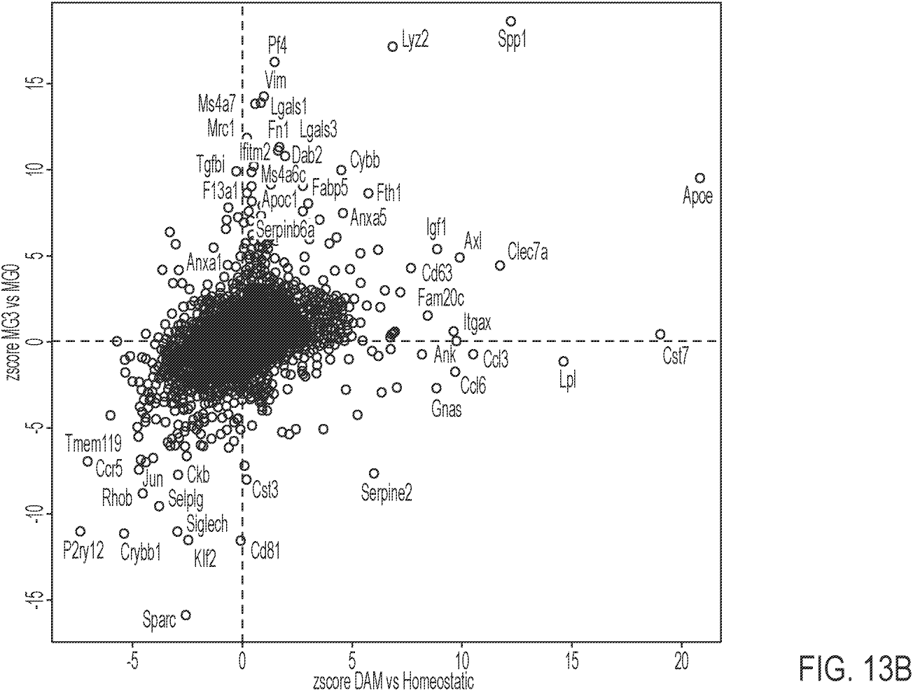

The molecular signatures of MG3 were then evaluated, because of their proximity to the lesion. These cells expressed several genes enriched in proliferative-region associated microglia (PAM) (Hammond, T. R. et al. Single-Cell RNA Sequencing of Microglia throughout the Mouse Lifespan and in the Injured Brain Reveals Complex Cell-State Changes. Immunity 50, 253-271 e256, doi:10.1016/j.immuni.2018.11.004 (2019); Li, Q. et al. Developmental Heterogeneity of Microglia and Brain Myeloid Cells Revealed by Deep Single-Cell RNA Sequencing. Neuron 101, 207-223 e210, doi:10.1016/j.neuron.2018.12.006 (2019)) and disease-associated microglia (DAM) (Keren-Shaul, H. et al. A Unique Microglia Type Associated with Restricting Development of Alzheimer’s Disease. Cell 169, 1276-1290 e1217, doi:10.1016/j.cell.2017.05.018 (2017)), such as Spp1, Igf1, Clec7a (Li, Q. et al. Developmental Heterogeneity of Microglia and Brain Myeloid Cells Revealed by Deep Single-Cell RNA Sequencing. Neuron 101, 207-223 e210, doi:10.1016/j.neuron.2018.12.006 (2019)), and embryonic microglia (Hammond, T. R. et al. Single-Cell RNA Sequencing of Microglia throughout the Mouse Lifespan and in the Injured Brain Reveals Complex Cell-State Changes. Immunity 50, 253-271 e256, doi:10.1016/j.immuni.2018.11.004 (2019)), such as Ms4a7, Ms4a6c, Lgals1 (FIG. 12), suggesting that neonatal injury converted homeostatic microglia into a state with both activation and de-differentiation signatures. However, these MG3 cells exhibited unique gene expression (FIGS. 13A-13B). By gene ontology (GO) analysis of the top 300 ranked genes in MG3 cluster (FIGS. 5I, 14A), a significant enrichment of functions in extracellular matrix (ECM), wound healing, phagocytosis, angiogenesis, and negative regulation of immune responses and endopeptidases was discovered (FIG. 5I). Consistently, analysis of the potential gene regulatory network indicated up-regulation of the genes involved in ECM (e.g., fibronectin 1 or Fn1 and thrombospondin 1 or Thbs1) and anti-inflammatory genes (e.g., serine-type and cysteine type endopeptidase inhibitor activity, phospholipase A2 inhibitor activity) in MG3 microglia cells (FIG. 5I). For example, Fn1, together with Thbs1, an extracellular matrix molecule that can bind to fibronectin (Tan, K. & Lawler, J. The interaction of Thrombospondins with extracellular matrix proteins. J Cell Commun Signal 3, 177-187, doi:10.1007/s12079-009-0074-2 (2009)), were highly expressed in MG3, indicating a role of MG3 in bridge formation. MG3 cells also had enriched expression of the inhibitors of peptidases and endopeptidases, such as Cstb, Stfa1 and Serpinb6a, as well as Anxa1, a pro-resolving mediator, which may have contributed to their rapid inflammation resolution. These genes exhibited different expressions to the microglia isolated after adult spinal cord injury: adult microglia have persistent expression of Fn1 and no significant induction of proteinase inhibitors (FIGS. 1E, 14B, 14C). Together, these results indicate that injury-induced neonatal MG3 microglia have unique molecular properties suited for promoting scar-free wound healing.

### Example 7: Bridge Forming by Microglia-Derived Fn1

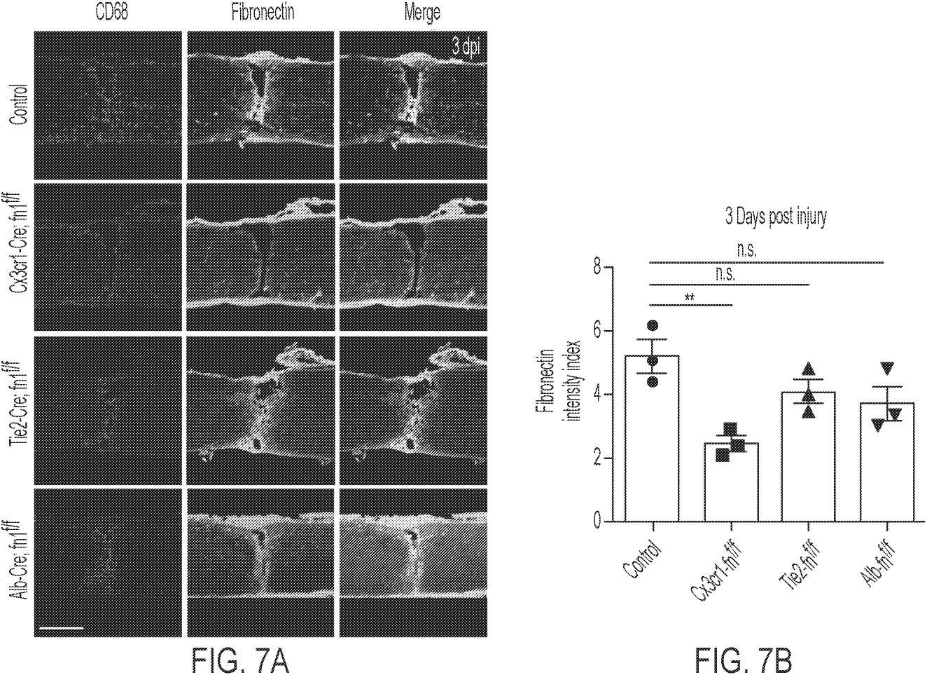

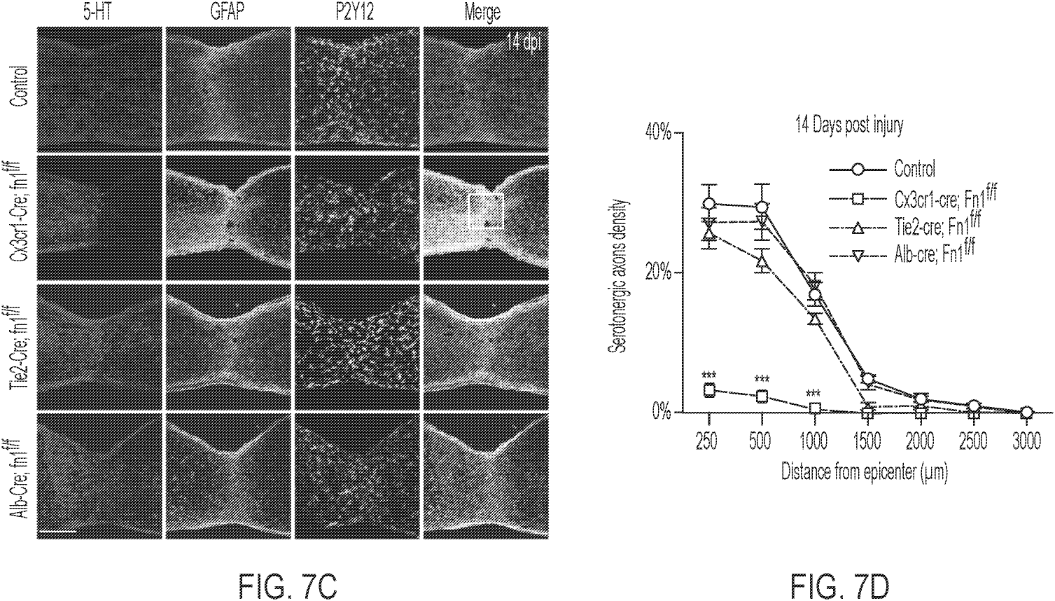

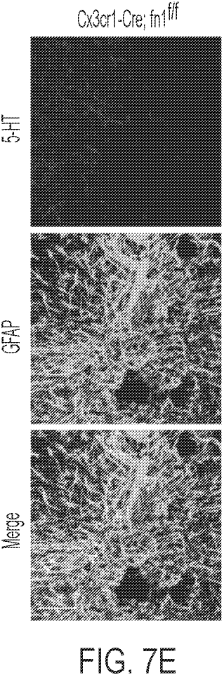

Functional experiments were performed based on the molecular signatures of MG3. First, the role of microglia-derived fibronectin was assessed as a component of the bridge formed between the two severed spinal ends (FIG. 3A) because of the observed transient expression of fibronectin and its binding proteins in MG3 cells (FIG. 5F). In addition to microglia, fibronectin at the lesion site could be from blood (synthesized in liver) and/or endothelial cell (Jaffe, E. A. & Mosher, D. F. Synthesis of fibronectin by cultured human endothelial cells. The Journal of experimental medicine 147, 1779-1791, doi:10.1084/jem.147.6.1779 (1978)). To selectively delete fibronectin from different cell types, Fn1^{flox/flox}mice were crossed with different Cre drivers (Cx3cr1-Cre for microglia (Yona, S. et al. Fate mapping reveals origins and dynamics of monocytes and tissue macrophages under homeostasis. Immunity 38, 79-91, doi:10.1016/j.immuni.2012.12.001 (2013)), Albumin-Cre for liver (Postic, C. et al. Dual roles for glucokinase in glucose homeostasis as determined by liver and pancreatic beta cell-specific gene knock-outs using Cre recombinase. The Journal of biological chemistry 274, 305-315, doi:10.1074/jbc.274.1.305 (1999)), and Tie2-Cre for endothelial cells (Kisanuki, Y. Y. et al. Tie2-Cre transgenic mice: a new model for endothelial cell lineage analysis in vivo. Developmental biology 230, 230-242, doi:10.1006/dbio.2000.0106 (2001))). P2 spinal crush injury was then performed. As shown in FIGS. 7A-7E, only Cx3cr1-Cre; Fn1^{flox/flox}mice exhibited a defect in the bridge formation at 3 dpi, with reduced axon growth at 14 dpi. These results indicate an important role of fibronectin secreted from microglia in mediating the initial steps in bridge formation for wound healing after spinal cord injury.

### Example 8: Proteinase Inhibitors Promote Healing

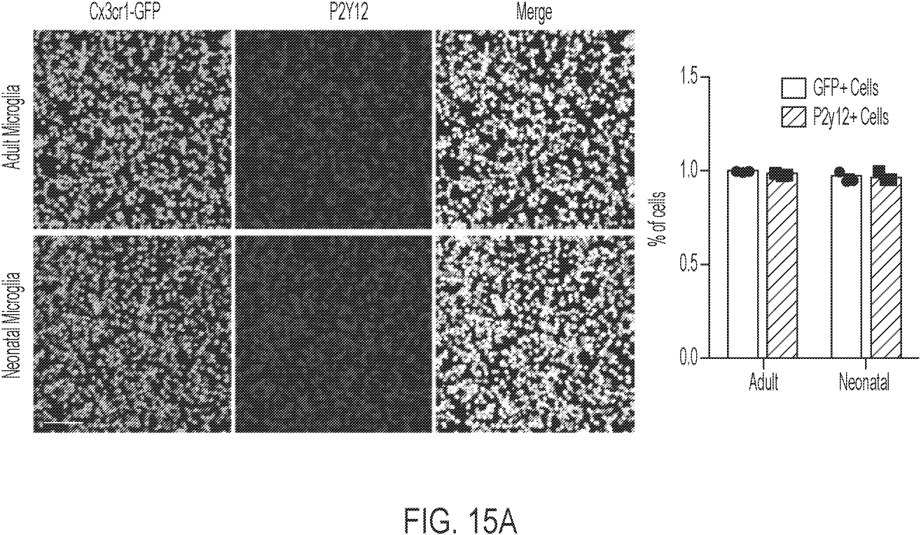

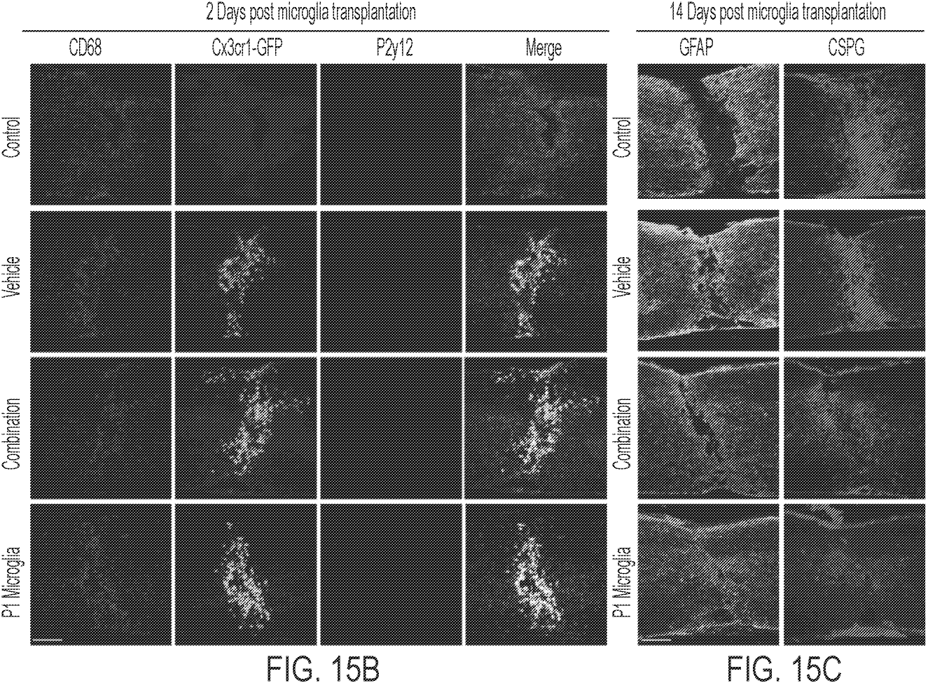

If the transiently expressed proteinase inhibitors identified through scRNA-Seq studies in neonatal spinal cord play a role in resolving inflammation, preventing scar formation, and thereby facilitating axon regrowth, then to test whether exogenously provided proteinase inhibitors altered injury responses in the adult spinal cord injury site, two chemical proteinase inhibitors were utilized: E64, a membrane-permeable irreversible inhibitor of a wide range of cysteine peptidases and serpina3n, a serine protease inhibitor (Hanada, K. et al. Isolation and Characterization of E-64, a New Thiol Protease Inhibitor. Agricultural and Biological Chemistry 42, 523-528, doi:10.1080/00021369.1978.10863014 (1978); Vicuna, L. et al. The serine protease inhibitor SerpinA3N attenuates neuropathic pain by inhibiting T cell-derived leukocyte elastase. Nature medicine 21, 518-523, doi:10.1038/nm.3852 (2015); Ou, J. et al. iPSCs from a Hibernator Provide a Platform for Studying Cold Adaptation and Its Potential Medical Applications. Cell 173, 851-863 e816, doi:10.1016/j.cell.2018.03.010 (2018)). To mimic the transient expression of proteinase inhibitors in MG3, microglia from adult Cx3cr1^{GFP/+}transgenic mice (FIG. 15A) were isolated, treated with either vehicle or E64/serpina3n combination, and then transplanted (with or without the inhibitors) into the adult spinal cord lesion site. As a control, microglia isolated from neonatal mice were also transplanted into the spinal cord lesion in a separate group of adult mice.

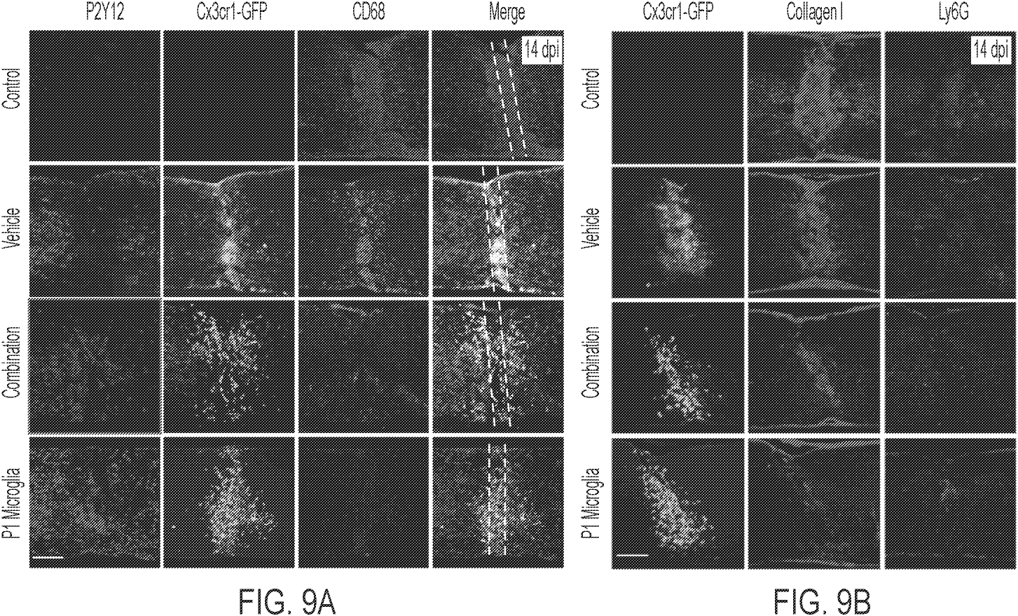

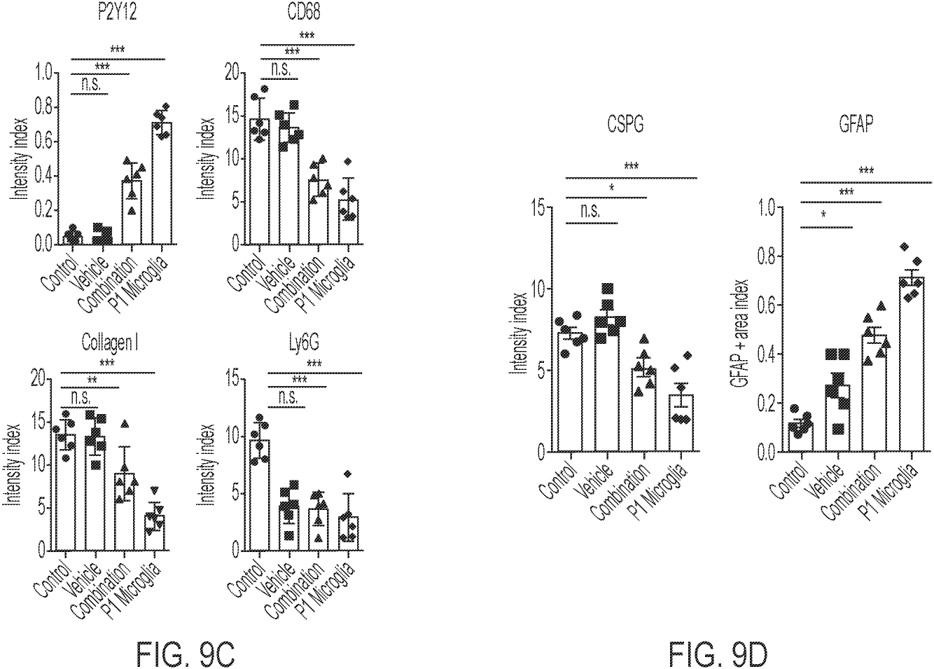

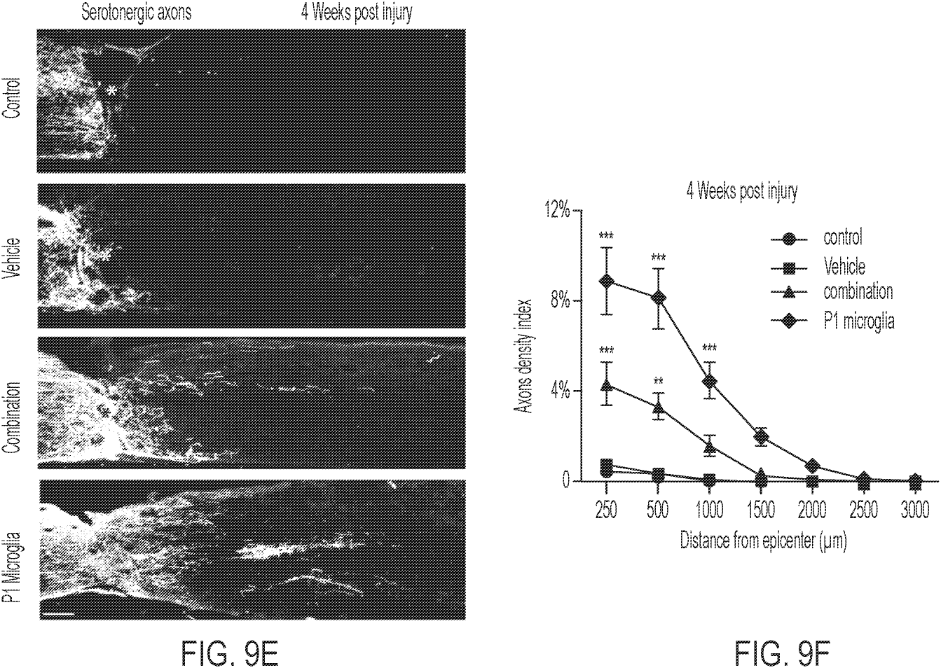

At 2 days after transplantation, most microglia were negatively stained with P2Y12 (FIG. 15B), suggesting their activation in the adult lesion. At 2 weeks after transplantation, both groups with adult microglia transplantation (with or without proteinase inhibitors treatments) exhibited significantly reduced infiltrated immune cells as indicated by Ly6G immunoreactivity and increased GFAP+ region (FIGS. 9B, 15C). Notably, transplanted adult microglia without proteinase inhibitors remained CD68+ and accumulated in the lesion (FIG. 9A), consistent with the corralling effects of adult microglia and macrophages in the lesion (Zhou, X. et al. Microglia and macrophages promote corralling, wound compaction and recovery after spinal cord injury via Plexin-B2. Nature neuroscience 23, 337-350, doi:10.1038/s41593-020-0597-7 (2020)). By contrast, many proteinase inhibitor-treated microglia expressed less CD68, re-expressed P2Y12, and some migrated into the host spinal tissues, similar to transplanted neonatal microglia (FIGS. 9A, 9C). In these mice, the lesion had significantly less deposition of collagen I and CSPG, but more serotonergic axons crossing the lesion (FIGS. 9B-9F, 15C). However, the number of regenerating axons in the mice with peptidase inhibitors-treated microglia was less than those observed with neonatal microglia transplantation (FIGS. 9E, 9F). Overall, proteinase inhibitors facilitate the reestablishment of homeostatic microglia and a permissive environment for axon regrowth after adult spinal cord injury.

Neonatal mice initiate scar-free wound healing with the capacity of spontaneous axon regrowth. These mice were able to achieve some degree of hindlimb locomotor function. Remarkably, microglia in neonatal mice were the primary coordinator of this reparative injury response, as deletion of these cells abolished neonatal scar-free healing, while transplantation of neonatal microglia into the adult lesions facilitated repair after spinal cord injury. In the acute phase of injury responses, these healing-promoting microglia played multiple roles, not only orchestrating efficient bridging between the cut ends, but also exhibiting remarkable inflammation resolution properties. These neonatal microglia experienced rapid state conversion; initially activated microglia rapidly reestablished homeostatic status. This feature of neonatal microglia differs from the permanent activation of microglia in the adult spinal cord lesions. Such temporal control of microglia activation is crucial for scar-free wound healing process. For example, transient expression of fibronectin and other related molecules are required for forming a bridge across the lesion site, but preventing scar formation. These results support a role of rapidly induced proteinase inhibitors in re-establishing homeostatic microglia in both neonatal and adult lesions.

These results were obtained using the following materials and methods.

### Animals

All experimental procedures were performed in compliance with animal protocols approved by the Institutional Animal Care and Use Committee at Boston Children’s Hospital. Wild-type C57BL/6 mice, Cx3cr1-Cre (JAX#025524), Tie2-Cre(JAX#008863), Alb-Cre(JAX#003574), Fn1^{flox} (JAX#029624), Csf1r^{flox}(JAX#021212), Ccr2^{RFP/RFP} (JAX#017586) and Cx3cr1^{GFP/GFP} mice (JAX#005582) were obtained from The Jackson Lab. Ccr2^{RFP/RFP} and Cx3cr1^{GFP/GFP} mice were bred to C57BL/6 background, and only heterozygous mice were used.

### Surgeries

### Neonatal Crush Injury

Newborn pups (P2/P7) were anesthetized by isoflurane. A laminectomy was performed at T10 until the spinal cord was exposed completely from side-to-side. The spinal cord was then fully crushed for 2 seconds with forceps with a width of 0.1 mm in the last 5 mm of tips. The muscles and skin were sutured in layers with 6-0 sutures. EMLA topical cream was used for post-surgery analgesia. Animals then were warmed up and kept in a box containing the bedding from the original cage for at least 30 minutes. After that, the closed surgical site was rubbed with the feces from the mom using a Q-tip and then returned to the mom. Feeding was closely monitored in the first week after surgery. Nutra-Gel diets (Bio-Serv) or breeder chow diets were provided to avoid cannibalism. Pups were sacrificed when they showed weight loss (more than 10%). In case of bladder dysfunction, bladder emptying was performed once a day until bladder function was restored.

### Adult Surgeries

Adult (and young adult) T10 crush injury was performed at thoracic level 10 (T10) (Liu, K. et al. PTEN deletion enhances the regenerative ability of adult corticospinal neurons. Nature neuroscience 13, 1075-1081, doi:10.1038/nn.2603 (2010)). Briefly, a midline incision was made over the thoracic vertebrae, followed by a T9-11 laminectomy. Tips of forceps were carefully inserted on either side of the cord to include the full width of the cord and then to gently scrape them across the bone on the ventral side to not spare any tissue ventrally or laterally. The spinal cord was fully crushed for 2 seconds with 0.1 mm forceps. The muscles were sutured, and the skin was closed with wound clips. Mice were placed on a warming pad after surgery until fully awake and given buprenorphine for pain relief (2 times per day for 3 days after SCI surgery, 2 times per day for 2 days after brain injection). Their bladders were manually expressed 2 times per day for the duration of the experiments. To label CST axons, AAV-ChR2-mCherry was injected to the mouse sensorimotor cortex (Chen, B. et al. Reactivation of Dormant Relay Pathways in Injured Spinal Cord by KCC2 Manipulations. Cell 174, 521-535 e513, doi:10.1016/j.cell.2018.06.005 (2018)). AAVs were generated at the viral core of the Boston Children’s Hospital and their titers were adjusted to 5×10¹² copies/ml for injection.

### Perfusion and Immunohistochemistry

Mice were given a lethal dose of anesthesia and transcardially perfused with PBS followed by 4% paraformaldehyde (PFA). PFA fixed tissues were incubated in 30% sucrose in PBS for 3 days at 4° C., embedded in OCT (Tissue-Tek), frozen in a dry ice/ethanol bath, and stored at -80° C. Transverse and sagittal sections were cut on a cryostat at 30 µm thick and stored at -20° C. until processed. Before staining, sections were warmed to room temperature and dried on a 37° C. slide warmer for 2 h. Sections then were treated with a blocking solution containing 10% normal donkey serum and 0.5% Triton-100 for 2 hours at room temperature. The primary antibodies used were: rabbit/goat anti-5-HT [Immunostar (20080/20079), 1: 5,000]; rabbit anti-GFAP [DAKO (Z0334), 1:600]; rat anti-GFAP [Thermo Fisher (13-0300), 1:600]; rabbit anti-P2Y12 [AnaSpec (AS-55043A), 1:200]; rat anti-CD68 [Bio-Rad (MCA1957), 1:600]; rabbit anti-Fibronectin [Millipore (AB2033), 1:200]; rabbit anti- Collagen I [Abcam (ab21286), 1:200]; chicken anti-GFP [Abcam (ab13970), 1:400]; goat anti-SPP1 [R&D (AF808), 1:400]; mouse anti-Ly6G/Ly6C [R&D (MAB1037), 1:200]; mouse anti-CSPG [Sigma-Aldrich (C8035), 1:200]; rabbit anti-CSPG [Sigma-Aldrich (L9393), 1:500]; rabbit anti-RFP [Abcam (ab34771), 1:400]. Secondary antibodies included: Alexa Fluor 488-conjugated donkey anti chicken/rabbit; Alexa Fluor 555-conjugated donkey anti goat/rabbit/mouse and Dylight 650-conjugated donkey anti rat (all from Invitrogen). Spinal cord transverse and sagittal sections were imaged with a confocal laser-scanning microscope (Zeiss 700 or Zeiss 710). Figures showing large longitudinal spinal cord section were acquired using Zeiss 710 with image stitching and/or stack software.

### Lesion Analysis and Quantification

The lesion site was defined and traced using Collagen I, Fibronectin, GFAP, P2Y12, CD68, CD31, CSPG, Laminin or Ly6G/Ly6C staining. Mice were perfused separately at 3, 7 or 14 days after spinal cord crush as described above to visualize lesion site. Cell nuclei were stained by incubation with DAPI for 30 seconds. Immunostaining intensity in the lesion site (100 µm width region in the epicenter) was measured using ImageJ software and normalized to the intact (proximal) region of the spinal cord.

### Axon Counting and Quantification

To quantify the regenerating/late-arriving 5-HT/CST axons after spinal cord crush, sagittal sections through the lesion were stained with antibodies against 5-HT or RFP. Images of 5-6 sections for each animal were taken under a 10X objective and used for quantification. A series of rectangular segments 100 µm wide and 400 µm high covering the dorsal-ventral aspect of the cord were superimposed onto the sagittal sections, starting from lesion center up to a defined distance caudal. After subtracting the background, the pixel value of each segment was normalized by dividing with the rostral segment (1 mm rostral). The results were presented as a ratio at different distances (axons density index).

### Microglia Depletion

To deplete microglia in vivo, adult mice were given the CSF1R inhibitor PLX3397 mixed into AIN-76A standard chow (Research Diets). The dose of PLX3397 was 290 mg/kg and respective controls received AIN-76A standard chow. For neonatal microglia depletion, pregnant mice were given the PLX3397 chow from E14 and newborn pups continued to receive subcutaneous injection of PLX3397 (50 mg/kg/day) on daily basis. To accomplish microglia depletion by genetic means, Cx3cr1-Cre mice were crossed with Csf1r^{flox/flox}mice to generate Cx3cr1-Cre; Csf1r^{flox/flox}mice.

### RNA Isolation, Bulk RNA-Seq Analysis

After a quick PBS perfusion, spinal cords of adult mice were rapidly dissected out before (intact), 3 days after or 5 days after the crush. The central 1 mm of the lower thoracic lesion including the lesion core and 0.5 mm rostral and caudal were then rapidly removed after PBS perfusion. Tissues were dissociated using Neural Tissue Dissociation Kit (P) (Miltenyi Biotec) and stained with CD45 and CD11b for 10 min at 4° C. Microglia were then FACS purified using the markers CD45 and CD11b. RNAs of CD45^{low} CD11b^{hi} cells were extracted using RNeasy Micro Kit following the manufacturer’s instructions. RNA quality was verified with an Agilent BioAnalyzer 2100. All experimental steps through RNA extraction were performed in triplicate for the control and experimental groups, with each replicate performed on a different day. The samples within a replicate were prepared on the same day, in a different order each replicate, to avoid any systemic errors from differences in timing.

RNA-sequencing was carried out by the UCLA Neuroscience Genomics Core. Briefly, RNA samples were sequenced by the UNGC. Samples were pooled and barcoded. Library was prepared using Nugen Ovation RNA Ultra Low Input (500 pg) +Kapa Hyper. Preparation included 75bp paird-end reads and sequencing run was carried out over 5 lanes. An average of 55 M reads were obtained. Reads were aligned to mouse GRCm38 reference genome using STAR (ver 2.4.0) and an average of 80.9±1.1(SE)% uniquely aligned rate were obtained. Read counts for refSeq genes (mm10) were generated by HTSeq 0.6.1. Low count genes were filtered and FPKM values were generated. Differentially expressed genes were identified using Limma package.

### Single Cell RNAseq Analysis

Single cell suspension from FACS-sorted microglia cells was prepared using 10x Genomics Chromium Single Cell 3′ Reagent Kits v3 (10X Genomics, Pleasanton, CA) according to manufacturer’s protocol. Quantity and quality of cDNA were assessed by Agilent 2100 expert High Sensitivity DNA Assay. cDNA samples were sequenced on 1 lane of NovaSeq 6000 S2 flowcell at UCLA Technology Center for Genomics and Bioinformatics. Reads were mapped to Mouse GrCm38 genome using Cell Ranger v.3.0.2. More than 380 million reads were obtained for each sample. Average number of genes detected was 3571 (SE±255). Confident read mapping rates were 91.7-93.4% with over 86.8% of reads in cells. Filtering genes and cells: Seurat package v3.1.1 was used to do analysis. For each condition (0 dpi, 3 dpi, 5 dpi), genes expressing in less than 5 cells were removed. Cells with mitochondria express level less than 5%, ribosome express level less than 30%, and number of features larger than 2000 were used.

### Removal of Doublets

Package DoubletFinder was used to remove doublet. SCTransform was applied in each condition. PCs 1 to 30 were used for pk identification. A doublet formation rate of 7.5% was considered as threshold. Cells identified as doublet were removed.

### Clustering & Cell Type Classification

After removing doublet, all Seurat objects were merged together. Cells with number of features larger than 500, number of RNA count smaller than 80000, mitochondria express percent less than 15% were used. LogNormalize method with scale factor of 10000 was used for normalization. FindVariableFeatures function was used to extract top 5000 variable features. Data was scaled according to mitochondria percent by ScaleData function. Clustering results were visualized using tSNE/UMAP plot. Function FindAllMarkers was used to find significant genes in each cluster. Cell types were identified by using several marker genes from literature.

### Subset and Cell Cycle Regression

Clusters identified as microglia-like cells were subsetted for further analysis. Cell cycle related genes were regressed out to remove cell cycle effects according to cell cycle phase scores, as instructed in Seurat vignette (satijalab.org/seurat/v3.1/cell_cycle_vignette_html). Same method was used to cluster and identify subtypes of microglia cells.

### GO Enrichment Analysis

Package DOSE 3.8.2 and database org.Mm.eg.db 3.7.0 were used for GO enrichment analysis. Genes with adjusted p-value less than 0.05 and log fold change larger than 0.25 were selected. Top 300 genes ranked by log fold change in cluster 3 (compare with cluster 0) were used as input. All three ontologies (BP, CC, MF) were exported.

### In Situ Hybridization in Sample on a Slide

To assess the expression pattern of Fn1, Ms4a7, P2ry12 and Thbs1, in situ hybridization was performed by hybridization chain reaction (HCR). Commercial in situ kit was purchased from Molecular Instruments (Molecularinstruments.org). Each kit containing a DNA probe set, a DNA HCR amplifier (comprising a pair of fluorophore-labeled DNA hairpins), and hybridization, wash and amplification buffers. The designed probes were synthesized by Integrated DNA Technologies. Each probe was designed for 20 or 40 probe sets, and the size of each probe set was based on the expression level of the target. Mice at multiple stages after crush were anesthetized with ketamine/xylazine and perfused with DEPC-PBS followed by 4% paraformaldehyde (PFA). Spinal cord was dissected and fixed in 4% PFA overnight. Dehydrated in 30% sucrose/DEPC-PBS at 4° C., embedded in OCT and cryosectioned at 30 µm, and slices were kept in -20° C. Tissues were permeabilized in 5% SDS for 30 min at room temperature (RT) and pre-hybridized in hybridization buffer for 3 hour at 37° C. Then the slides were incubated in pre-warmed hybridization buffer including probes (2.5 nM for each) at 37° C. overnight. After hybridization, slices were washed for 1 hour at 37° C. with wash buffer followed by 2xSSC for 15 min at RT. The amplification step was performed with HCR amplifiers (B1, B2, B3 or B4) for overnight at RT.

### Neonatal/Adult Microglia Isolation and Transplantation

Neonatal/adult microglia were isolated from the brain of CX3CR1^{GFP/+}mice. Mice were anesthetized with ketamine/xylazine (100/10 mg/kg) and perfused with cold PBS. Cerebral cortices from postnatal day 1 (P1) or 2 months old mice brain were dissected without Dura. Enzymatic digestion with Neural Tissue Dissociation Kit (P) (Miltenyi Biotec) was performed, dissociated cells were re-suspended in PBS buffer with 0.5% BSA and then were passed through a 70 µm cell strainer (Falcon). Myelin was removed using myelin removal beads II and the MACS system (Miltenyi Biotec). After myelin depletion, microglial cells were positively selected from cell suspensions using CD11b MicroBeads and MACS system (Miltenyi Biotec). Neonatal microglial were purified directly without the myelin removal step. For the combination treatment group, adult microglial cells (1^{∗}10⁶/ml) were incubated with 10 µM E64 and 100 ng/ml Serpina3n for 30 min, centrifuge at 300 g for 10 min and re-suspended to ~2^{∗}10⁵ /µl cells. 30 µM E64 and 500 ng/ml Serpina3n were added to the cells again during transplantation. Microglia transplantations were performed one hour after spinal cord injury. One microliter containing ~2^{∗}10⁵ /µl CD11b⁺ purified mouse microglia in PBS were slowly injected into the crushed sites by a Nanoliter injector (WPI) with a pulled glass microcapillary tube (WPI).

### Statistical Analysis

The normality of distributions and the equality of variances were tested by SAS before the application of any parametric analyses. Two-tailed Student’s t test was used for the pairwise comparison between two groups. The rest of the data were analyzed using one-way or two-way ANOVA depending on the appropriated design. Multiple comparison procedures were carried out to identify specific between-group differences using Bonferroni’s post hoc. Error bars in all figures represent mean ± SEM. Differences were considered statistically significant at a p value below 0.05. All data were analyzed using GraphPad Prism and SAS.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for reducing post-injury scar formation in the central nervous system of a subject, reducing neuroinflammation in a subject, or treating neurodegeneration in a subject the method comprising contacting a site of injury with a proteinase inhibitor and/or phospholipase A2 inhibitor and/or a microglial cell or microglial-like cell contacted with a proteinase inhibitor and/or phospholipase A2 inhibitor, thereby reducing post-injury scar formation.

2-3. (canceled)

4. The method of claim 1, wherein the proteinase inhibitor is a cysteine peptidase inhibitor or a serine protease inhibitor.

5. The method of claim 4, wherein the cysteine peptidase inhibitor is E64, the serine protease inhibitor is serpina3n, and/or the phospholipase A2 inhibitor is Varespladib.

6. The method of claim 1, wherein the microglial cell or microglial-like cell
i) expresses a SPP1 and/or a CD68 polypeptide or a polynucleotide encoding said polypeptide;
ii) fails to express or expresses reduced levels of a P2Y12 polypeptide or a polynucleotide encoding said polypeptide;
iii) has an ameboid morphology,
iv) expresses a polypeptide selected from the group consisting of Igf1, Ms4a7, Fabp5 Mif, Ms4a7, Thbs1, Clec7a, Ms4a7, Ms4a6c, Lgals1, fibronectin 1 (Fn1), thrombospondin 1 (Thbs1), a phospholipase A2 inhibitor, Cstb, Stfal and Serpinb6a, 6Anxal, or a polynucleotide encoding said polypeptide;
v) is derived from an induced pluripotent stem cell or embryonic stem cell; and/or
vi) is autologous or heterologous.

7-14. (canceled)

15. The method of claim 1, wherein the microglial cell or microglial-like cell is contacted with the proteinase inhibitor and/or phospholipase A2 inhibitor in vitro or in vivo.

16. The method of claim 1, wherein the site of injury, neuroinflammation, or neurodegeneration is the brain, optic nerve, or spinal cord or a traumatic injury or a post-surgical injury.

17. (canceled)

18. The method of claim 1, wherein the method promotes axon regeneration or regrowth.

19. The method of claim 1, wherein the proteinase inhibitor and/or phospholipase A2 inhibitor and the microglial cell or microglial-like cell treated with a proteinase inhibitor and/or phospholipase A2 inhibitor are administered concurrently or sequentially.

20-23. (canceled)

24. The method of claim 1, wherein the microglial cell or microglial-like cell is an activated microglial cell or microglial-like cell.

25. The method of claim 24, wherein the microglial cell or microglial-like cell expresses one or more markers associated with an MG2 or MG3 microglial cell.

26. The method of claim 1, wherein said administration or contacting reduces the number of CD68+ cells, fibroblasts, reactive astrocytes, collagen I, fibronectin, CSPG and/or laminin present at the site of injury, neuroinflammation, or neurodegeneration relative to an untreated site of injury, neuroinflammation, or neurodegeneration.

27. (canceled)

28. The method of claim 1, wherein the microglial cell or microglial-like cell is characterized as having the following polypeptide expression profile: CD68-, SPP1-, P2Y12+, TMEM119+.

29-30. (canceled)

31. The method of claim 3, wherein the neurodegeneration is associated with a disease selected from the group consisting of Alzheimer’s disease, Parkinson’s disease, glaucoma, metachromatic leuokodystrophy, adrenoleukodystophy, lysosomal storage disorders, multiple sclerosis and amyotrophic lateral sclerosis.

32. The method of claim 2, wherein the neuroinflammation is associated with a neuroinflammatory disease and/or neuronal injury.

33. The method of claim 32, wherein the neuronal injury is selected from the group consisting of traumatic brain injury, spinal cord injury, spinal cord crush, and optic nerve injury.

34. (canceled)

35. A pharmaceutical composition comprising an amount of a peptide inhibitor and/or phospholipase A2 inhibitor in an amount effective to reduce post-injury scar formation in the central nervous system of a subject, reduce inflammation, or treat neurodegeneration.

36. A pharmaceutical composition comprising a microglial cell or microglial like cell treated with one or more proteinase inhibitors and/or one or more phospholipase A2 inhibitors.

37. The pharmaceutical composition of claim 36, wherein the microglial cell or microglial like cell is characterized as having the following polypeptide expression profile: CD68-, SPP1-, P2Y12+ TMEM119+.

38-39. (canceled)

40. An isolated microglial-like cell treated with a proteinase inhibitor and/or phospholipase A2 inhibitor and characterized as having one of the following polypeptide expression profiles:
(i) CD68-, SPP1-, P2Y12+, TMEM119+; or
(ii) CD68-, SPP1-, P2Y12+, and/or TMEM119+.

41. A kit comprising the pharmaceutical composition of claim 35 .
